(19) United States
(12) Patent Application Publication
(10) Pub. No.: US 2024/0334932 A1
(43) Pub. Date: Oct. 10, 2024

(54) POLYMORPHS HAVING PESTICIDAL ACTIVITY

(71) Applicant: CORTEVA AGRISCIENCE LLC, Indianapolis, IN (US)

(72) Inventors: Rick BETORI, Indianapolis, IN (US); Negar GARIZI, Indianapolis, IN (US); Paul LARSEN, Indianapolis, IN (US); Jinglin LIU, Indianapolis, IN (US); Tony TRULLINGER, Indianapolis, IN (US); Nicola WEBB, Indianapolis, IN (US); Frank WESSELS, Indianapolis, IN (US); Neeraj SANE, Indianapolis, IN (US); Jennifer SKELTON, Indianapolis, IN (US)

(21) Appl. No.: 18/294,307

(22) PCT Filed: Jul. 29, 2022
(86) PCT No.: PCT/US2022/074322

§ 371 Date: Feb. 1, 2024

Related U.S. Application Data
(60) Provisional application No. 63/228,910, filed on Aug. 3, 2021.
(60) Provisional application No. 63/368,548, filed on Jul. 15, 2022.

(51) Int. Cl. A01N 43/78; A01P 7/04; C07D 417/04

(52) U.S. Cl. CPC A01N 43/78; A01P 7/04; C07D 417/04

(57) ABSTRACT

This disclosure relates to polymorphic forms of N-[4-chloro-2-(pyridin-3-yl)-1,3-thiazol-5-yl]-N -ethyl-3-(methylsulfonyl) propanamide, that are useful in the control of pests in the Order Hemiptera, Thysanoptera, Lepidoptera, and the like, processes to produce such polymorphic forms, intermediates used in such processes, pesticidal compositions containing such polymorphic forms, and processes of using such pesticidal compositions against such pests.

### CROSS-REFERENCE TO RELATED APPLICATIONS

This application claims priority under 35 U.S.C. § 119 (e) to U.S. Provisional Application Ser. No. 63/228,910 filed on Aug. 3, 2021 and U.S. Provisional Application Ser. No. 63/368,548 filed on Jul. 15, 2022, the entire disclosures of which are incorporated herein by reference.

### TECHNICAL FIELD

This disclosure relates to polymorphic forms of N-[4-chloro-2-(pyridin-3-yl)-1,3-thiazol-5-yl]-N-ethyl-3-(methylsulfonyl) propanamide, that are useful in the control of pests in the Order Hemiptera, Thysanoptera, Lepidoptera, and the like, processes to produce such polymorphic forms, intermediates used in such processes, pesticidal compositions containing such polymorphic forms, and processes of using such pesticidal compositions against such pests.

### BACKGROUND

“Many of the most dangerous human diseases are transmitted by insect vectors” (Rivero et al.). “Historically, malaria, dengue, yellow fever, plague, filariasis, louse-borne typhus, trypanosomiasis, leishmaniasis, and other vector-borne diseases were responsible for more human disease and death in the 17th through the early 20th centuries than all other causes combined” (Gubler). Vector-borne diseases are responsible for about 17% of the global parasitic and infectious diseases. Malaria alone causes over 800,000 deaths a year, 85% of which occur in children under five years of age. Each year there are about 50 to about 100 million cases of dengue fever. A further 250,000 to 500,000 cases of dengue hemorrhagic fever occur each year (Matthews). Vector control plays a critical role in the prevention and control of infectious diseases. However, insecticide resistance, including resistance to multiple insecticides, has arisen in all insect species that are major vectors of human diseases (Rivero et al.). Recently, more than 550 arthropod species have developed resistance to at least one pesticide (Whalon et al.). Furthermore, the cases of insect resistance continue to exceed by far the number of cases of herbicide and fungicide resistance (Sparks et al.).

Each year insects, plant pathogens, and weeds destroy more than 40% of all food production. This loss occurs despite the application of pesticides and the use of a wide array of non-chemical controls, such as crop rotations, and biological controls. If just some of this food could be saved, it could be used to feed the more than three billion people in the world who are malnourished (Pimental).

Plant parasitic nematodes are among the most widespread pests and are frequently one of the most insidious and costly. It has been estimated that losses attributable to nematodes are from about 9% in developed countries to about 15% in undeveloped countries. However, in the United States of America a survey of 35 States on various crops indicated nematode-derived losses of up to 25% (Nicol et al.).

It is noted that gastropods (slugs and snails) are pests of less economic importance than other arthropods or nematodes, but in certain places, they may reduce yields substantially, severely affecting the quality of harvested products, as well as, transmitting human, animal, and plant diseases. While only a few dozen species of gastropods are serious regional pests, a handful of species are important pests on a worldwide scale. In particular, gastropods affect a wide variety of agricultural and horticultural crops, such as, arable, pastoral, and fiber crops; vegetables; bush and tree fruits; herbs; and ornamentals (Speiser).

Termites cause damage to all kinds of private and public structures. The worldwide termite damage losses amount to billions of U.S. dollars each year. In 2005, it was estimated that termites cause over US$50 billion in damage worldwide each year (Korb).

Consequently, for many reasons, including those mentioned above, there is an on-going need for the costly (estimated to be about US$256 million per pesticide in 2010), time-consuming (on average about 10 years per pesticide), and difficult, development of new pesticides (CropLife America).

There is an acute need for new pesticides. Certain pests are developing resistance to pesticides in current use. Hundreds of pest species are resistant to one or more pesticides. The development of resistance to some of the older pesticides, such as DDT, the carbamates, and the organophosphates, is well known. But resistance has even developed to some of the newer pesticides.

Certain pesticides, such as insecticides, have been shown to eliminate insect pests by disrupting a physiological process that is essential to the development, reproduction, or survival of the target pest. These physiological disruptions can occur in a variety of ways, which has led to the discovery and development of compounds that act through many different modes of action. Pesticides falling into the category of insecticides, have various modes of action that can be broadly categorized into different groups based on which physiological process they disrupt. Exemplary modes of action include, nerve and muscle, growth and development, respiration, midgut targets, and insecticides with unknown or non-specific action (IRAC 2022). Although these are broad classifications, the cause of target pest mortality is not always congruent with the specific mode of action of an insecticide (Matsumura 1985).

One example is the of insecticides affecting chordotonal organs. Examples of such insecticides are commercially available, and include compounds such as pymetrozine, pyrifluquinazon and flonicamid, and newer members recently introduced to the market, such as afidopyropen. While broadly categorized as targeting nerve and muscle tissue, chordotonal modulators induce a variety of behavioral symptoms in target pest species, including the disruption of coordination and ability to feed, eventually leading to death due to starvation and desiccation (Kandasamy et al. 2017, Morita et al. 2007, Maienfisch 2019, Wang et al. 2011, Zhou et al. 2021). Behavioral studies have investigated the behavioral effect of chordotonal modulators. For example, Lee and colleagues (Lee et al. 2013) evaluated the effect of pyrifluquinazon on Bemisia tabaci and Trialeurodes vaporarium adults and noted a quick knock-down effect and strong symptoms of intoxication including convulsions and paralysis. A similar behavioral effect was reported in Drosophila melanogaster, where exposure to chordotonal modulators strongly inhibited climbing behavior in treated flies (Nesterov et al. 2015, Wang et al. 2019). Because chordotonal modulator insecticides affect mobility and feeding, the onset of mortality from starvation and desiccation occurs more slowly than other insecticides such as neonicotinoids (He et al. 2010, Maienfisch 2019). However, symptoms of intoxication are visible shortly after exposure and can be diagnostic of their action (Lee et al. 2013, Morita et al. 2007). As such, it has been shown that behavioral effect of chordotonal modulators, such as the knock-down effect, is a change that leads to increased mortality and can be provided as an indicator of mortality.

Therefore, for many reasons, including the above reasons, a need exists for new pesticides. One such compound, N-[4-chloro-2-(pyridin-3-yl)-1,3-thiazol-5-yl]-N-ethyl-3 -(methylsulfonyl) propanamide, or a solvate or hydrate thereof (also herein referred to as “Compound 1”, also known as N-(4-chloro-2-(pyridin-3-yl) thiazol-5-yl)-N-ethyl-3 -(methylsulfonyl) propanamide) represented by the formula 1

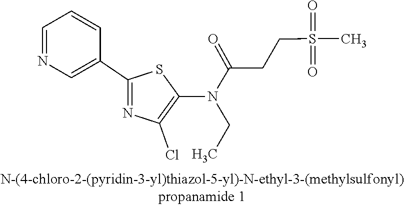 [SMILES: CCN(C(=O)CCS(C)(=O)=O)c1sc(-c2cccnc2)nc1Cl]

is a potent small-molecule showing activity against a variety of pests. Compound 1 is being investigated for insecticidal utility. Compounds related to Compound 1 are disclosed in International Patent Publication No. WO 2010/139497 A1 and U.S. Pat. No. 8,350,044, which are incorporated herein by reference in their entirety.

While Compound 1 has found application as a pesticide, it is advantageous to have polymorphic forms having improved properties, such as improved crystallinity, dissolution properties, decreased hygroscopicity, and/or ease of formulation in commercially viable compositions for application in the field, while maintaining chemical stability properties.

### CERTAIN REFERENCES CITED IN THE DISCLOSURE

Busvine, J. R. (1971). Contact poisons in solid form: residual films of contact insecticides. In A Critical Review of the Techniques for Testing Insecticides (Second Edition ed., pp. 102-128). Commonwealth Agricultural Bureaux.

CropLife America, The Cost of New Agrochemical Product Discovery, Development & Registration, and Research & Development predictions for the Future, 2010.

Drewes, M., Tietjen, K., Sparks, T. C., High-Throughput Screening in Agrochemical Research, Modern Methods in Crop Protection Research, Part I, Methods for the Design and Optimization of New Active Ingredients, Edited by Jeschke, P., Kramer, W., Schirmer, U., and Matthias W., p. 1-20, 2012.

Gubler, D., Resurgent Vector-Borne Diseases as a Global Health Problem, Emerging Infectious Diseases, Vol. 4, No. 3, p. 442-450, 1998.

He, Y., Chen, L., Chen, J., Zhang, J., Chen, L., Shen, J., & Cheng Zhu, Y. (2011). Electrical penetration graph evidence that pymetrozine toxicity to the rice brown planthopper is by inhibition of phloem feeding. Pest Management Science, 67 (4), 483-491.

IRAC Insecticide Resistance Action Committee (2022). IRAC Mode of Action Classification Scheme, Version 10.2. 39. Retrieved March 2022, from https://irac -online.org/mode-of-action/

Kandasamy, R., London, D., Stam, L., von Deyn, W., Zhao, X., Salgado, V. L., & Nesterov, A. (2017). Afidopyropen: New and potent modulator of insect transient receptor potential channels. Insect Biochemistry and Molecular Biology, 84, 32-39.

Korb, J., Termites, Current Biology, Vol. 17, No. 23, 2007.

Lee, S.-W., Song, M.-K., Ahn, Y. J., Kim, Y.-J., Moon, Y.-S., Koo, H. N., & Kim, G. H. (2013). Insecticidal Activity and Behavioral Disorders by Pyrifluquinazon to Trialeurodes vaporariorum and Bemisia tabaci. The Korean Journal of Pesticide Science, 17 (1), 33-40

Maienfisch, P. (2019). Selective feeding blockers: pymetrozine, flonicamid, and pyrifluquinazon. In P. Jeschke, M. Witschel, W. Krämer, & U. Schirmer (Eds.), Modern Crop Protection Compounds (Third Edition ed., Vol. Volume 3: Insecticides, pp. 1501-1526). Wiley-VCH.

Matsumura, F. (1985). Modes of action of insecticides. In Toxicology of Insecticides: Second Edition (pp. 111-202). Plenum Press.

Matthews, G., Integrated Vector Management: Controlling Vectors of Malaria and Other Insect Vector Borne Diseases, Ch. 1, p. 1, 2011.

Morita, M., Ueda, T., Yoneda, T., Koyanagi, T., & Haga, T. (2007). Flonicamid, a novel insecticide with a rapid inhibitory effect on aphid feeding. Pest Management Science, 63, 969-973.

Nesterov, A., Spalthoff, C., Kandasamy, R., Katana, R., Rankl, Nancy B., Andrés, M., Jähde, P., Dorsch, John A., Stam, Lynn F., Braun, F.-J., Warren, B., Salgado, Vincent L., & Göpfert, Martin C. (2015). TRP Channels in Insect Stretch Receptors as Insecticide Targets. Neuron, 86 (3), 665-671.

Nicol, J., Turner S., Coyne, L., den Nijs, L., Hocksland, L., Tahna-Maafi, Z., Current Nematode Threats to World Agriculture, Genomic and Molecular Genetics of Plant—Nematode Interactions, p. 21-43, 2011.

Pimental, D., Pest Control in World Agriculture, Agricultural Sciences—Vol., II, 2009.

Rivero, A., Vezilier, J., Weill, M., Read, A., Gandon, S., Insect Control of Vector-Borne Diseases: When Is Insect Resistance a Problem? Public Library of Science Pathogens, Vol. 6, No. 8, p. 1-9, 2010.

Sparks T. C., Nauen R., IRAC: Mode of action classification and insecticide resistance management, Pesticide Biochemistry and Physiology (2014) available online 4 Dec. 2014.

Speiser, B., Molluscicides, Encyclopedia of Pest Management, Ch. 219, p. 506-508, 2002.

Stroup, W. W. (2012). Generalized Linear Mixed Models: Modern Concepts, Methods and Applications. CRC Press. 555 pp.

Wang, H., Lei, Z., Mei, Y., Shuo, L., & YunLiang, J. (2011). Effect of pymetrozine interferes with feeding behavior of Bemisia tabaci (Homoptera, Aleyrodidae). Chinese Journal of Applied Entomology, 48 (1), 54-59.

Wang, L.-X., Niu, C.-D., Salgado, V. L., Lelito, K., Stam, L., Jia, Y.-L., Zhang, Y., Gao, C.-F., & Wu, S.-F. (2019). Pymetrozine activates TRPV channels of brown planthopper Nilaparvata lugens. Pesticide Biochemistry and Physiology, 153, 77-86.

Whalon, M., Mota-Sanchez, D., Hollingworth, R., Analysis of Global Pesticide Resistance in Arthropods, Global Pesticide Resistance in Arthropods, Ch. 1, p. 5-33, 2008.

Zhou, X., Zhang, Z., Zheng, H., Zhang, Q., Gong, J., Li, C., & Wang, R. (2021). Physiological and Biochemical Responses to Sublethal Concentrations of the Novel Pyropene Insecticide, Afidopyropen, in Whitefly Bemisia tabaci MED (Q Biotype). Agronomy, 11 (11).

### DEFINITIONS OF THE DISCLOSURE

The examples given in these definitions are generally non-exhaustive and must not be construed as limiting this disclosure. It is understood that a substituent should comply with chemical bonding rules and steric compatibility constraints in relation to the particular molecule to which it is attached. These definitions are only to be used for the purposes of this disclosure.

The phrase “active ingredient” means a material having activity useful in controlling pests, and/or that is useful in helping other materials have better activity in controlling pests; examples of such materials include, but are not limited to, acaricides, algicides, antifeedants, avicides, bactericides, bird repellents, chemosterilants, fungicides, herbicide safeners, herbicides, insect attractants, insect repellents, insecticides, mammal repellents, mating disrupters, molluscicides, nematicides, plant activators, plant growth regulators, rodenticides, synergists, and virucides (see bcpc.org).

The phrase “active ingredient group alpha” (hereafter “AIGA”) means collectively the following materials: abamectin, abamectin-aminomethyl, abscisic acid, ACC, acephate, acequinocyl, acetamiprid, acethion, acetochlor, acetofenate, acetophos, acetoprole, acibenzolar, acifluorfen, aclonifen, ACN, acrep, acrinathrin, acrolein, acrylonitrile, acynonapyr, acypetacs, afidopyropen, afoxolaner, (S)-afoxolaner, AITC, alachlor, alanap, alanycarb, albendazole, aldicarb, aldicarb sulfone, aldimorph, aldoxycarb, aldrin, allethrin, d-trans-allethrin, allicin, allidochlor, allosamidin, alloxydim, allyl alcohol, allyl isothiocyanate, allyxycarb, alorac, alpha-bromadiolone, alpha-cypermethrin, alpha-endosulfan, alphamethrin, altretamine, aluminium phosphide, aluminum phosphide, ametoctradin, ametridione, ametryn, ametryne, amibuzin, amicarbazone, amicarthiazol, amidithion, amidochlor, amidoflumet, amidosulfuron, aminocarb, aminocyclopyrachlor, aminopyralid, 4-aminopyridine, aminopyrifen, aminotriazole, amiprofos-methyl, amiprophos, amiprophos-methyl, amisulbrom, amiton, amitraz, amitrole, ammonium sulfamate, amobam, amorphous silica gel, amorphous silicon dioxide, ampropylfos, AMS, anabasine, ancymidol, anilazine, anilofos, anisiflupurin, anisuron, anthraquinone, antimony potassium tartrate, antu, apholate, aramite, arprocarb, arsenous oxide, asomate, asulam, athidathion, atraton, atrazine, aureofungin, avermectin B₁, AVG, aviglycine, azaconazole, azadirachtin, azafenidin, azamethiphos, azidithion, azimsulfuron, azinphos-ethyl, azinphosethyl, azinphos-methyl, azinphosmethyl, aziprotryn, aziprotryne, azithiram, azobenzene, azocyclotin, azothoate, azoxystrobin, bachmedesh, barban, barbanate, barium hexafluorosilicate, barium polysulfide, barium silicofluoride, barthrin, basic copper carbonate, basic copper chloride, basic copper sulfate, BCPC, beflubutamid, beflubutamid-M, benalaxyl, benalaxyl-M, benazolin, bencarbazone, benclothiaz, bendaqingbingzhi, bendiocarb, bendioxide, benefin, benfluralin, benfuracarb, benfuresate, benmihuangcaoan, benodanil, benomyl, benoxacor, benoxafos, benquinox, benquitrione, bensulfuron, bensulide, bensultap, bentaluron, bentazon, bentazone, benthiavalicarb, benthiazole, benthiocarb, bentranil, benzadox, benzalkonium chloride, benzamacril, benzamizole, benzamorf, benzene hexachloride, benzfendizone, benzimine, benzipram, benzobicyclon, benzoepin, benzofenap, benzofluor, benzohydroxamic acid, benzomate, benzophosphate, benzothiadiazole, benzovindiflupyr, benzoximate, benzoylprop, benzpyrimoxan, benzthiazuron, benzuocaotong, benzyladenine, benzyl benzoate, berberine, beta-cyfluthrin, beta-cypermethrin, bethoxazin, BHC, gamma-BHC, bialaphos, bicyclopyrone, bifenazate, bifenox, bifenthrin, kappa-bifenthrin, bifujunzhi, bilanafos, binapacryl, binghuánzuò, bingqingxiao, bioallethrin, S-bioallethrin, bioethanomethrin, biopermethrin, bioresmethrin, biphenyl, bipyrazone, bisazir, bismerthiazol, bismerthiazol-copper, bisphenylmercury methylenedi (x-naphthalene-y-sulphonate), bispyribac, bistrifluron, bisultap, bitertanol, bithionol, bixafen, bixlozone, blasticidin-S, borax, Bordeaux mixture, boric acid, boscalid, BPCMS, BPMC, BPPS, brassinolide, brassinolide-ethyl, brevicomin, brodifacoum, brofenprox, brofenvalerate, broflanilide, brofluthrinate, bromacil, bromadiolone, alpha-bromadiolone, bromchlophos, bromethalin, bromethrin, bromfenvinfos, bromoacetamide, bromobonil, bromobutide, bromociclen, bromocyclen, bromo-DDT, bromofenoxim, bromofos, bromomethane, bromophos, bromophos-ethyl, bromopropylate, bromothalonil, bromoxynil, brompyrazon, brompyrazone, bromuconazole, bronopol, bropropdifacoum, BRP, BTH, bucarpolate, bufencarb, buminafos, bupirimate, buprofezin, Burgundy mixture, busulfan, busulphan, butacarb, butachlor, butafenacil, butam, butamifos, butane-fipronil, butathiofos, butenachlor, butene-fipronil, butethrin, buthidazole, buthiobate, buthiuron, butifos, butocarboxim, butonate, butopyronoxyl, butoxycarboxim, butralin, butrizol, butroxydim, buturon, butylamine, butylate, butylchlorophos, butylene-fipronil, cacodylic acid, cadusafos, cafenstrole, calciferol, calcium arsenate, calcium chlorate, calcium cyanamide, calcium cyanide, calcium polysulfide, calvinphos, cambendichlor, camphechlor, camphor, d-camphor, cantrodifene, captafol, captan, carbam, carbamorph, carbanolate, carbaril, carbaryl, carbasulam, carbathiin, carbathion, carbendazim, carbendazol, carbetamide, carbofenotion, carbofuran, carbon disulfide, carbon tetrachloride, carbonyl sulfide, carbophenothion, carbophos, carbosulfan, carboxazole, carboxide, carboxin, carfentrazone, carpropamid, cartap, carvacrol, carvone, CAVP, CDAA, CDEA, CDEC, cellocidin, CEPC, ceralure, cerenox, cetoctaelat, cevadilla, Cheshunt mixture, chinalphos, chinalphos-méthyl, chinomethionat, chinomethionate, chiralaxyl, chitosan, chlobenthiazone, chlomethoxyfen, chlor-IPC, chloralose, chloramben, chloramine phosphorus, chloramizol, chloramphenicol, chloraniformethan, chloranil, chloranocryl, chlorantraniliprole, chlorazifop, chlorazine, chlorbenside, chlorbenzuron, chlorbicyclen, chlorbromuron, chlorbufam, chlordane, chlordecone, chlordimeform, chlorempenthrin, chloretazate, chlorethephon, chlorethoxyfos, chloreturon, chlorfenac, chlorfenapyr, chlorfenazole, chlorfenethol, chlorfenidim, chlorfenprop, chlorfenson, chlorfensulphide, chlorfenvinphos, chlorfenvinphos-methyl, chlorfluazuron, chlorflurazole, chlorflurecol, chlorfluren, chlorflurenol, chloridazon, chlorimuron, chlorinate, chlormephos, chlormequat, chlormesulone, chlormethoxynil, chlornidine, chlornitrofen, chloroacetic acid, chlorobenzilate, chlorodinitronaphthalenes, chlorofénizon, chloroform, a-chlorohydrin, chloroinconazide, chloromebuform, chloromethiuron, chloroneb, chlorophacinone, chlorophos, chlorophthalim, chloropicrin, chloropon, chloroprallethrin, chloropropylate, chlorothalonil, chlorotoluron, chloroxifenidim, chloroxuron, chloroxynil, chlorphonium, chlorphoxim, chlorphthalim, chlorprazophos, chlorprocarb, chlorpropham, chlorpyrifos, chlorpyrifos-methyl, chlorquinox, chlorsulfuron, chlorthal, chlorthiamid, chlorthiophos, chlortoluron, chlozolinate, chltosan, cholecalciferol, choline chloride, chromafenozide, cicloheximide, cimectacarb, cimetacarb, cinerin I, cinerin II, cinerins, cinidon-ethyl, cinmethylin, cinosulfuron, cintofen, ciobutide, cisanilide, cismethrin, clacyfos, clefoxydim, clenpirin, clenpyrin, clethodim, climbazole, cliodinate, clodinafop, cloethocarb, clofencet, clofenotane, clofentezine, clofenvinfos, clofibric acid, clofop, clomazone, clomeprop, clonitralid, clopindol, cloprop, cloproxydim, clopyralid, cloquintocet, cloransulam, closantel, clothianidin, clotrimazole, cloxyfonac, cloxylacon, clozylacon, CMA, CMMP, CMP, CMU, codlelure, colecalciferol, colophonate, copper acetate, copper acetoarsenite, copper arsenate, copper carbonate, basic, copper hydroxide, copper naphthenate, copper oleate, copper oxychloride, copper 8-quinolinolate, copper silicate, copper sulfate, copper sulfate, basic, copper zinc chromate, coumachlor, coumafène, coumafos, coumafuryl, coumaphos, coumatetralyl, coumethoxystrobin, coumithoate, coumoxystrobin, 4-CPA, 4-CPB, CPMC, CPMF, 4-CPP, CPPC, credazine, cresol, cresylic acid, crimidine, croconazole, crotamiton, crotoxyfos, crotoxyphos, crufomate, cryolite, cue-lure, cufraneb, cumyleron, cumyluron, cuprobam, cuprous oxide, curcumenol, CVMP, cyanamide, cyanatryn, cyanazine, cyanofenphos, cyanogen, cyanophos, cyanthoate, cyantraniliprole, cyanuric acid, cyazofamid, cybutryne, cyclafuramid, cyclanilide, cyclaniliprole, cyclethrin, cycloate, cyclobutrifluram, cycloheximide, cycloprate, cycloprothrin, cyclopyranil, cyclopyrimorate, cyclosulfamuron, cycloxaprid, cycloxydim, cycluron, cyenopyrafen, cyetpyrafen, cyflufenamid, cyflumetofen, cyfluthrin, beta-cyfluthrin, cyhalodiamide, cyhalofop, cyhalothrin, gamma-cyhalothrin, lambda-cyhalothrin, cyhexatin, cymergan, cymiazole, cymoxanil, cyometrinil, cypendazole, cypermethrin, alpha-cypermethrin, beta-cypermethrin, theta-cypermethrin, zeta-cypermethrin, cyperquat, cyphenothrin, cyprazine, cyprazole, cyproconazole, cyprodinil, cyproflanilide, cyprofuram, cypromid, cyprosulfamide, cypyrafluone, cyromazine, cythioate, cytrex, 1,3-D, 2,4-D, 3,4-DA, daimuron, dalapon, daminozide, dayoutong, dazomet, 2,4-DB, 3,4-DB, DBCP, d-camphor, DCB, DCD, DCIP, DCPA (USA), DCPA (Japan), DCPTA, DCU, DDD, DDPP, DDT, pp′-DDT, DDVP, 2,4-DEB, debacarb, decafentin, decamethrin, decarbofuran, deet, dehydroacetic acid, deiquat, delachlor, delnav, deltamethrin, demephion, demephion-O, demephion-S, demeton, demeton-methyl, demeton-O, demeton-O-methyl, demeton-S, demeton-S-methyl, demeton-S-methylsulphon, demeton-S-methyl sulphone, DEP, 2,4-DEP, depalléthrine, derris, 2,4-DES, desmedipham, desmetryn, desmetryne, d-fanshiluquebingjuzhi, DFDT, diafenthiuron, dialifor, dialifos, di-allate, diallate, diamidafos, diamiphenethide, dianat, diatomaceous earth, diatomite, diazinon, dibrom, 1,2-dibromoethane, dibutyl phthalate, dibutyl succinate, dicamba, dicapthon, dicarbasulf, dicarbosulf, dichlobenil, dichlobentiazox, dichlobenz-methyl, dichlofenthion, dichlofluanid, dichlone, dichloralurea, dichlorbenzuron, dichlorfenidim, dichlorflurecol, dichlorflurenol, dichlormate, dichlormid, o-dichlorobenzene, ortho-dichlorobenzene, p-dichlorobenzene, para-dichlorobenzene, 2,5-dichlorobenzoic acid, 1,2-dichloroethane, dichloromethane, dichlorophen, 3,6-dichloropicolinic acid, 1,2-dichloropropane, 1,3-dichloropropene, dichlorprop, dichlorprop-P, dichlorvos, dichlozolin, dichlozoline, diclobutrazol, diclocymet, diclofop, diclomezine, dicloran, dicloromezotiaz, diclosulam, dicofol, dicophane, dicoumarol, dicresyl, dicrotophos, dicryl, dicumarol, dicyclanil, dicyclonon, dieldrin, dienochlor, diethamquat, diethatyl, diethion, diéthion, diethofencarb, dietholate, diéthon, diethyl pyrocarbonate, diethyltoluamide, difenacoum, difenoconazole, difenopenten, difenoxuron, difenzoquat, difethialone, diflovidazin, diflubenzuron, diflufenican, diflufenicanil, diflufenzopyr, diflumetorim, dikegulac, dilor, dimatif, dimefluazole, dimefluthrin, dimefox, dimefuron, dimehypo, dimenoxypyrin, dimepiperate, dimesulfazet, dimetachlone, dimetan, dimethacarb, dimethachlone, dimethachlor, dimethametryn, dimethenamid, dimethenamid-P, dimethipin, dimethirimol, dimethoate, dimethomorph, dimethrin, dimethyl carbate, dimethyl disulfide, dimethyl phthalate, dimethylvinphos, dimetilan, dimexano, dimidazon, dimoxystrobin, dimpropyridaz, dimpylate, dimuron, dinex, dingjunezuo, diniconazole, diniconazole-M, R-diniconazole, dinitramine, dinitrophenols, dinobuton, dinocap, dinocap-4, dinocap-6, dinocton, dinofenate, dinopenton, dinoprop, dinosam, dinoseb, dinosulfon, dinotefuran, dinoterb, dinoterbon, diofenolan, dioxabenzofos, dioxacarb, dioxathion, dioxation, dioxopyritrione, diphacin, diphacinone, diphenadione, diphenamid, diphenamide, diphenylamine, diphenyl sulfone, diphenyl sulphide, diprogulic acid, dipropalin, dipropetryn, dipterex, dipymetitrone, dipyrithione, diquat, disosultap, disparlure, disugran, disul, disulfiram, disulfoton, ditalimfos, dithianon, dithicrofos, dithioether, dithiométon, dithiopyr, dithiuron, diuron, dixanthogen, d-limonene, DMDS, DMPA, DNOC, dodemorph, dodicin, dodine, dofenapyn, doguadine, dominicalure, doramectin, 2,4-DP, 3,4-DP, DPC, drazoxolon, DSMA, d-teflumethrin, d-trans-allethrin, d-trans-resmethrin, dufulin, dymron, EBEP, EBP, ebufos, α-ecdysone, β-ecdysone, ecdysterone, echlomezol, EDB, EDC, EDDP, edifenphos, eglinazine, emamectin, EMPC, empenthrin, enadenine, endosulfan, alpha-endosulfan, endothal, endothall, endothion, endrin, enestroburin, enilconazole, enoxastrobin, ephirsulfonate, 24-epibrassinolide, EPN, epocholeone, epofenonane, epoxiconazole, eprinomectin, epronaz, epsilon-metofluthrin, epsilon-momfluorothrin, EPTC, epyrifenacil, erbon, ergocalciferol, erlujixiancaoan, esafoxolaner, esdépalléthrine, esfenvalerate, ESP, esprocarb, etacelasil, etaconazole, etaphos, etem, ethaboxam, ethachlor, ethalfluralin, ethametsulfuron, ethaprochlor, ethephon, ethidimuron, ethiofencarb, ethiolate, ethion, ethiozin, ethiprole, ethirimol, ethoate-methyl, ethobenzanid, ethofumesate, ethohexadiol, ethoprop, ethoprophos, ethoxyfen, (3-ethoxypropyl) mercury bromide, ethoxyquin, ethoxysulfuron, ethychlozate, ethylan, ethyl-DDD, ethylene, ethylene dibromide, ethylene dichloride, ethylene oxide, ethyl formate, ethylicin, ethylmercury acetate, ethylmercury bromide, ethylmercury chloride, ethylmercury 2,3-dihydroxypropyl mercaptide, ethylmercury phosphate, N-(ethylmercury)-p-toluenesulfonanilide, N-(ethylmercury)-p-toluenesulphonanilide, ethyl pyrophosphate, ethylthiometon, ethyltrianol, etiltrianol, etinofen, ETM, etnipromid, etobenzanid, etofenprox, etoxazole, etridiazole, etrimfos, eugenol, EXD, famoxacarb, famoxadone, famphur, d-fanshiluquebingjuzhi, fenac, fenamidone, fenaminosulf, fenaminstrobin, fenamiphos, fenapanil, fenarimol, fenasulam, fenazaflor, fenazaquin, fenbuconazole, fenbutatin oxide, fenchlorazole, fenchlorphos, fenclofos, fenclorim, fenethacarb, fenetrazole, fenfluthrin, fenfuram, fenhexamid, fenidim, fenitropan, fenitrothion, fénizon, fenjuntong, fenmezoditiaz, fenobucarb, fenolovo, fenoprop, fenothiocarb, fenoxacrim, fenoxanil, fenoxaprop, fenoxaprop-P, fenoxasulfone, fenoxycarb, fenpiclonil, fenpicoxamid, fenpirithrin, fenpropathrin, fenpropidin, fenpropimorph, fenpyrazamine, fenpyrazone, fenpyroximate, fenquinotrione, fenridazon, fenson, fensulfothion, fenteracol, fenthiaprop, fenthion, fenthion-ethyl, fentiaprop, fentin, fentrazamide, fentrifanil, fenuron, fenuron-TCA, fenvalerate, ferbam, ferimzone, ferric phosphate, ferrous sulfate, fipronil, flamprop, flamprop-M, flazasulfuron, flocoumafen, flometoquin, flonicamid, florasulam, florpyrauxifen, florylpicoxamid, fluacrypyrim, fluazaindolizine, fluazifop, fluazifop-P, fluazinam, fluazolate, fluazuron, flubendiamide, flubeneteram, flubenzimine, flubrocythrinate, flucarbazone, flucetosulfuron, fluchloralin, fluchloraminopyr, fluchlordiniliprole, flucofuron, flucycloxuron, flucythrinate, fludioxonil, fluénéthyl, fluenetil, fluensulfone, flufenacet, flufenerim, flufenican, flufenoxadiazam, flufenoxuron, flufenoxystrobin, flufenprox, flufenpyr, flufenzine, flufiprole, fluhexafon, fluindapyr, flumethrin, flumetover, flumetralin, flumetsulam, flumetylsulforim, flumezin, flumiclorac, flumioxazin, flumipropyn, flumorph, fluometuron, fluopicolide, fluopimomide, fluopyram, fluorbenside, fluoridamid, fluoroacetamide, fluoroacetic acid, fluorochloridone, fluoro-DDT, fluorodifen, fluorogesarol, fluoroglycofen, fluoroimide, fluoromide, fluoromidine, fluoronitrofen, fluoroxypyr, fluothiuron, fluotrimazole, fluoxapiprolin, fluoxastrobin, fluoxytioconazole, flupentiofenox, flupoxam, flupropacil, flupropadine, flupropanate, flupyradifurone, flupyrazofos, flupyrimin, flupyrsulfuron, fluquinconazole, fluralaner, flurazole, flurecol, flurenol, fluridone, flurochloridone, fluromidine, fluroxypyr, flurprimidol, flursulamid, flurtamone, flusilazole, flusulfamide, flutenzine, fluthiacet, fluthiamide, flutianil, flutolanil, flutriafol, fluvalinate, tau-fluvalinate, fluxametamide, fluxapyroxad, fluxofenim, folpel, folpet, fomesafen, fonofos, foramsulfuron, forchlorfenuron, formaldehyde, formetanate, formothion, formparanate, fosamine, fosetyl, fosmethilan, fospirate, fosthiazate, fosthietan, frontalin, fthalide, fuberidazole, fucaojing, fucaomi, fufenozide, fujunmanzhi, fulumi, fumarin, funaihecaoling, fuphenthiourea, furalane, furalaxyl, furamethrin, furametpyr, furan tebufenozide, furathiocarb, furcarbanil, furconazole, furconazole-cis, furethrin, furfural, furilazole, furmecyclox, furophanate, furyloxyfen, gamma-BHC, gamma-cyhalothrin, gamma-HCH, genit, gibberellic acid, gibberellin A3, gibberellins, gliftor, glitor, glucochloralose, glufosinate, glufosinate-P, L-glufosinate, glyodin, glyoxime, glyphosate, glyphosine, gossyplure, grandlure, griseofulvin, guanoctine, guazatine, halacrinate, halauxifen, halfenprox, halofenozide, halosafen, halosulfuron, haloxydine, haloxyfop, haloxyfop-P, haloxyfop-R, HCA, HCB, HCH, gamma-HCH, hemel, hempa, HEOD, heptachlor, heptafluthrin, heptamaloxyloglucan, heptenophos, heptopargil, herbimycin, herbimycin A, heterophos, hexachlor, hexachloran, hexachloroacetone, hexachlorobenzene, hexachlorobutadiene, hexachlorophene, hexaconazole, hexaflumuron, hexafluoramin, hexaflurate, hexalure, hexamide, hexazinone, hexylthiofos, hexythiazox, HHDN, holosulf, homobrassinolide, huanbifucaotong, huancaiwo, huanchongjing, huangcaoling, huanjunzuo, hydramethylnon, hydrargaphen, hydrated lime, hydrogen cyanamide, hydrogen cyanide, hydroprene, S-hydroprene, hydroxyisoxazole, 4-hydroxyphenethyl alcohol, 8-hydroxyquinoline sulfate, hymexazol, hyquincarb, IAA, IBA, IBP, icaridin, imazalil, imazamethabenz, imazamethapyr, imazamox, imazapic, imazapyr, imazaquin, imazethapyr, imazosulfuron, imibenconazole, imicyafos, imidacloprid, imidaclothiz, iminoctadine, imiprothrin, inabenfide, indanofan, indazapyroxamet, indaziflam, indoxacarb, inezin, infusorial earth, inpyrfluxam, iodobonil, iodocarb, iodofenphos, iodomethane, iodosulfuron, iofensulfuron, ioxynil, ipazine, IPBC, IPC, ipconazole, ipfencarbazone, ipfentrifluconazole, ipflufenoquin, iprobenfos, iprodione, iprovalicarb, iprymidam, ipsdienol, ipsenol, IPSP, IPX, isamidofos, isazofos, isobenzan, isocarbamid, isocarbamide, isocarbophos, isocil, isocycloseram, isodrin, isofenphos, isofenphos-methyl, isofetamid, isoflucypram, isolan, isomethiozin, isonoruron, isopamphos, isopolinate, isoprocarb, isoprocil, isopropalin, isopropazol, isoprothiolane, isoproturon, isopyrazam, isopyrimol, isothioate, isotianil, isouron, isovaledione, isoxaben, isoxacarbole, isoxachlortole, isoxadifen, isoxaflutole, isoxapyrifop, isoxathion, isuron, ivermectin, ixoxaben, izopamfos, izopamphos, japonilure, japothrins, jasmolin I, jasmolin II, jasmonic acid, jiahuangchongzong, jiajizengxiaolin, jiaxiangjunzhi, jiecaowan, jiecaoxi, Jinganmycin A, jodfenphos, juvenile hormone I, juvenile hormone II, juvenile hormone III, kadethrin, kappa-bifenthrin, kappa-tefluthrin, karbutilate, karetazan, kasugamycin, kejunlin, kelevan, ketospiradox, kieselguhr, kinetin, kinoprene, S-kinoprene, kiralaxyl, kresoxim-methyl, kuicaoxi, lactofen, lambda-cyhalothrin, lancotrione, latilure, lead arsenate, lenacil, lepimectin, leptophos, L-glufosinate, lianbenjingzhi, lime sulfur, d-limonene, lindane, lineatin, linuron, lirimfos, litlure, looplure, lotilaner, lufenuron, lüfuqingchongxianan, lüxiancaolin, lvdingjunzhi, lvfumijvzhi, lvxiancaolin, lythidathion, M-74, M-81, MAA, magnesium phosphide, malathion, maldison, maleic hydrazide, malonoben, maltodextrin, MAMA, manam, mancopper, mancozeb, mandestrobin, mandipropamid, maneb, matrine, mazidox, MCA, MCC, MCP, 1-MCP, MCPA, 2,4-MCPA, MCPA-thioethyl, MCPB, 2,4-MCPB, MCPP, mebenil, mecarbam, mecarbinzid, mecarphon, mecoprop, mecoprop-P, medimeform, medinoterb, medlure, mefenacet, mefenoxam, mefenpyr, mefentrifluconazole, mefluidide, megatomoic acid, melissyl alcohol, melitoxin, MEMC, menazon, MEP, mepanipyrim, meperfluthrin, mephenate, mephosfolan, mepiquat, mepitriflufenpyr, mepronil, meptyldinocap, mercaptodimethur, mercaptophos, mercaptophos thiol, mercaptothion, mercuric chloride, mercuric oxide, mercurous chloride, merphos, merphos oxide, mesoprazine, mesosulfuron, mesotrione, mesulfen, mesulfenfos, mesulphen, metacresol, metaflumizone, metalaxyl, metalaxyl-M, R-metalaxyl, metaldehyde, metam, metamifop, metamitron, metaphos, metarylpicoxamid, metaxon, metazachlor, metazosulfuron, metazoxolon, metcamifen, metconazole, metepa, metflurazon, methabenzthiazuron, methacrifos, methalpropalin, metham, methamidophos, methasulfocarb, methazole, methfuroxam, methibenzuron, methidathion, methiobencarb, methiocarb, methiopyrisulfuron, methiotepa, methiozolin, methiuron, methocrotophos, métholcarb, methometon, methomyl, methoprene, S-methoprene, methoprotryn, methoprotryne, methoquin-butyl, methothrin, methoxychlor, 2-methoxyethylmercury chloride, methoxyfenozide, methoxyphenone, methyl apholate, methyl bromide, methyl eugenol, methyl iodide, methyl-isofenphos, methyl isothiocyanate, methyl parathion, methylacetophos, methylchloroform, 1-methylcyclopropene, methyldithiocarbamic acid, methyldymron, methylene chloride, methylmercaptophos, methylmercaptophos oxide, methylmercaptophos thiol, methylmercury benzoate, methylmercury dicyandiamide, methylmercury pentachlorophenoxide, methylneodecanamide, methylnitrophos, methyltriazothion, metiozolin, metiram, metiram-zinc, metobenzuron, metobromuron, metofluthrin, epsilon-metofluthrin, metolachlor, S-metolachlor, metolcarb, metomeclan, metometuron, metominostrobin, metosulam, metoxadiazone, metoxuron, metrafenone, metriam, metribuzin, metrifonate, metriphonate, metsulfovax, metsulfuron, metyltetraprole, mevinphos, mexacarbate, miechuwei, mieshuan, miewenjuzhi, milbemectin, milbemycin oxime, milneb, mimanan, mipafox, MIPC, mirex, MITC, mivorilaner, MNAF, modoflaner, moguchun, molinate, molosultap, momfluorothrin, epsilon-momfluorothrin, monalide, monisouron, monisuron, monoamitraz, monochloroacetic acid, monocrotophos, monolinuron, monomehypo, monosulfiram, monosulfuron, monosulfuron-ester, monosultap, monuron, monuron-TCA, morfamquat, moroxydine, morphothion, morzid, moxidectin, MPMC, MSMA, MTMC, α-multistriatin, muscalure, myclobutanil, myclozolin, myricyl alcohol, NAA, NAAm, nabam, naftalofos, naled, naphthalene, naphthaleneacetamide, α-naphthaleneacetic acids, naphthalic anhydride, naphthalophos, 1-naphthol, naphthoxyacetic acids, naphthylacetic acids, naphthylindane-1,3-diones, naphthyloxyacetic acids, naproanilide, napropamide, napropamide-M, naptalam, natamycin, NBPOS, neburea, neburon, nendrin, neonicotine, nichlorfos, niclofen, niclosamide, nicobifen, nicofluprole, nicosulfuron, nicotine, nifluridide, nikkomycins, ningnamycin, ningnanmycin, NIP, nipyraclofen, nipyralofen, nitenpyram, nithiazine, nitralin, nitrapyrin, nitrilacarb, nitrofen, nitrofluorfen, nitrostyrene, nitrothal-isopropyl, NNM, nobormide, nonanol, norbormide, norea, norflurazon, nornicotine, noruron, novaluron, noviflumuron, NPA, nuarimol, nuranone, OCH, octachlorodipropyl ether, octhilinone, 2-(octylthio) ethanol, o-dichlorobenzene, ofurace, omethoate, o-phenylphenol, orbencarb, orfralure, orthobencarb, ortho-dichlorobenzene, orthonil, orthosulfamuron, oryctalure, orysastrobin, oryzalin, osthol, osthole, ostramone, ovatron, ovex, oxabetrinil, oxadiargyl, oxadiazon, oxadixyl, oxamate, oxamyl, oxapyrazon, oxapyrazone, oxasulfuron, oxathiapiprolin, oxaziclomefone, oxazosulfyl, oxine-copper, oxine-Cu, oxolinic acid, oxpoconazole, oxycarboxin, oxydemeton-methyl, oxydeprofos, oxydisulfoton, oxyenadenine, oxyfenthiin, oxyfenthin, oxyfluorfen, oxymatrine, oxytetracycline, oxythioquinox, PAC, paclobutrazol, paichongding, palléthrine, PAP, para-dichlorobenzene, parafluron, paraquat, parathion, parathion-methyl, parinol, Paris green, PCNB, PCP, PCP-Na, p-dichlorobenzene, PDJ, pebulate, pédinex, pefurazoate, pelargonic acid, penconazole, pencycuron, pendimethalin, penfenate, penflufen, penfluron, penoxalin, penoxsulam, pentachlorophenol, pentachlorophenyl laurate, pentanochlor, penthiopyrad, pentmethrin, pentoxazone, perbutin, perchlordecone, perfluidone, permethrin, perthane, pethoxamid, PHC, phenamacril, phenamacril-ethyl, phénaminosulf, phenazine oxide, phénétacarbe, phenisopham, phenkapton, phenmedipham, phenmedipham-ethyl, phenobenzuron, phenothiol, phenothrin, phenproxide, phenthoate, 8-phenylmercurioxyquinoline, phenylmercuriurea, phenylmercury acetate, phenylmercury chloride, phenylmercury derivative of pyrocatechol, phenylmercury nitrate, phenylmercury salicylate, 2-phenylphenol, phorate, phosacetim, phosalone, phosametine, phosazetim, phosazetin, phoscyclotin, phosdiphen, phosethyl, phosfolan, phosfolan-methyl, phosglycin, phosmet, phosnichlor, phosphamide, phosphamidon, phosphine, phosphinothricin, phosphocarb, phosphorus, phostebupirim, phostin, phoxim, phoxim-methyl, phthalide, phthalophos, phthalthrin, picarbutrazox, picaridin, picloram, picolinafen, picoxystrobin, pimaricin, pindone, pinoxaden, piperalin, piperazine, piperonyl butoxide, piperonyl cyclonene, piperophos, piproctanly, piproctanyl, piprotal, pirimetaphos, pirimicarb, piriminil, pirimioxyphos, pirimiphos-ethyl, pirimiphos-methyl, pival, pivaldione, plifenate, PMA, PMP, polybutenes, polycarbamate, polychlorcamphene, polyethoxyquinoline, polyoxin D, polyoxins, polyoxorim, polyram, polythialan, potassium arsenite, potassium azide, potassium cyanate, potassium ethylxanthate, potassium naphthenate, potassium polysulfide, potassium thiocyanate, pp′-DDT, prallethrin, precocene I, precocene II, precocene III, pretilachlor, primidophos, primisulfuron, probenazole, prochloraz, proclonol, procyazine, procymidone, prodiamine, profenofos, profluazol, profluralin, profluthrin, profoxydim, profurite-aminium, proglinazine, prohexadione, prohydrojasmon, promacyl, promecarb, prometon, prometryn, prometryne, promurit, pronitridine, pronamide, propachlor, propafos, propamidine, propamocarb, propanil, propaphos, propaquizafop, propargite, proparthrin, propazine, propetamphos, propham, propiconazole, propidine, propineb, propisochlor, propoxur, propoxycarbazone, propyl isome, propyrisulfuron, propyzamide, proquinazid, prosuler, prosulfalin, prosulfocarb, prosulfuron, prothidathion, prothiocarb, prothioconazole, prothiofos, prothoate, protrifenbute, proxan, prymidophos, prynachlor, psoralen, psoralene, pydanon, pydiflumetofen, pyflubumide, pymetrozine, pyracarbolid, pyraclofos, pyraclonil, pyraclostrobin, pyraflufen, pyrafluprole, pyramat, pyrametostrobin, pyraoxystrobin, pyrapropoyne, pyrasulfotole, pyraziflumid, pyrazolate, pyrazolynate, pyrazon, pyrazophos, pyrazosulfuron, pyrazothion, pyrazoxyfen, pyresmethrin, pyrethrin I, pyrethrin II, pyrethrins, pyribambenz-isopropyl, pyribambenz-propyl, pyribencarb, pyribenzoxim, pyributicarb, pyriclor, pyridaben, pyridachlometyl, pyridafol, pyridalyl, pyridaphenthion, pyridaphenthione, pyridate, pyridinitril, pyrifenox, pyrifluquinazon, pyriftalid, pyrimétaphos, pyrimethanil, pyrimicarbe, pyrimidifen, pyriminobac, pyriminostrobin, pyrimiphos-éthyl, pyrimiphos-méthyl, pyrimisulfan, pyrimitate, pyrinuron, pyriofenone, pyriprole, pyripropanol, pyriproxyfen, pyrisoxazole, pyrithiobac, pyrolan, pyroquilon, pyroxasulfone, pyroxsulam, pyroxychlor, pyroxyfur, qincaosuan, qingkuling, quassia, quinacetol, quinalphos, quinalphos-methyl, quinazamid, quinclorac, quinconazole, quinmerac, quinoclamine, quinofumelin, quinomethionate, quinonamid, quinothion, quinotrione, quinoxyfen, quintiofos, quintozene, quintrione, quizalofop, quizalofop-P, quwenzhi, quyingding, rabenzazole, rafoxanide, R-diniconazole, rebemide, reglone, renofluthrin, renriduron, rescalure, resmethrin, d-trans-resmethrin, rhodethanil, rhodojaponin-III, ribavirin, rimisoxafen, rimsulfuron, rizazole, R-metalaxyl, rodéthanil, ronnel, rotenone, ryania, sabadilla, saflufenacil, saijunmao, saisentong, salicylanilide, salifluofen, sanguinarine, santonin, sarolaner, S-bioallethrin, schradan, scilliroside, seboctylamine, sebuthylazine, secbumeton, sedaxane, selamectin, semiamitraz, sesamex, sesamolin, sesone, sethoxydim, sevin, S-hydroprene, shuangjiaancaolin, shuangjianancaolin, siduron, sifumijvzhi, siglure, silafluofen, silatrane, silica aerogel, silica gel, silthiofam, silthiopham, silthiophan, silvex, simazine, simeconazole, simeton, simetryn, simetryne, sintofen, S-kinoprene, slaked lime, SMA, S-methoprene, S-metolachlor, sodium arsenite, sodium azide, sodium chlorate, sodium chloroacetate, sodium cyanide, sodium fluoride, sodium fluoroacetate, sodium hexafluorosilicate, sodium naphthenate, sodium orthophenylphenoxide, sodium pentachlorophenate, sodium pentachlorophenoxide, sodium o-phenylphenoxide, sodium polysulfide, sodium silicofluoride, disodium tetraborate, sodium tetrathiocarbonate, sodium thiocyanate, solan, sophamide, spidoxamat, spinetoram, spinosad, spirobudifen, spirodiclofen, spiromesifen, spiropidion, spirotetramat, spiroxamine, stirofos, streptomycin, strychnine, sulcatol, sulcofuron, sulcotrione, sulfallate, sulfathiadiazuron, sulfathiazuron, sulfentrazone, sulfiram, sulfluramid, sulfodiazole, sulfometuron, sulfosate, sulfosulfuron, sulfotep, sulfotepp, sulfoxaflor, sulfoxide, sulfoxime, sulfur, sulfuric acid, sulfuryl fluoride, sulglycapin, sulphosate, sulprofos, sultropen, suthiazuron, supermethrin, swep, 2,4,5-T, tartar emetic, tau-fluvalinate, tavron, tazimcarb, 2,4,5-TB, 2,3,6-TBA, TBTO, TBZ, TCA, TCBA, TCMTB, TCNB, TDE, tebuconazole, tebufenozide, tebufenpyrad, tebufloquin, tebupirimfos, tebupirimphos, tebutam, tebuthiuron, tecloftalam, tecnazene, tecoram, tedion, teflubenzuron, tefluthrin, kappa-tefluthrin, d-teflumethrin, tefuryltrione, tembotrione, temefos, temephos, tepa, TEPP, tepraloxydim, teproloxydim, terallethrin, terbacil, terbucarb, terbuchlor, terbuconazole, terbufos, terbumeton, terbuthylazine, terbutol, terbutrazole, terbutryn, terbutryne, terraclor, terramicin, terramycin, tetcyclacis, tetflupyrolimet, tetrachlorantraniliprole, tetrachloroethane, tetrachlorvinphos, tetraconazole, tetradifon, tetradisul, tetrafluron, tetramethrin, tetramethylfluthrin, tetramine, tetranactin, tetraniliprole, tetrapion, tetrasul, thallium sulfate, thallous sulfate, thenylchlor, theta-cypermethrin, thiabendazole, thiacloprid, thiadiazine, thiadifluor, thiamethoxam, thiameturon, thiapronil, thiazafluron, thiazfluron, thiazone, thiazopyr, thicrofos, thicyofen, thidiazimin, thidiazuron, thiencarbazone, thifensulfuron, thifluzamide, thimerosal, thimet, thiobencarb, thiocarboxime, thiochlorfenphim, thiocyanatodinitrobenzenes, thiocyclam, thiodan, thiodemeton, thiodiazole-copper, thiodicarb, thiofanocarb, thiofanox, thiofluoximate, thiohempa, thiomersal, thiometon, thionazin, thiophanate, thiophanate-ethyl, thiophanate-methyl, thiophos, thioquinox, thiosemicarbazide, thiosultap, thiotepa, thioxamyl, thiram, thiuram, thuringiensin, tiabendazole, tiadinil, tiafenacil, tiaojiean, TIBA, tifatol, tigolaner, tiocarbazil, tioclorim, tiorantraniliprole, tioxazafen, tioxymid, TMTD, tirpate, tolclofos-methyl, tolfenpyrad, tolnifanide, tolprocarb, tolpyralate, tolyfluanid, tolylfluanid, tolylmercury acetate, tomarin, topramezone, toxaphene, 2,4,5-TP, 2,3,3-TPA, TPN, tralkoxydim, tralocythrin, tralomethrin, tralopyril, d-trans-allethrin, d-trans-resmethrin, transfluthrin, transpermethrin, tretamine, tri-allate, triacontanol, triadimefon, triadimenol, triafamone, triallate, triamiphos, triapenthenol, triarathene, triarimol, triasulfuron, triazamate, triazbutil, triaziflam, triazofenamide, triazophos, triazothion, triazoxide, tribasic copper chloride, tribasic copper sulfate, tribenuron, tribufos, tributyltin oxide, tricamba, trichlamide, trichlophenidine, trichlopyr, trichlorfon, trichlormetaphos-3, trichloronat, trichloronate, trichlorotrinitrobenzenes, trichlorphon, triclopyr, triclopyricarb, tricresol, tricyclazole, tricyclohexyltin hydroxide, tridemorph, tridiphane, trietazine, trifenmorph, trifenofos, trifloxystrobin, trifloxysulfuron, trifludimoxazin, trifluenfuronate, triflumezopyrim, triflumizole, triflumuron, trifluralin, triflusulfuron, trifop, trifopsime, triforine, trihydroxytriazine, 2,3,5-tri-iodobenzoic acid, 2,3,5-triiodobenzoic acid, trimedlure, trimefluor, trimethacarb, trimeturon, trimorfamid, trimorphamide, trinexapac, triphenyltin, triprene, tripropindan, triptolide, tripyrasulfone, tritac, trithialan, triticonazole, tritosulfuron, tropital, trunc-call, tuoyelin, tyclopyrazoflor, umifoxolaner, uniconazole, uniconazole-P, urbacide, uredepa, valerate, validamycin, validamycin A, valifenalate, valone, vamidothion, vangard, vaniliprole, verbutin, vernolate, vinclozolin, viniconazole, vitamin D3, warfarin, xiaochongliulin, xinjunan, xiwojunan, xiwojunzhi, XMC, xylachlor, xylenols, xyloxadine, xylylcarb, xymiazole, yishijing, zarilamid, zeatin, zengxiaoan, zengxiaolin, zeta-cypermethrin, zinc naphthenate, zinc phosphide, zinc thiazole, zinc thiozole, zinc trichlorophenate, zinc trichlorophenoxide, zineb, ziram, zolaprofos, zoocoumarin, zoxamide, zuoanjunzhi, zuocaoan, zuojunzhi, zuomihuanglong, 1-MCP, 1-methylcyclopropene, 1-naphthol, 1,2-dichloropropane, 1,3-D, 1,3-dichloropropene, 2iP, 2M-4C, 2M-4 CM, 2-methoxyethylmercury chloride, 2-(octylthio) ethanol, 2-phenylphenol, 2,2,3-TPA, 2,3,5-triiodobenzoic acid, 2,3,6-TBA, 2,4-D, 2,4-DB, 2,4-DEB, 2,4-DEP, 2,4-DES, 2,4-DP, 2,4-MCPA, 2,4-MCPB, 2,4,5-T, 2,4,5-TB, 2,4,5-TP, 2,5-dichlorobenzoic acid, (3-ethoxypropyl) mercury bromide, 3,4-DA, 3,4-DB, 3,4-DP, 3,6-dichloropicolinic acid, 4-aminopyridine, 4-CPA, 4-CPB, 4-CPP, 4-hydroxyphenethyl alcohol, 8-hydroxyquinoline sulfate, 8-phenylmercurioxyquinoline, and 24-epibrassinolide.

As used in this disclosure, each of the above is an active ingredient. For more information consult the materials listed in the “Compendium of Pesticide Common Names,” located at https://pesticidecompendium.bcpc.org.

A particularly preferred selection of active ingredients are chlorantraniliprole, cyantraniliprole, hexaflumuron, methomyl, methoxyfenozide, noviflumuron, oxamyl, spinetoram, spinosad, sulfoxaflor, and triflumezopyrim (hereafter “AIGA-2”).

Additionally, another particularly preferred selection of active ingredients are acequinocyl, acetamiprid, acetoprole, avermectin, azinphos-methyl, bifenazate, bifenthrin, carbaryl, carbofuran, chlorfenapyr, chlorfluazuron, chromafenozide, clothianidin, cyfluthrin, cypermethrin, deltamethrin, diafenthiuron, emamectin benzoate, endosulfan, esfenvalerate, ethiprole, etoxazole, fipronil, flonicamid, fluacrypyrim, gamma-cyhalothrin, halofenozide, indoxacarb, lambda-cyhalothrin, lufenuron, malathion, methomyl, novaluron, permethrin, pyridalyl, pyrimidifen, spirodiclofen, tebufenozide, thiacloprid, thiamethoxam, thiodicarb, tolfenpyrad, and zeta-cypermethrin (hereafter “AIGA-3”).

Seed treatments are used alone or in combination to address or prevent a number of pests, diseases, nutrient deficiencies, and to enhance plant growth. These seed treatments may include fungicides, insecticides, inoculants, plant growth regulators, fertilizers, and fertilizer enhancers. Currently, the following fungicides may be used with the polymorph Forms A and B of Compound 1 (disclosed herein) (R)-flutriafol, (R)-hexaconazole, (S)-flutriafol, (S)-hexaconazole, 10,10′-oxybisphenoxarsine, 2-(thiocyanomethylthio) benzothiazole, 2,2-dibromo-3-nitrilopropionamide, 2,4,5-trichlorophenol, 2,4-dimethylphenol, 2,5-dichlorobenzoic acid methyl ester, 2,6-dichloro-N-((4-(trifluoromethyl) phenyl) methyl-benzamide, 24-epibrassinolide, 2-allyphenol, 2-aminobutane, 2-methoxyethylmercury acetate, 2-methoxyethylmercury chloride, 2-phenylphenol, 8-hydroxyquinoline, Acibenzolar-S-methyl, Aldimorph, Ametoctradin, Amisulbrom, Ammonium acetate, Ammonium carbonate, Ampropylfos, Anilazine, Anthracene oil, Asomate, Azaconazole, Azithiram, Azoxystrobin, Barium polysulphide, Benalaxyl, Benalaxyl-M, Benodanil, Benomyl, Benquinox, Bentaluron, Benthiavalicarb, Benthiavalicarb isopropyl, Benzalkonium chloride, Benzamacril, Benzamacril isobutyl, Benzamorf, Benzoic acid, Benzovindiflupyr, Bethoxazin, Binapacryl, Biphenyl, Bis (methylmercury) sulphate, Bismerthiazol, Bis-trichloromethyl sulfone, Bitertanol, Bithionol, Bixafen, Bordeaux mixture, Boric acid, Boscalid, Bromuconazole, Bronopol, Bupirimate, Buthiobate, Calcium carbonate, Calcium chloride, Calcium cyanamide, Calcium hydroxide, Calcium phosphate, Captafol, Captan, Carbamorph, Carbendazim, Carboxin, Carpropamid, Chinomethionat, Chlobenthiazone, Chloraniformethan, Chloranil, Chlordecone, Chlorfenazole, Chloroneb, Chlorothalonil, Chloroxylenol, Chlorquinox, Chlozolinate, Cis-propiconazole, Climbazole, Copper (1) oxide, Copper abietate, Copper bis (3-phenylsalicylate), Copper II acetate, Copper II carbonate, Copper II chloride, Copper II hydroxide, Copper naphthenate, Copper oxychloride, Copper sulphate, COS-OGA, Coumethoxystrobin, Coumoxystrobin, Cufraneb, Cuprobam, Cyazofamid, Cycloheximide, Cyflufenamid, Cymoxanil, Cypendazole, Cyproconazole, Cyprodinil, Cyprofuram, Dazomet, D-D, Debacarb, Decafentin, Dehydroacetic acid, Diammonium ethylenebis (dithiocarbamate), Dibromochloropropane, Dichlobentiazox, Dichlofluanid, Dichlone, Dichlorophen, Diclobutrazol, Diclocymet, Diclomezine, Dicloran, Didecyldimethylammonium chloride, Diethofencarb, Difenoconazole, Difenzoquat, Difenzoquat metilsulfate, Diflumetorim, Dimetachlone, Dimethirimol, Dimethomorph, Dimethyl disulfide, Dimoxystrobin, Diniconazole, Diniconazole-M, Dinobuton, Dinocap, Dinocton, Dinopenton, Dinosulfon, Diphenylamine, Dipymetitrone, Dipyrithione, Disodium octaborate tetrahydrate, Disodium phosphonate, Ditalimfos, Dithianon, DNOC, Dodemorph, Dodemorph acetate, Dodine, Drazoxolon, Edifenphos, Enoxastrobin, Epoxiconazole, Etaconazole, Etem, Ethaboxam, Ethirimol, Ethoxyquin, Ethylene bisisothiocyanate sulphide, Ethylicin, Ethylmercury bromide, Etridiazole, Famoxadone, Fenamidone, Fenaminosulf, Fenaminstrobin, Fenapanil, Fenarimol, Fenbuconazole, Fenfuram, Fenhexamid, Fenitropan, Fenoxanil, Fenpiclonil, Fenpicoxamid, Fenpropidin, Fenpropimorph, Fenpyrazamine, Fentin acetate, Fentin chloride, Fentin hydroxide, Ferbam, Florylpicoxamid, Fluazinam, Flubeneteram, Flubenzimine, Fludioxonil, Flufenoxystrobin, Flumorph, Fluopicolide, Fluopimomide, Fluopyram, Fluoroimide, Fluotrimazole, Fluoxapiprolin, Fluoxastrobin, Fluquinconazole, Flusilazole, Flusulfamide, Flutianil, Flutolanil, Flutriafol, Fluxapyroxad, Folpet, Formaldehyde, Fosetyl, Fosetyl-aluminium, Fuberidazole, Furalaxyl, Furalaxyl-M, Furametpyr, Furconazole, Furconazole-cis, Furfural, Furmecyclox, Furyloxyfen, Gliotoxin, Glutaraldehyde, Glyodin, Griseofulvin, Guazatine, Halacrinate, Hexachlorobenzene, Hexachlorophene, Hexaconazole, Hexylthiofos, Huanjunzuo, Hydrogen peroxide, Hymexazol, Imazalil, Imibenconazole, Iminoctadine, Iminoctadine triacetate, Iminoctadine tris (albesilate), Inezin, Ipconazole, Ipfentrifluconazole, Ipflufenoquin, Iprobenfos, Iprodione, Iprovalicarb, Isobutyric acid, Isofetamid, Isoflucypram, Isopamphos, Isoprothiolane, Isopyrazam, Isotianil, Izopamfos, Kresoxim-methyl, Lime sulphur, Mancopper, Mancozeb, Mandestrobin, Mandipropamid, Maneb, Mebenil, Mecarbinzid, Mefentrifluconazole, Mepanipyrim, Mepronil, Meptyldinocap, Mercuric oxide, Mercurous chloride, Metalaxyl, Metalaxyl-M, Metam-potassium, Metam-sodium, Metazoxolon, Metconazole, Methasulfocarb, Methfuroxam, Methyl isothiocyanate, Methylarsenic sulphide, Methylene bisthiocyanate, Metiram, Metominostrobin, Metrafenone, Metsulfovax, Metyltetraprole, Mucochloric anhydride, Myclobutanil, Myclozolin, N-(3-chloro-2,6-dimethylphenyl)-2-methoxy-N-(tetrahydr-2-oxo-3-furanyl) acetamide, Nabam, Nickel bis (dimethyldithiocarbamate), Niclosamide, Nitrothal isopropyl, Nuarimol, Octhilinone, Ofurace, Orysastrobin, Oxadixyl, Oxathiapiprolin, Oxazosulfyl, Oxine-copper, Oxpoconazole fumarate, Oxycarboxin, Paclobutrazol, Paraffin oil (C11-C25) (4a), Paraffin oil (C11-C30) (4c), Paraffin oil (C15-C30) (4b), Parinol, Penconazole, Pencycuron, Penflufen, Pentachlorophenol, Penthiopyrad, Peroxyacetic acid, Phenyl mercuric acetate, Phenylmercury chloride, Phenylmercury nitrate, Phosdiphen, Phthalide, Picarbutrazox, Picoxystrobin, Piperalin, Potassium bicarbonate, Potassium iodide, Potassium phosphonates, Potassium thiocyanate, Probenazole, Prochloraz, Procymidone, Propamidine, Propamocarb, Propamocarb hydrochloride, Propiconazole, Propineb, Propionic acid, Proquinazid, Prothiocarb, Prothioconazole, Pydiflumetofen, Pyracarbolid, Pyraclostrobin, Pyrametostrobin, Pyraoxystrobin, Pyrapropoyne, Pyraziflumid, Pyrazophos, Pyribencarb, Pyridachlometyl, Pyridinitril, Pyrifenox, Pyrimethanil, Pyrimorph, Pyriofenone, Pyrisoxazole, Pyroquilone, Quinofumelin, Quinoxyfen, Quintozene, Saisentong, Sedaxane, Silthiofam, Simeconazole, Sodium arsenite, Sodium carbonate, Sodium hydrogen carbonate, Sodium hypochlorite, Sodium tetraborate pentahydrate, Spiropidion, Spiroxamine, Sulfuryl fluoride, Sulphur, Tebuconazole, Tebufloquin, Tecloftalam, Tecnazene, Tetraconazole, Thiabendazole, Thicyofen, Thifluzamide, Thiomersal, Thiophanate, Thiophanate-methyl, Thioquinox, Thiram, Tiadinil, Tolclofos-methyl, Tolfenpyrad, Tolprocarb, Tolylfluanid, Trans-propiconazole, Triadimefon, Triadimenol, Triamiphos, Triazoxide, Tributyltin oxide, Trichlamide, Triclopyricarb, Tricyclazole, Tridemorph, Trifloxystrobin, Triflumizole, Triforine, Trioxymethylene, Triticonazole, Urea, Valifenalate, Vinclozolin, Zarilamid, Zinc borate, Zinc oxide, Zineb, Ziram, and Zoxamide, this fungicide group is hereafter “FGK-1.”

Another preferred group of fungicides for use with polymorph Forms A and B of Compound 1 (disclosed herein) in seed treatments is Azoxystrobin, Benomyl, Benzovindiflupyr, Bixafen, Carbendazim, Chlorothalonil, Cymoxanil, Cyproconazole, Dichlobentiazox, Difenoconazole, Ethaboxam, Famoxadone, Fenbuconazole, Fluopyram, Fluindapyr, Fludioxonil, Folpet, Inpyrfluxam, Ipconazole, Ipfentrifluconazole, Isoflucypram, Mancozeb, Maneb, Mefentrifluconazole, Meptyldinocap, Metalaxyl, and Metalaxyl-M (Mefenoxam), Oxathiapiprolin, Penflufen, Picoxystrobin, Prochloraz, Proquinazid, Prothioconazole, Pyraclostrobin, Quinoxyfen, Sedaxane, Thiabendazole, Thiram, Tricyclazole, and Trifloxystrobin, this fungicide group is hereafter “FGK-2.”

The following two fungicide molecules are also preferred to be used with polymorph Forms A and B of Compound 1 (disclosed herein);

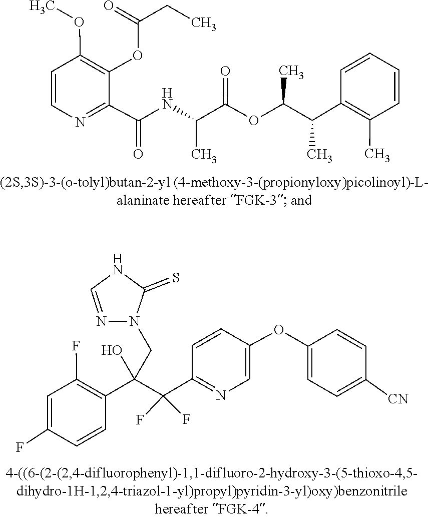 [SMILES: CCC(=O)Oc1c(OC)ccnc1C(=O)N[C@@H](C)C(=O)O[C@@H](C)[C@@H](C)c1ccccc1C ; N#Cc1ccc(Oc2ccc(C(F)(F)C(O)(Cn3nc[nH]c3=S)c3ccc(F)cc3F)nc2)cc1]

FGK-3 described in WO2019173665 as Compound Number 278, and FGK-4 is described in WO2016187201, example 2.

The term “locus” means a habitat, breeding ground, plant, seed, soil, material, or environment, in which a pest is growing, may grow, or may traverse. For example, a locus includes but is not limited to, areas where crops, trees, fruits, cereals, fodder species, vines, turf, and/or ornamental plants, are growing; where domesticated animals are residing; the interior or exterior surfaces of buildings (such as places where grains are stored); in and around materials of construction used in buildings (such as impregnated wood); and the soil around buildings.

The phrase “MoA Material” means an active ingredient having a mode of action (“MoA”) as indicated in IRAC MoA Classification v. 10.3, located at irac-online.org.

The phrase “pesticidally effective amount” means the amount of a pesticide needed to achieve an observable effect on a pest, for example, the effects of necrosis, death, retardation, prevention, removal, destruction, or otherwise diminishing the occurrence and/or activity of a pest in a locus. This effect may come about when pest populations are repulsed from a locus, pests are incapacitated in, or around, a locus, and/or pests are exterminated in, or around, a locus. Of course, a combination of these effects can occur. Generally, pest populations, activity, or both are desirably reduced more than fifty percent, preferably more than 90 percent, and most preferably more than 99 percent, In general, a pesticidally effective amount, for agricultural purposes, is from about 0.0001 grams per hectare to about 5000 grams per hectare, preferably from about 0.0001 grams per hectare to about 500 grams per hectare, and it is even more preferably from about 0.0001 grams per hectare to about 50 grams per hectare.

### SUMMARY

In one aspect, the present disclosure provides one or more crystalline forms of N-[4-chloro-2-(pyridin-3-yl)-1,3-thiazol-5-yl]-N-ethyl-3-(methylsulfonyl) propanamide (Compound 1) represented by the formula

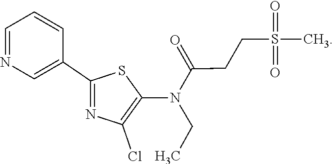 [SMILES: CCN(C(=O)CCS(C)(=O)=O)c1sc(-c2cccnc2)nc1Cl]

In one embodiment, the one or more crystalline forms of N-[4-chloro-2-(pyridin-3-yl) -1,3-thiazol-5-yl]-N-ethyl-3-(methylsulfonyl) propanamide.

In another embodiment, the one or more crystalline forms of N-[4-chloro-2-(pyridin-3-yl)-1,3-thiazol-5-yl]-N-ethyl-3-(methylsulfonyl) propanamide are anhydrous and solvent-free crystalline polymorph forms.

In another embodiment, the one or more crystalline forms are crystalline polymorph Forms A and B (individually referred to herein as polymorph Form A and polymorph Form B) of Compound 1.

In a further embodiment, the crystalline polymorph Form A of Compound 1 has a powder X-ray diffraction pattern comprising a peak at diffraction angle (2θ) of 20.3±0.2. In a further embodiment, the crystalline polymorph Form A of Compound 1 has a powder X-ray diffraction pattern comprising peaks at diffraction angles (2θ) of 17.4±0.2 and 20.3±0.2. In a further embodiment, the crystalline polymorph Form A of Compound 1 has a powder X-ray diffraction pattern comprising peaks at diffraction angles (2θ) of 17.4±0.2, 19.9±0.2, and −20.3±0.2. In a further embodiment, the crystalline polymorph Form A of Compound 1 has a powder X-ray diffraction pattern comprising peaks at diffraction angles (2θ) of 10.6±0.2, 17.4 ±0.2, 19.9±0.2, and 20.3±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 19.9±0.2, and 20.3±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 18.7±0.7, 19.9 ±0.2, and 20.3±0.2. In a further embodiment, the crystalline polymorph Form A of Compound 1 has a powder X-ray diffraction pattern comprising peaks at diffraction angles (2θ) of 10.6±0.2, 16.0±0.2, 17.4±0.2, 18.2±0.2, 18.7±0.7, 19.9±0.2, and 20.3±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.7, 19.9±0.2, and 20.3±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.7, 18.9±0.2, 19.9±0.2, and 20.3±0.2. In a further embodiment, the crystalline polymorph Form A of Compound 1 has a powder X-ray diffraction pattern comprising peaks at diffraction angles (2θ) of 10.0±0.2, 10.1±0.2, 10.6±0.2, 16.0±0.2, 17.4±0.2, 18.2±0.2, 18.7±0.7, 18.9±0.2, 19.9±0.2, and 20.3±0.2. In a further embodiment, the crystalline polymorph Form A of Compound 1 has a powder X-ray diffraction pattern comprising peaks at diffraction angles (2θ) of 10.0±0.2, 10.1±0.2, 10.6±0.2, 16.0±0.2, 17.4±0.2, 17.9±0.2, 18.2±0.2, 18.7±0.7, 18.9±0.2, 19.9±0.2, and 20.3±0.2.

In a further embodiment, the crystalline polymorph Form A of Compound 1 has a powder X-ray diffraction pattern comprising a peak at diffraction angle (2θ) of 20.3±0.2. In a further embodiment, the crystalline polymorph Form A of Compound 1 has a powder X-ray diffraction pattern comprising peaks at diffraction angles (2θ) of 20.3±0.2 and 24.7±0.2. In a further embodiment, the crystalline polymorph Form A of Compound 1 has a powder X-ray diffraction pattern comprising peaks at diffraction angles (2θ) of 17.4±0.2, 20.3±0.2, and −24.7±0.2. In a further embodiment, the crystalline polymorph Form A of Compound 1 has a powder X-ray diffraction pattern comprising peaks at diffraction angles (2θ) of 17.4±0.2, 20.3 ±0.2, 24.7±0.2, and 26.6±0.2. In a further embodiment, the crystalline polymorph Form A of Compound 1 has a powder X-ray diffraction pattern comprising peaks at diffraction angles (2θ) of 17.4±0.2, 19.9±0.2, 20.3±0.2, 24.7±0.2, and 26.6±0.2. In a further embodiment, the crystalline polymorph Form A of Compound 1 has a powder X-ray diffraction pattern comprising peaks at diffraction angles (2θ) of 10.6±0.2, 17.4±0.2, 19.9±0.2, 20.3±0.2, 24.7 ±0.2, and 26.6±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 19.9±0.2, 20.3±0.2, 24.7±0.2, and 26.6±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 19.9±0.2, 20.3±0.2, 24.7±0.2, 26.6±0.2, and 28.1±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 19.9±0.2, 20.3±0.2, 24.7±0.2, 25.3±0.2, 26.6±0.2, and 28.1±0.2. In a further embodiment, the crystalline polymorph Form A of Compound 1 has a powder X-ray diffraction pattern comprising peaks at diffraction angles (2θ) of 10.6±0.2, 17.4±0.2, 18.2±0.2, 18.7±0.2, 19.9±0.2, 20.3±0.2, 24.7±0.2, 25.3±0.2, 26.6±0.2, and 28.1±0.2. In a further embodiment, the crystalline polymorph Form A of Compound 1 has a powder X-ray diffraction pattern comprising peaks at diffraction angles (2θ) of 10.6±0.2, 16.0±0.2, 17.4±0.2, 18.2±0.2, 18.7±0.2, 19.9±0.2, 20.3±0.2, 24.7±0.2, 25.3±0.2, 26.6±0.2, and 28.1±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.2, 19.9±0.2, 20.3±0.2, 24.7±0.2, 25.3±0.2, 26.6±0.2, and 28.1±0.2.

In a further embodiment, the crystalline polymorph Form A of Compound 1 has a powder X-ray diffraction pattern comprising peaks at diffraction angles (2θ) of 10.1±0.2, 10.6 ±0.2, 16.0±0.2, 17.4±0.2, 18.2±0.2, 18.7±0.2, 19.9±0.2, 20.3±0.2, 21.0±0.2, 24.7±0.2, 25.3±0.2, 26.6±0.2, and 28.1±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.2, 19.9±0.2, 20.3±0.2, 21.0±0.2, 24.7±0.2, 25.3±0.2, 26.6±0.2, 28.1±0.2, and 28.6±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.2, 18.9±0.2, 19.9±0.2, 20.3±0.2, 21.0±0.2, 24.7±0.2, 25.3±0.2, 26.6±0.2, 28.1±0.2, and 28.6±0.2. In a further embodiment, the crystalline polymorph Form A of Compound 1 has a powder X-ray diffraction pattern comprising peaks at diffraction angles (2θ) of 10.1±0.2, 10.6±0.2, 16.0±0.2, 17.4±0.2, 18.2±0.2, 18.7±0.2, 18.9±0.2, 19.9±0.2, 20.3±0.2, 21.0±0.2, 23.9±0.2, 24.7±0.2, 25.3±0.2, 26.6±0.2, 28.1±0.2, and 28.6±0.2. In a further embodiment, the crystalline polymorph Form A of Compound 1 has a powder X-ray diffraction pattern comprising peaks at diffraction angles (2θ) of 10.0±0.2, 10.1±0.2, 10.6±0.2, 16.0±0.2, 17.4±0.2, 18.2±0.2, 18.7±0.2, 18.9±0.2, 19.9±0.2, 20.3±0.2, 21.0±0.2, 23.9±0.2, 24.7±0.2, 25.3±0.2, 26.6±0.2, 28.1±0.2, and 28.6±0.2. In a further embodiment, the crystalline polymorph Form A of Compound 1 has a powder X-ray diffraction pattern comprising peaks at diffraction angles (2θ) of 10.0±0.2, 10.1±0.2, 10.6±0.2, 16.0±0.2, 17.4±0.2, 17.9±0.2, 18.2±0.2, 18.7±0.2, 18.9±0.2, 19.9±0.2, 20.3±0.2, 21.0±0.2, 23.9±0.2, 24.7±0.2, 25.3±0.2, 26.6±0.2, 28.1±0.2, and 28.6±0.2.

In a further embodiment, the crystalline polymorph Form A of Compound 1 has a powder X-ray diffraction pattern comprising peaks at diffraction angles (2θ) essentially the same as shown in FIG. 1 or FIG. 2.

In a further embodiment, the crystalline polymorph form A of Compound 1 has a Differential Scanning calorimetry (DSC) thermogram comprising an endothermal peak having a peak temperature at about 101.09° C. and a heat of Fusion=75.707 J/g or substantially the same as FIG. 4.

In a further embodiment, the crystalline polymorph form A of Compound 1 has a low frequency Raman spectrum comprising one or more peaks at wavenumbers of about 255 cm⁻¹, about 441 cm⁻¹, about 539 cm⁻¹, about 778 cm⁻¹, about 921 cm⁻¹, about 991 cm⁻¹, about 1048 cm⁻¹, about 1123 cm⁻¹, about 1191 cm⁻¹, about 1526 cm⁻¹, about 1569 cm⁻¹ about 1588 cm⁻¹ about 1701 cm⁻¹, about 2949 cm⁻¹ and about 3053 cm⁻¹. In a further embodiment, the crystalline polymorph form A of Compound 1 has a low frequency Raman spectrum comprising peaks at wavenumbers essentially the same as shown in FIG. 6.

In some embodiments in connection with any of the powder X-ray diffraction patterns for crystalline polymorph form A as described herein, the crystalline polymorph form A further comprises a (DSC) thermogram comprising an endothermal peak having a peak temperature at about 101.09° C., and/or low frequency Raman spectrum comprising one or more peaks at wavenumbers of about 255 cm⁻¹, about 441 cm⁻¹, about 539 cm⁻¹, about 778 cm⁻¹, about 921 cm⁻¹, about 991 cm⁻¹, about 1048 cm⁻¹, about 1123 cm⁻¹, about 1191 cm⁻¹, about 1526 cm⁻¹, about 1569 cm⁻¹ about 1588 cm⁻¹ about 1701 cm⁻¹, about 2949 cm⁻¹ and about 3053 cm⁻¹. In some embodiments in connection with any of the powder X-ray diffraction patterns for crystalline polymorph form A as described herein, the crystalline polymorph form A further comprises a DSC thermogram substantially the same as FIG. 4 and/or a low frequency Raman spectrum comprising peaks at wavenumbers essentially the same as shown in FIG. 6.

In a further embodiment, the crystalline polymorph Form B of Compound 1 has a powder X-ray diffraction pattern comprising a peak at diffraction angle (2θ) of 15.4±0.2. In a further embodiment, the crystalline polymorph Form B of Compound 1 has a powder X-ray diffraction pattern comprising peaks at diffraction angles (2θ) of 7.7±0.2 and 15.4±0.2. In a further embodiment, the crystalline polymorph Form B of Compound 1 has a powder X-ray diffraction pattern comprising peaks at diffraction angles (2θ) of 7.7±0.2, 15.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, and 20.2±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, and 20.2±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 18.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.4±0.2, 16.6±0.2, 16.7±0.2, 17.5±0.2, 18.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.5±0.2, 18.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.4±0.2, 19.8±0.2, and 20.2±0.2.

In a further embodiment, the crystalline polymorph Form B of Compound 1 has a powder X-ray diffraction pattern comprising a peak at diffraction angle (2θ) of 15.4±0.2. In a further embodiment, the crystalline polymorph Form B of Compound 1 has a powder X-ray diffraction pattern comprising peaks at diffraction angles (2θ) of 7.7±0.2 and 15.4±0.2. In a further embodiment, the crystalline polymorph Form B of Compound 1 has a powder X-ray diffraction pattern comprising peaks at diffraction angles (2θ) of 7.7±0.2, 15.4±0.2, and 20.2±0.2. In a further embodiment, the crystalline polymorph Form B of Compound 1 has a powder X-ray diffraction pattern comprising peaks at diffraction angles (2θ) of 7.7±0.2, 15.4±0.2, 20.2±0.2, and 24.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 20.2±0.2, and 24.1±0.2. In a further embodiment, the crystalline polymorph Form A of Compound 1 has a powder X-ray diffraction pattern comprising peaks at diffraction angles (2θ) of 7.7±0.2, 15.4±0.2, 16.7±0.2, 20.2±0.2, 24.1±0.2, and 31.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 20.2±0.2, 24.1±0.2, 26.1±0.2, and 31.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 20.2±0.2, 24.1±0.2, 26.1±0.2, and 31.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 20.2±0.2, 24.1±0.2, 26.1±0.2, 26.6±0.2, and 31.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 20.2±0.2, 24.1±0.2, 26.1±0.2, 26.6±0.2, 27.0±0.2, and 31.1±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 20.2±0.2, 24.1±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 18.5±0.2, 20.2±0.2, 24.1±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.4±0.2, 16.7±0.2, 17.5±0.2, 18.5±0.2, 20.2±0.2, 21.8±0.2, 24.1±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.4±0.2, 16.6±0.2, 16.7±0.2, 17.5±0.2, 18.5±0.2, 20.2±0.2, 21.8±0.2, 24.1±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.4±0.2, 16.6±0.2, 16.7±0.2, 17.5±0.2, 18.5±0.2, 20.2±0.2, 21.8±0.2, 24.1±0.2, 25.8±0.2, 26.1±0.2, 26.6±0.2, 27.0±0.2, 31.1±0.2, and 33.4±0.2.

In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.5±0.2, 18.5±0.2, 20.2±0.2, 21.8±0.2, 24.1±0.2, 25.8±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.5±0.2, 18.5±0.2, 20.2±0.2, 20.9±0.2, 21.8±0.2, 24.1±0.2, 25.8±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7 ±0.2, 17.5±0.2, 18.5±0.2, 20.2±0.2, 20.9±0.2, 21.8±0.2, 22.9±0.2, 24.1±0.2, 25.8±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7 ±0.2, 17.3±0.2, 17.5±0.2, 18.5±0.2, 20.2±0.2, 20.9±0.2, 21.8±0.2, 22.9±0.2, 24.1±0.2, 25.8±0.2, 26.1±0.2, 26.6±0.2, 27.0±0.2, 31.1±0.2, and 33.4±0.2. In a further embodiment, the crystalline polymorph Form B of Compound 1 has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.5±0.2, 19.8±0.2, 20.2±0.2, 20.9±0.2, 21.8±0.2, 22.9±0.2, 24.1±0.2, 25.8±0.2, 26.1±0.2, 26.6±0.2, 27.0±0.2, 31.1±0.2, and 33.4±0.2.

In a further embodiment, the crystalline polymorph Form B of Compound 1 has a powder X-ray diffraction pattern comprising peaks at diffraction angles (2θ) essentially the same as shown in FIG. 3.

In a further embodiment, the crystalline polymorph form B of Compound 1 has a Differential Scanning calorimetry (DSC) thermogram comprising an endothermal peak having a peak temperature at about 105.24° C. and a heat of Fusion=92.879 J/g or substantially the same as FIG. 5.

In a further embodiment, the crystalline polymorph form B of Compound 1 has a low frequency Raman spectrum comprising one or more peaks at wavenumbers of about 266 cm⁻¹, about 446 cm⁻¹, about 546 cm⁻¹, about 763 cm⁻¹, about 987 cm⁻¹, about 1044 cm⁻¹, about 1137 cm⁻¹, about 1187 cm⁻¹ and about 1308 cm⁻¹, about 1518 cm⁻¹, about 1573 cm⁻¹, about 1592 cm⁻¹, about 1673 cm⁻¹, about 2919 cm⁻¹, and about 2937 cm⁻¹. In a further embodiment, the crystalline polymorph form B of Compound 1 has a low frequency Raman spectrum comprising peaks at wavenumbers essentially the same as shown in FIG. 7.

In some embodiments in connection with any of the powder X-ray diffraction patterns for crystalline polymorph form B as described herein, the crystalline polymorph form B further comprises a (DSC) thermogram comprising an endothermal peak having a peak temperature at about 105.24° C. and/or low frequency Raman spectrum comprising one or more peaks at wavenumbers of about 266 cm⁻¹, about 446 cm⁻¹, about 546 cm⁻¹, about 763 cm⁻¹, about 987 cm⁻¹, about 1044 cm⁻¹, about 1137 cm⁻¹, about 1187 cm⁻¹ and about 1308 cm⁻¹, about 1518 cm⁻¹, about 1573 cm⁻¹, about 1592 cm⁻¹, about 1673 cm⁻¹, about 2919 cm⁻¹, and about 2937 cm⁻¹. In some embodiments in connection with any of the powder X-ray diffraction patterns for crystalline polymorph form B as described herein, the crystalline polymorph form B further comprises a DSC thermogram substantially the same as FIG. 5 and/or a low frequency Raman spectrum comprising peaks at wavenumbers essentially the same as shown in FIG. 7.

The present disclosure further provides a composition comprising one or more of polymorph Forms A and B of Compound 1.

In another aspect, the disclosure provides a process to control a pest said process comprising applying to a locus, a pesticidally effective amount of one or more of polymorph Forms A and B of N-[4-chloro-2-(pyridin-3-yl)-1,3-thiazol-5-yl]-N-ethyl-3-(methylsulfonyl) propanamide, as described herein, or a composition comprising one or more of polymorph Forms A and B of N-[4-chloro-2-(pyridin-3-yl)-1,3-thiazol-5-yl]-N-ethyl-3-(methylsulfonyl) propanamide, as described herein.

In some embodiments of this aspect, said pest is selected from the group consisting of ants, aphids, bed bugs, beetles, bristletails, caterpillars, cockroaches, crickets, earwigs, fleas, flies, grasshoppers, grubs, leafhoppers, lice, locusts, maggots, mites, nematodes, planthoppers, psyllids, sawflies, scales, silverfish, slugs, snails, spiders, springtails, stink bugs, symphylans, termites, thrips, ticks, wasps, whiteflies, and wireworms. In some embodiments, said pest is a sap-feeding pest or a chewing pest.

In some embodiments, said pest is from Order Hemiptera, Thysanoptera, Lepidoptera, and the like.

In some embodiments of this aspect, said pest is selected from the group consisting of Adelges spp., Aulacaspis spp., Aphrophora spp., Aphis spp., Bemisia spp., Ceroplastes spp., Chionaspis spp., Chrysomphalus spp., Coccus spp., Empoasca spp., Euschistus spp., Lepidosaphes spp., Lagynotomus spp., Lygus spp., Macrosiphum spp., Nephotettix spp., Nezara spp., Nilaparvata spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp., and Unaspis spp.

In some embodiments of this aspect, pest is selected from the group consisting of Acrosternum hilare, Acyrthosiphon pisum, Aleyrodes proletella, Aleurodicus dispersus, Aleurothrixus floccosus, Amrasca biguttula biguttula, Aonidiella aurantii, Aphis fabae, Aphis gossypii, Aphis glycines, Aphis pomi, Aulacorthum solani, Bactericera cockerelli, Bagrada hilaris, Bemisia argentifolii, Bemisia tabaci, Blissus leucopterus, Boisea trivittata, Brachycorynella asparagi, Brevennia rehi, Brevicoryne brassicae, Cacopsylla pyri, Cacopsylla pyricola, Calocoris norvegicus, Ceroplastes rubens, Cimex hemipterus, Cimex lectularius, Coccus pseudomagnoliarum, Dagbertus fasciatus, Dichelops furcatus, Diuraphis noxia, Diaphorina citri, Dysaphis plantaginea, Dysdercus suturellus, Edessa meditabunda, Empoasca vitis, Eriosoma lanigerum, Erythroneura elegantula, Eurygaster maura, Euschistus conspersus, Euschistus heros, Euschistus servus, Halyomorpha halys, Helopeltis antonii, Hyalopterus pruni, Helopeltis antonii, Helopeltis theivora, Icerya purchasi, Idioscopus nitidulus, Jacobiasca formosana, Laodelphax striatellus, Lecanium corni, Leptocorisa oratorius, Leptocorisa varicornis, Lygus hesperus, Maconellicoccus hirsutus, Macrosiphum euphorbiae, Macrosiphum granarium, Macrosiphum rosae, Macrosteles quadrilineatus, Mahanarva frimbiolata, Megacopta cribraria, Metopolophium dirhodum, Mictis longicornis, Myzus persicae, Nasonovia ribisnigri, Nephotettix cincticeps, Neurocolpus longirostris, Nezara viridula, Nilaparvata lugens, Paracoccus marginatus, Paratrioza cockerelli, Parlatoria pergandii, Parlatoria ziziphi, Peregrinus maidis, Phylloxera vitifoliae, Physokermes piceae, Phytocoris californicus, Phytocoris relativus, Piezodorus guildinii, Planococcus citri, Planococcus ficus, Poecilocapsus lineatus, Psallus vaccinicola, Pseudacysta perseae, Pseudococcus brevipes, Quadraspidiotus perniciosus, Rhopalosiphum maidis, Rhopalosiphum padi, Saissetia olede, Scaptocoris castanea, Schizaphis graminum, Sitobion avenue, Sogatella furcifera, Trialeurodes vaporariorum, Trialeurodes abutiloneus, Unaspis yanonensis, and Zulia entrerriana.

In some embodiments of this aspect, said pest is selected from the group consisting of Caliothrips spp., Frankliniella spp., Scirtothrips spp., and Thrips spp.

In some embodiments of this aspect, said pest is selected from the group consisting of Caliothrips phaseoli, Frankliniella bispinosa, Frankliniella fusca, Frankliniella occidentalis, Frankliniella schultzei, Frankliniella tritici, Frankliniella williamsi, Heliothrips haemorrhoidalis, Rhipiphorothrips cruentatus, Scirtothrips citri, Scirtothrips dorsalis, Taeniothrips rhopalantennalis, Thrips hawaiiensis, Thrips nigropilosus, Thrips orientalis, Thrips palmi, and Thrips tabaci.

In some embodiments of this aspect, said pest is selected from the group consisting of Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Nemapogon spp., Peridroma spp., Phyllonorycter spp., Pseudaletia spp., Plutella spp., Sesamia spp., Spodoptera spp., Synanthedon spp., and Yponomeuta spp.

In some embodiments of this aspect, said pest is selected from the group consisting of Achaea janata, Adoxophyes orana, Agrotis ipsilon, Alabama argillacea, Amorbia cuneana, Amyelois transitella, Anacamptodes defectaria, Anarsia lineatella, Anomis sabulifera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Corcyra cephalonica, Cossus cossus, Cydia caryana, Cydia funebrana, Cydia molesta, Cydia nigricana, Cydia pomonella, Darna diducta, Diaphania nitidalis, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Estigmene acrea, Eupoecilia ambiguella, Euxoa auxiliaris, Galleria mellonella, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera malifoliella, Lobesia botrana, Loxagrotis albicosta, Lymantria dispar, Lyonetia clerkella, Mahasena corbetti, Mamestra brassicae, Manduca sexta, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Phyllonorycter blancardella, Pieris rapae, Plathypena scabra, Platynota idaeusalis, Plodia interpunctella, Plutella xylostella, Polychrosis viteana, Prays endocarpa, Prays oleae, Pseudaletia unipuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tinea pellionella, Tineola bisselliella, Trichoplusia ni, Tuta absoluta, Zeuzera coffede, and Zeuzea pyrina.

It has been unexpectedly discovered that the crystalline polymorph Forms A and B (individually referred to herein as polymorph Form A and polymorph Form B) of Compound 1 described herein effect the behavior of adult sweet potato whitefly, B. tabaci, nerve and muscle function in a manner consistent with insecticides affecting chordotonal organs previously described herein. In direct comparisons with with the previously known non-crystalline form of Compound 1 (an amorphous oil), it was unexpectedly discovered that the crystalline polymorph Forms A and B (individually referred to herein as polymorph Form A and polymorph Form B) of Compound 1 described herein surprisingly induced a significantly greater knock-down of insects, which has been shown in prior studies of previous insecticides affecting chordotonal organs to relate to mortality.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A crystalline form of N-[4-chloro-2-(pyridin-3-yl)-1,3-thiazol-5-yl]-N-ethyl-3-(methylsulfonyl) propanamide.

2. The crystalline form of embodiment 1, wherein the crystalline form is a crystalline polymorph form of N-[4-chloro-2-(pyridin-3-yl)-1,3-thiazol-5-yl]-N-ethyl-3-(methylsulfonyl) propanamide.

3. The crystalline polymorph of embodiment 1 or 2, wherein the crystalline form is anhydrous or solvent-free.

4. A crystalline polymorph Form A of N-[4-chloro-2-(pyridin-3-yl)-1,3-thiazol-5-yl]-N -ethyl-3-(methylsulfonyl) propanamide having a powder X-ray diffraction pattern comprising a peak at diffraction angle (2θ) of 20.3±0.2.

5. The crystalline polymorph form of embodiment 4, wherein the crystalline polymorph form has a powder X-ray diffraction pattern comprising a peak at diffraction angle (2θ) of 17.4±0.2 and 20.3±0.2.

6. The crystalline polymorph form of embodiment 4 or 5, wherein the crystalline polymorph form has a powder X-ray diffraction pattern comprising peaks at diffraction angles (2θ) of 17.4±0.2, 19.9±0.2, and 20.3±0.2.

7. The crystalline polymorph form of any one of embodiments 4 to 6, wherein the crystalline polymorph form has a powder X-ray diffraction pattern comprising peaks at diffraction angles (2θ) of 10.6±0.2, 17.4±0.2, 19.9±0.2, and 20.3±0.2.

8. The crystalline polymorph form of any one of embodiments 4 to 7, wherein the crystalline polymorph form has a powder X-ray diffraction pattern comprising peaks at diffraction angles (2θ) of 10.6±0.2, 17.4±0.2, 18.2±0.2, 19.9±0.2, and 20.3±0.2.

9. The crystalline polymorph form of any one of embodiments 4 to 8, wherein the crystalline polymorph form has a powder X-ray diffraction pattern comprising peaks at diffraction angles (2θ) of 10.6±0.2, 17.4±0.2, 18.2±0.2, 18.7±0.7, 19.9±0.2, and 20.3±0.2.

10. The crystalline polymorph form of any one of embodiments 4 to 9, wherein the crystalline polymorph form has a powder X-ray diffraction pattern comprising peaks at diffraction angles (2θ) of 10.6±0.2, 16.0±0.2, 17.4±0.2, 18.2±0.2, 18.7±0.7, 19.9±0.2, and 20.3±0.2.

11. The crystalline polymorph form of any one of embodiments 4 to 10, wherein the crystalline polymorph form has a powder X-ray diffraction pattern comprising peaks at diffraction angles (2θ) of 10.1±0.2, 10.6±0.2, 16.0±0.2, 17.4±0.2, 18.2±0.2, 18.7±0.7, 19.9±0.2, and 20.3±0.2.

12. The crystalline polymorph form of any one of embodiments 4 to 11, wherein the crystalline polymorph form has a powder X-ray diffraction pattern comprising peaks at diffraction angles (2θ) of 10.1±0.2, 10.6±0.2, 16.0±0.2, 17.4±0.2, 18.2±0.2, 18.7±0.7, 18.9±0.2, 19.9±0.2, and 20.3±0.2.

13. The crystalline polymorph form of any one of embodiments 4 to 12, wherein the crystalline polymorph form has a powder X-ray diffraction pattern comprising peaks at diffraction angles (2θ) of 10.0±0.2, 10.1±0.2, 10.6±0.2, 16.0±0.2, 17.4±0.2, 18.2±0.2, 18.7±0.7, 18.9±0.2, 19.9±0.2, and 20.3±0.2.

14. The crystalline polymorph form of any one of embodiments 4 to 13, wherein the crystalline polymorph form has a powder X-ray diffraction pattern comprising one or more peaks essentially the same as shown in FIG. 1 or FIG. 2.

15. The crystalline polymorph form of any one of embodiments 4 to 14, wherein the crystalline polymorph form has a DSC thermogram comprising an endothermal peak having a peak temperature at about 101.09° C.

16. The crystalline polymorph form of any one of embodiments 4 to 15, having a DSC thermogram substantially the same as FIG. 4.

17. The crystalline polymorph form of any one of embodiments 4 to 16, having a Raman spectrum comprising one or more peaks at wavenumbers of about 255 cm⁻¹, about 441 cm⁻¹, about 539 cm⁻¹, about 778 cm⁻¹, about 921 cm⁻¹, about 991 cm⁻¹, about 1048 cm⁻¹, about 1123 cm⁻¹, about 1191 cm⁻¹, about 1526 cm⁻¹, about 1569 cm⁻¹ about 1588 cm⁻¹ about 1701 cm⁻¹, about 2949 cm⁻¹ and about 3053 cm⁻¹.

18. The crystalline polymorph form of any one of embodiments 4 to 17, having a low frequency Raman spectrum comprising peaks at wavenumbers essentially the same as shown in FIG. 6.

19. A crystalline polymorph Form B of N-[4-chloro-2-(pyridin-3-yl)-1,3-thiazol-5-yl]-N-ethyl-3-(methylsulfonyl) propanamide having a powder X-ray diffraction pattern comprising a peak at diffraction angle (2θ) of 15.4±0.2.

20. The crystalline polymorph form of embodiment 19, wherein the crystalline polymorph form has a powder X-ray diffraction pattern comprising peaks at diffraction angles (2θ) of 7.7±0.2 and 15.4±0.2.

21. The crystalline polymorph form of embodiment 19 or 20, wherein the crystalline polymorph form has a powder X-ray diffraction pattern comprising peaks at diffraction angles (2θ) of 7.7±0.2, 15.4±0.2, and 20.2±0.2.

22. The crystalline polymorph form of any one of embodiments 19 to 21, wherein the crystalline polymorph form has a powder X-ray diffraction pattern comprising peaks at diffraction angles (2θ) of 7.7±0.2, 15.4±0.2, 16.7±0.2, and 20.2±0.2.

23. The crystalline polymorph form of any one of embodiments 19 to 22, wherein the crystalline polymorph form has a powder X-ray diffraction pattern comprising peaks at diffraction angles (2θ) of 7.7±0.2, 15.4±0.2, 16.7±0.2, 17.5±0.2, and 20.2±0.2.

24. The crystalline polymorph form of any one of embodiments 19 to 23, wherein the crystalline polymorph form has a powder X-ray diffraction pattern comprising peaks at diffraction angles (2θ) of 7.7±0.2, 15.4±0.2, 16.7±0.2, 17.5±0.2, 18.4±0.2, and 20.2±0.2.

25. The crystalline polymorph form of any one of embodiments 19 to 24, wherein the crystalline polymorph form has a powder X-ray diffraction pattern comprising peaks at diffraction angles (2θ) of 7.7±0.2, 15.4±0.2, 16.6±0.2, 16.7±0.2, 17.5±0.2, 18.4±0.2, and 20.2±0.2.

26. The crystalline polymorph form of any one of embodiments 19 to 25, wherein the crystalline polymorph form has a powder X-ray diffraction pattern comprising peaks at diffraction angles (2θ) of 7.7±0.2, 15.0±0.2, 15.4±0.2, 16.6±0.2, 16.7±0.2, 17.5±0.2, 18.4±0.2, and 20.2±0.2.

27. The crystalline polymorph form of any one of embodiments 19 to 26, wherein the crystalline polymorph form has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.4±0.2, and 20.2±0.2.

28. The crystalline polymorph form of any one of embodiments 19 to 27, wherein the crystalline polymorph form has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.4±0.2, 19.8±0.2, and 20.2±0.2.

29. The crystalline polymorph form of any one of embodiments 19 to 28, wherein the crystalline polymorph form has a powder X-ray diffraction pattern comprising one or more peaks essentially the same as shown in FIG. 3.

30. The crystalline polymorph form of any one of embodiments 19 to 29, wherein the crystalline polymorph form has a DSC thermogram comprising an endothermal peak having a peak temperature at about 105.24° C.

31. The crystalline polymorph form of any one of embodiments 19 to 30, having a DSC thermogram substantially the same as FIG. 5.

32. The crystalline polymorph form of any one of embodiments 19 to 31, having a Raman spectrum comprising one or more peaks at wavenumbers of about 266 cm⁻¹, about 446 cm⁻¹, about 546 cm⁻¹, about 763 cm⁻¹, about 987 cm⁻¹, about 1044 cm⁻¹, about 1137 cm⁻¹, about 1187 cm⁻¹ and about 1308 cm⁻¹, about 1518 cm⁻¹, about 1573 cm⁻¹, about 1592 cm⁻¹, about 1673 cm⁻¹, about 2919 cm⁻¹, and about 2937 cm⁻¹.

33. The crystalline polymorph form of any one of embodiments 19 to 32, having a low frequency Raman spectrum comprising peaks at wavenumbers essentially the same as shown in FIG. 7.

34. A composition comprising the crystalline form according to embodiment 1 or the crystalline polymorph form according to any one of embodiments 2 to 33.

35. A process to control a pest said process comprising applying to a locus, a pesticidally effective amount of a crystalline form according to embodiment 1, a crystalline polymorph form according to any one of embodiments 2 to 33, or a composition according to embodiment 34.

### BRIEF DESCRIPTION OF THE DRAWINGS

FIG. 1 shows a powder X-ray diffraction pattern of the crystalline form of N-[4-chloro -2-(pyridin-3-yl)-1,3-thiazol-5-yl]-N-ethyl-3-(methylsulfonyl) propanamide (solvent-free and anhydrous), polymorph Form A, as prepared in Example 1.

FIG. 2 shows a powder X-ray diffraction pattern of the crystalline form of N-[4-chloro -2-(pyridin-3-yl)-1,3-thiazol-5-yl]-N-ethyl-3-(methylsulfonyl) propanamide (solvent-free and anhydrous), polymorph Form A, as prepared in Example 2.

FIG. 3 shows a powder X-ray diffraction pattern of the crystalline form of N-[4-chloro -2-(pyridin-3-yl)-1,3-thiazol-5-yl]-N-ethyl-3-(methylsulfonyl) propanamide (solvent-free and anhydrous), polymorph Form B, as prepared in Example 5.

FIG. 4 shows a differential scanning calorimetry (DSC) thermogram of the crystalline form of N-[4-chloro-2-(pyridin-3-yl)-1,3-thiazol-5-yl]-N-ethyl-3-(methylsulfonyl) propanamide (solvent-free and anhydrous), polymorph Form A.

FIG. 5 shows a differential scanning calorimetry (DSC) thermogram of the crystalline form of N-[4-chloro-2-(pyridin-3-yl)-1,3-thiazol-5-yl]-N-ethyl-3-(methylsulfonyl) propanamide (solvent-free and anhydrous), polymorph Form B.

FIG. 6 shows a Raman spectrum of the crystalline form of N-[4-chloro-2-(pyridin-3-yl) -1,3-thiazol-5-yl]-N-ethyl-3-(methylsulfonyl) propanamide (solvent-free and anhydrous), polymorph Form A.

FIG. 7 shows a Raman spectrum of the crystalline form of N-[4-chloro-2-(pyridin-3-yl) -1,3-thiazol-5-yl]-N-ethyl-3-(methylsulfonyl) propanamide (solvent-free and anhydrous), polymorph Form B.

FIG. 8 is a graph showing the control treatment of a knock-down experiment which records the percent of B. tabaci adults above the 2 cm line in the acetone solvent blank control vials over time.

FIG. 9 is a graph showing the results of a knock-down experiment in which adult white fly, B. tabaci, were placed in vials pre-treated with 25 g/ha of Compound 1, polymorph form B or Compound 1, non-crystalline amorphous oil, each from acetone solvent, and records the percent of B. tabaci adults above the 2 cm line over time in polymorph B vials (●) or non-crystalline amorphous oil vials (▪).

FIG. 10 is a graph showing the results of a knock-down experiment in which adult white fly, B. tabaci, were placed in vials pre-treated with 2.5 g/ha of Compound 1, polymorph form B or Compound 1, non-crystalline amorphous oil, each from acetone solvent, and records the percent of B. tabaci adults above the 2 cm line over time in polymorph B vials (●) or non-crystalline amorphous oil vials (▪).

FIG. 11 is a graph showing the control treatment of a knock-down experiment which records the percent of B. tabaci adults above the 2 cm line in the hexane solvent blank control vials over time.

FIG. 12 is a graph showing the results of a knock-down experiment in which adult white fly, B. tabaci, were placed in vials pre-treated with 25 g/ha of Compound 1, polymorph form A, Compound 1, polymorph form B, or Compound 1, non-crystalline amorphous oil, each from hexane anti-solvent suspension, and records the percent of B. tabaci adults above the 2 cm line over time in polymorph A vials (♦), polymorph B vials (●), or non-crystalline amorphous oil vials (▪).

FIG. 13 is a graph showing the results of a knock-down experiment in which adult white fly, B. tabaci, were placed in vials pre-treated with 2.5 g/ha of Compound 1, polymorph form A, Compound 1, polymorph form B, or Compound 1, non-crystalline amorphous oil, each from hexane anti-solvent suspension, and records the percent of B. tabaci adults above the 2 cm line over time in polymorph A vials (♦), polymorph B vials (●) or non-crystalline amorphous oil vials (▪).

FIG. 14 is a graph showing the results of a knock-down experiment in which adult white fly, B. tabaci, were placed in vials pre-treated with 0.25 g/ha of Compound 1, polymorph form A, Compound 1, polymorph form B, or Compound 1, non-crystalline amorphous oil, each from hexane anti-solvent suspension, and records the percent of B. tabaci adults above the 2 cm line over time in polymorph A vials (♦), polymorph B vials (●) or non-crystalline amorphous oil vials (▪).

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The polymorphic forms of Compound 1 and processes for preparing Compound 1 are described in detail below. In some embodiments, the amorphous form of Compound 1 can be prepared according to the methods described in are disclosed in International Patent Publication No. WO 2010/139497 A1 and U.S. Pat. No. 8,350,044, which are incorporated herein by reference in their entirety.

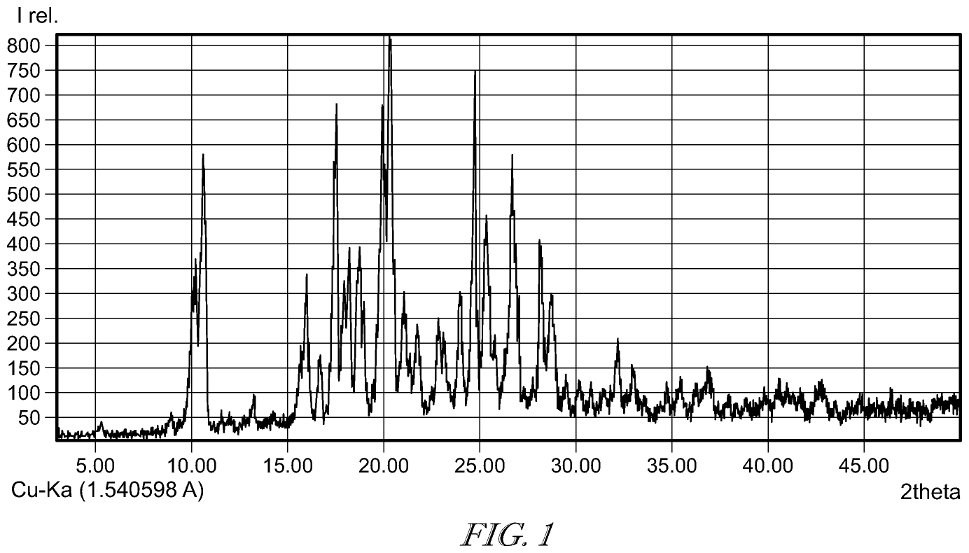

A unique physical form of N-[4-chloro-2-(pyridin-3-yl)-1,3-thiazol-5-yl]-N-ethyl-3-(methylsulfonyl) propanamide, polymorph Form A, has been prepared according to the methods described herein. The powder X-ray diffraction (PXRD) pattern of polymorph Form A is shown in FIG. 1, with corresponding tabulated data shown in Table 1.

TABLE 1

| 2theta | d-spacing | Relative Peak | Qualitative |
|---|---|---|---|
| (degrees) | (Å) | Height | intensity |
| 10.18 | 8.6823 | 348.6 | m |
| 10.62 | 8.3236 | 649.5 | s |
| 13.24 | 6.6818 | 56.2 | vw |
| 15.98 | 5.5417 | 331.4 | m |
| 16.68 | 5.3107 | 134.5 | w |
| 17.48 | 5.0694 | 686.4 | s |
| 17.92 | 4.9459 | 262 | m |
| 18.2 | 4.8704 | 378.7 | m |
| 18.7 | 4.7413 | 396.1 | m |
| 18.96 | 4.6769 | 203.9 | w |
| 19.96 | 4.4448 | 746.3 | vs |
| 20.36 | 4.3583 | 1000 | vs |
| 21.08 | 4.2111 | 246.9 | w |
| 21.74 | 4.0847 | 190.2 | w |
| 22.86 | 3.8871 | 189 | w |
| 23.14 | 3.8406 | 141.3 | w |
| 23.96 | 3.711 | 268.8 | m |
| 24.72 | 3.5986 | 765.1 | vs |
| 25.34 | 3.512 | 450 | m |
| 25.74 | 3.4583 | 99.4 | vw |
| 26.7 | 3.3361 | 598.7 | s |
| 28.16 | 3.1664 | 390.1 | m |
| 28.74 | 3.1038 | 265.7 | m |
| 32.16 | 2.7811 | 138.1 | w |
| 32.98 | 2.7138 | 97.9 | vw |

* S = strong (>50% Relative intensity), M = medium (20-50% Relative intensity), W = weak (<20% Relative intensity)

In some embodiments, the crystalline polymorph Form A of Compound 1 has a powder X-ray diffraction pattern comprising one or more peaks at diffraction angles (2θ) of 10.2±0.2, 10.6±0.2, 13.2±0.2, 16.0±0.2, 16.7±0.2, 17.5±0.2, 17.9±0.2, 18.2±0.2, 18.7±0.2, 19.0 ±0.2, 20.0±0.2, 20.4±0.2, 21.1±0.2, 21.7±0.2, 22.8±0.2, 23.1±0.2, 24.0±0.2, 24.7±0.2, 25.3±0.2, 25.7±0.2, 26.7±0.2, 28.2±0.2, 28.7±0.2, 32.2±0.2, and 33.0±0.2. In some embodiments, the crystalline polymorph Form A of Compound 1 has a powder X-ray diffraction pattern comprising one or more peaks at diffraction angles (2θ) of 10.2±0.1, 10.6±0.1, 13.2±0.1, 16.0±0.1, 16.7±0.1, 17.5±0.1, 17.9±0.1, 18.2±0.1, 18.7±0.1, 19.0±0.1, 20.0 0.1, 20.4±0.1, 21.1±0.1, 21.7±0.1, 22.8±0.1, 23.1±0.1, 24.0=0.1, 24.7±0.1, 25.3 ±0.1, 25.7±0.1, 26.7±0.1, 28.2±0.1, 28.7±0.1, 32.2±0.1, and 33.0±0.1. In some embodiments, the crystalline polymorph Form A of Compound 1 has a powder X-ray diffraction pattern comprising a combination of two or more peaks at diffraction angles (2θ) as provided in the above embodiments. It will be appreciated that the diffraction angles (2θ) provided in Table 1 are within the experimental error of the values provided above and also referred to in the present disclosure.

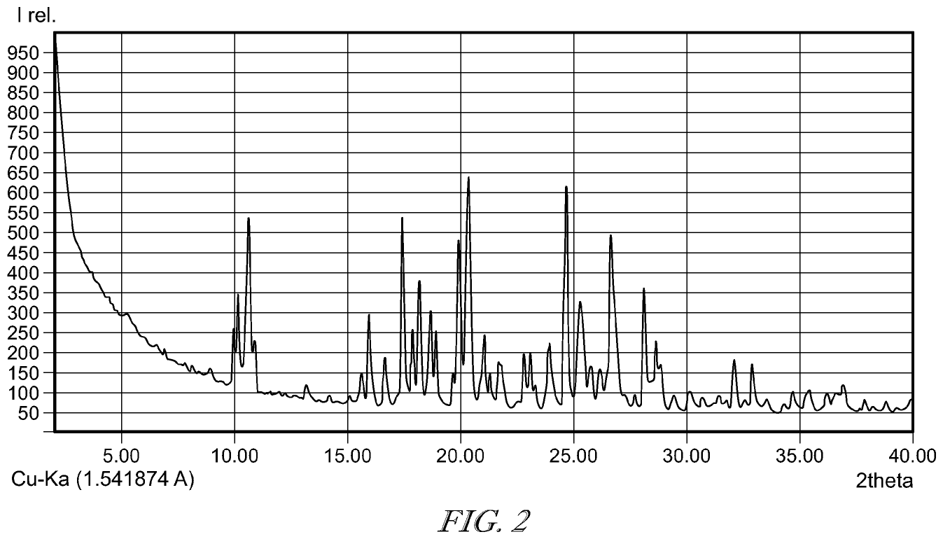

In some embodiments, the same physical form of N-[4-chloro-2-(pyridin-3-yl)-1,3-thiazol-5-yl]-N-ethyl-3-(methylsulfonyl) propanamide, as shown in FIG. 1 and in Table 1, polymorph Form A, has been prepared according to the methods described herein, wherein the material can be prepared with higher purity using recrystallization techniques, as described herein, such as the use of seed crystals of polymorph Form A, multiple crystallization/recrystallization methods, and the like. It will be appreciated by one of skill in the art that the use of such techniques to provide crystalline material of higher purity can provide higher resolution in powder X-ray diffraction (PXRD) analysis. The powder X-ray diffraction (PXRD) pattern of polymorph Form A using such techniques to obtain higher purity material, and thus higher quality powder X-ray diffraction (PXRD) analysis, as shown in FIG. 2, with corresponding tabulated data shown in Table 2. It will be appreciated by one of skill in the art that the PXRD data shown in Table 2 are within experimental error of the data provided in Table 1, and that the higher resolution PXRD pattern shown in FIG. 2 provided enhanced ability to identify closely spaced peaks in the PXRD pattern that were not previously visible in the PXRD pattern shown in FIG. 1.

TABLE 2

| 2theta | d-spacing | Relative Peak | Qualitative |
|---|---|---|---|
| (degrees) | (Å) | Height | intensity |
| 9.94 | 8.8988 | 260.9 | m |
| 10.12 | 8.7337 | 353.9 | m |
| 10.6 | 8.3392 | 726.5 | s |
| 10.86 | 8.1469 | 228.2 | w |
| 13.15 | 6.7329 | 121 | w |
| 15.6 | 5.6758 | 121.1 | w |
| 15.94 | 5.5555 | 365.7 | m |
| 16.62 | 5.3297 | 191.1 | w |
| 17.42 | 5.0867 | 777.7 | s |
| 17.86 | 4.9665 | 256.2 | m |
| 18.16 | 4.8811 | 529.1 | s |
| 18.66 | 4.7514 | 393.9 | m |
| 18.9 | 4.6916 | 279.3 | m |
| 19.9 | 4.458 | 739 | m |
| 20.32 | 4.3668 | 1000 | vs |
| 21.04 | 4.219 | 309.3 | m |
| 21.3 | 4.1715 | 149 | w |
| 21.68 | 4.0959 | 195.3 | w |
| 21.78 | 4.0807 | 169.7 | w |
| 22.8 | 3.8971 | 223.9 | w |
| 23.08 | 3.8505 | 219.7 | w |
| 23.28 | 3.821 | 118.6 | w |
| 23.92 | 3.7171 | 277.1 | m |
| 24.66 | 3.6072 | 943.3 | vs |
| 25.28 | 3.5202 | 478 | m |
| 25.72 | 3.4609 | 175.6 | w |
| 26.16 | 3.4037 | 165.8 | w |
| 26.64 | 3.3435 | 749.4 | vs |
| 28.1 | 3.173 | 516.8 | s |
| 28.64 | 3.1144 | 294.4 | m |
| 28.84 | 3.0932 | 147.3 | w |
| 32.1 | 2.7861 | 226.4 | w |
| 32.9 | 2.7202 | 201.6 | w |

* S = strong (>50% Relative intensity), M = medium (20-50% Relative intensity), W = weak (<20% Relative intensity)

In some embodiments, the crystalline polymorph Form A of Compound 1 has a powder X-ray diffraction pattern comprising one or more peaks at diffraction angles (2θ) of 10.0±0.2, 10.1±0.2, 10.6±0.2, 10.9±0.2, 13.2±0.2, 15.6±0.2, 16.0±0.2, 16.6±0.2, 17.4±0.2, 17.9 ±0.2, 18.2±0.2, 18.7±0.2, 18.9±0.2, 19.9±0.2, 20.3±0.2, 21.0±0.2, 21.3±0.2, 21.7±0.2, 21.8±0.2, 22.8±0.2, 23.1±0.2, 23.3±0.2, 23.9±0.2, 24.7±0.2, 25.3±0.2, 25.7±0.2, 26.2±0.2, 26.6±0.2, 28.1±0.2, 28.6±0.2, 28.8±0.2, 32.1±0.2, and 32.9±0.2. In some embodiments, the crystalline polymorph Form A of Compound 1 has a powder X-ray diffraction pattern comprising one or more peaks at diffraction angles (2θ) of 10.0±0.1, 10.1±0.1, 10.6±0.1, 10.9±0.1,13.2±0.1,15.6±0.1,16.0±0.1,16.6±0.1,17.4±0.1,17.9±0.1, 18.2±0.1, 18.7±0.1, 18.9±0.1, 19.9±0.1, 20.3±0.1, 21.0±0.1, 21.3±0.1, 21.7±0.1, 21.8 ±0.1, 22.8±0.1, 23.1±0.1, 23.3±0.1, 23.9±0.1, 24.7±0.1, 25.3±0.1, 25.7±0.1, 26.2±0.1, 26.6±0.1, 28.1±0.1, 28.6±0.1, 28.8±0.1, 32.1±0.1, and 32.9±0.1. In some embodiments, the crystalline polymorph Form A of Compound 1 has a powder X-ray diffraction pattern comprising a combination of two or more peaks at diffraction angles (2θ) as provided in the above embodiments. It will be appreciated that the diffraction angles (2θ) provided in Table 2 are within the experimental error of the values provided above and also referred to in the present disclosure.

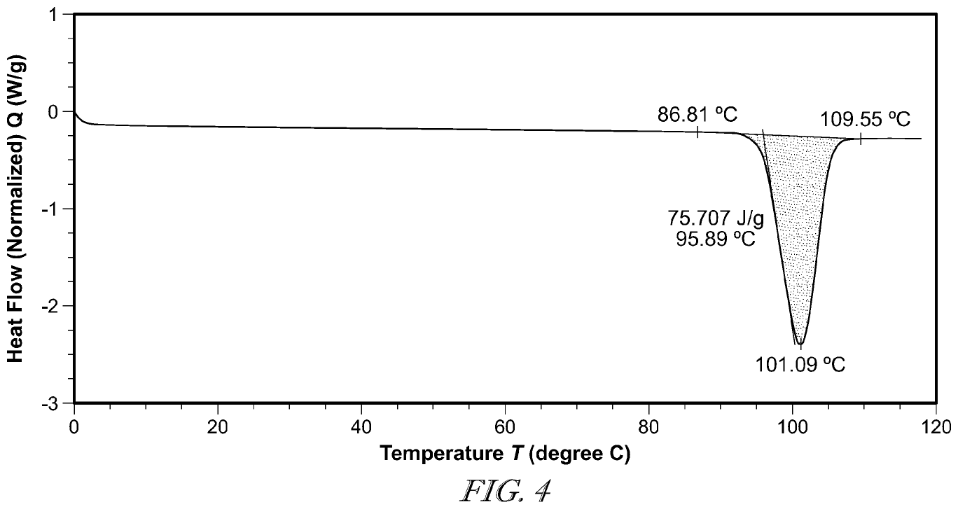

The DSC thermogram for crystalline polymorph form A is shown in FIG. 4. It has been determined that during the DSC method as described in Example 10, polymorph form A melted at about 101.09° C. and a heat of Fusion=75.707 J/g, as shown in FIG. 4.

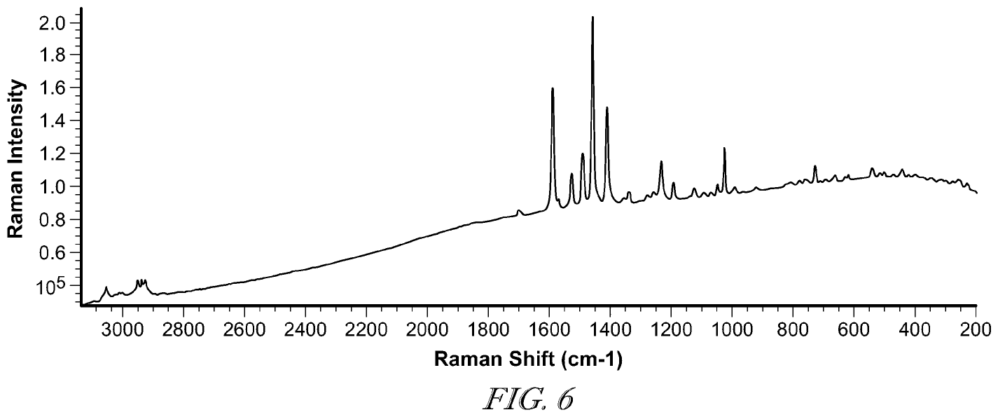

The Raman spectrum for crystalline polymorph form A is shown in FIG. 6.

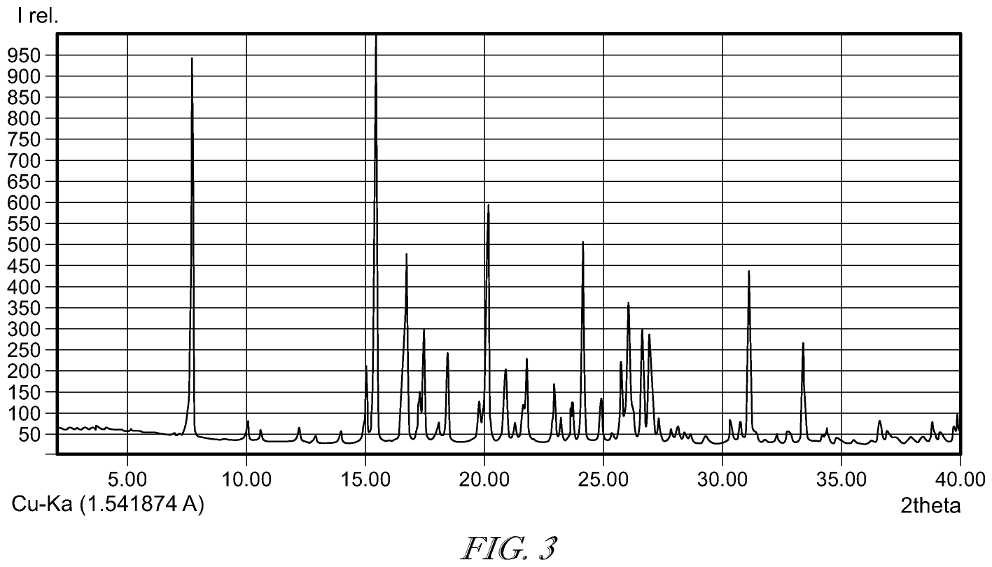

A unique physical form of N-[4-chloro-2-(pyridin-3-yl)-1,3-thiazol-5-yl]-N-ethyl-3-(methylsulfonyl) propanamide (solvent-free and anhydrous), polymorph Form B, has been prepared according to the methods described herein. The powder X-ray diffraction (PXRD) pattern of polymorph Form B is shown in FIG. 3, with corresponding tabulated data shown in Table 3.

TABLE 3

| 2theta | d-spacing | Relative Peak | Qualitative |
|---|---|---|---|
| (degrees) | (Å) | Height | intensity |
| 7.72 | 11.4426 | 901.4 | vs |
| 10.06 | 8.7856 | 47.3 | vw |
| 10.63 | 8.3226 | 61.8 | vw |
| 12.2 | 7.2489 | 32.1 | vw |
| 12.93 | 6.8469 | 44 | vw |
| 13.96 | 6.3387 | 27.4 | vw |
| 15.04 | 5.8859 | 193.7 | w |
| 15.44 | 5.7343 | 1000 | vs |
| 16.58 | 5.3469 | 212.5 | w |
| 16.74 | 5.2918 | 472.9 | m |
| 17.27 | 5.1348 | 151.1 | w |
| 17.46 | 5.0752 | 280.7 | m |
| 18.08 | 4.9025 | 49.7 | vw |
| 18.44 | 4.8076 | 223.6 | w |
| 19.79 | 4.4863 | 127.6 | w |
| 20.16 | 4.4011 | 619.2 | s |
| 20.88 | 4.251 | 185.2 | w |
| 21.3 | 4.1681 | 47.9 | vw |
| 21.61 | 4.1124 | 119.3 | w |
| 21.78 | 4.0773 | 215.4 | w |
| 22.92 | 3.877 | 151.3 | w |
| 23.22 | 3.8276 | 55.1 | vw |
| 23.68 | 3.7543 | 103.2 | w |
| 24.14 | 3.6838 | 508.2 | s |
| 24.9 | 3.573 | 110.8 | w |
| 25.38 | 3.5065 | 23.3 | vw |
| 25.74 | 3.4583 | 199.5 | w |
| 26.06 | 3.4165 | 361.3 | m |
| 26.64 | 3.3435 | 278.8 | m |
| 26.94 | 3.3069 | 273.4 | m |
| 27.34 | 3.2594 | 57.8 | vw |
| 27.84 | 3.202 | 35.6 | vw |
| 28.12 | 3.1708 | 45.6 | vw |
| 28.42 | 3.138 | 30 | vw |
| 28.66 | 3.1122 | 22.2 | vw |
| 29.32 | 3.0437 | 21.7 | vw |
| 30.34 | 2.9436 | 59.6 | vw |
| 30.74 | 2.9062 | 54.2 | vw |
| 31.12 | 2.8716 | 412.7 | m |
| 32.28 | 2.7733 | 48.9 | vw |
| 32.8 | 2.7305 | 52.9 | vw |
| 33.4 | 2.6806 | 243.6 | w |
| 34.2 | 2.6219 | 48.4 | vw |
| 34.4 | 2.6049 | 33.2 | vw |
| 34.82 | 2.5766 | 42.5 | vw |
| 35.54 | 2.526 | 37.5 | vw |
| 36.6 | 2.4532 | 56 | vw |
| 36.92 | 2.4327 | 28.5 | vw |
| 38.44 | 2.3399 | 17.9 | vw |
| 38.82 | 2.3179 | 48.2 | vw |
| 39.88 | 2.2587 | 35.7 | vw |

* S = strong (>50% Relative intensity), M = medium (20-50% Relative intensity), W = weak (<20% Relative intensity)

In some embodiments, the crystalline polymorph Form B of Compound 1 has a powder X-ray diffraction pattern comprising one or more peaks at diffraction angles (2θ) of 7.7±0.2, 10.1±0.2, 10.6±0.2, 12.2±0.2, 12.9±0.2, 14.0±0.2, 15.0±0.2, 15.4±0.2, 16.6±0.2, 16.7 ±0.2, 17.3±0.2, 17.5±0.2, 18.1±0.2, 18.5±0.2, 19.8±0.2, 20.2±0.2, 20.9±0.2, 21.3±0.2, 21.6±0.2, 21.8±0.2, 22.9±0.2, 23.2±0.2, 23.7±0.2, 24.1±0.2, 24.9±0.2, 25.4±0.2, 25.8±0.2, 26.1±0.2, 26.6±0.2, 27.0±0.2, 27.3±0.2, 27.8±0.2, 28.1±0.2, 28.4±0.2, 28.7 ±0.2, 29.3±0.2, 30.3±0.2, 30.7±0.2, 31.1±0.2, 32.3±0.2, 32.8±0.2, 33.4±0.2, 34.2±0.2, 34.4±0.2, 34.8±0.2, 35.5±0.2, 36.6±0.2, 36.9±0.2, 38.4±0.2, 38.8±0.2, and 39.9±0.2. In some embodiments, the crystalline polymorph Form B of Compound 1 has a powder X-ray diffraction pattern comprising one or more peaks at diffraction angles (2θ) of 7.7±0.1, 10.1 ±0.1, 10.6±0.1, 12.2±0.1, 12.9±0.1, 14.0±0.1, 15.0±0.1, 15.4±0.1, 16.6±0.1, 16.7±0.1, 17.3±0.1, 17.5±0.1, 18.1±0.1, 18.5±0.1, 19.8±0.1,20.2±0.1,20.9±0.1,21.3±0.1, 21.6±0.1, 21.8±0.1, 22.9±0.1, 23.2±0.1, 23.7±0.1, 24.1±0.1, 24.9±0.1, 25.4±0.1, 25.8 ±0.1, 26.1±0.1, 26.6±0.1, 27.0±0.1, 27.3±0.1, 27.8±0.1, 28.1±0.1, 28.4±0.1, 28.7±0.1, 29.3±0.1, 30.3±0.1,30.7±0.1,31.1±0.1,32.3±0.1,32.8±0.1,33.4±0.1,34.2±0.1, 34.4±0.1, 34.8±0.1, 35.5±0.1, 36.6±0.1, 36.9±0.1, 38.4±0.1, 38.8±0.1, and 39.9±0.1. In some embodiments, the crystalline polymorph Form B of Compound 1 has a powder X-ray diffraction pattern comprising a combination of two or more peaks at diffraction angles (2θ) as provided in the above embodiments. It will be appreciated that the diffraction angles (2θ) provided in Table 3 are within the experimental error of the values provided above and also referred to in the present disclosure.

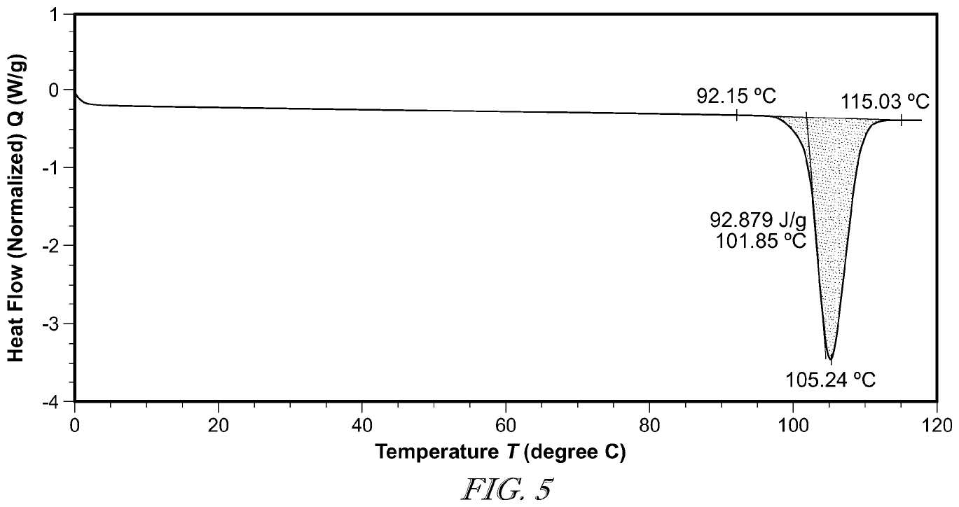

The DSC thermogram for crystalline polymorph form A is shown in FIG. 5. It has been determined that during the DSC method as described in Example 10, polymorph form A melted at about 105.24° C. and a heat of Fusion=92.879 J/g, as shown in FIG. 5.

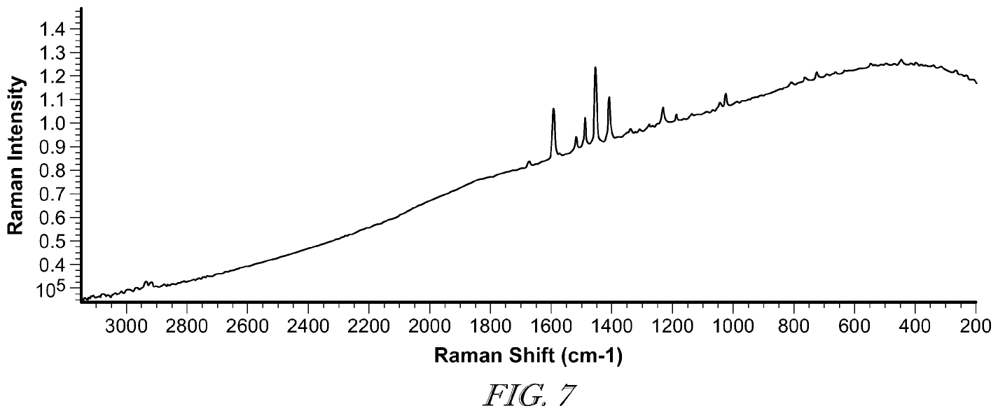

The Raman spectrum for crystalline polymorph form A is shown in FIG. 7.

### Combinations

In some embodiments, any one of polymorph Forms A and B of Compound 1 may be used in combination (such as, in a compositional mixture, or a simultaneous or sequential application) with one or more active ingredients.

In some embodiments, any one or more of polymorph Forms A and B of Compound 1 may be used in combination (such as, in a compositional mixture, or a simultaneous or sequential application) with one or more active ingredients each having an insecticidal mode of action (MoA) that is the same as, similar to, but more likely different from, the MoA of any one or more of polymorph Forms A and B of Compound 1. In some embodiments, any one or more of polymorph Forms A and B of Compound 1 may be used in combination (such as, in a compositional mixture, or a simultaneous or sequential application) with one or more molecules having acaricidal, algicidal, avicidal, bactericidal, fungicidal, herbicidal, insecticidal, molluscicidal, nematicidal, rodenticidal, and/or virucidal properties.

In some embodiments, any one or more of polymorph Forms A and B of Compound 1 may be used in combination (such as, in a compositional mixture, or a simultaneous or sequential application) with one or more molecules that are antifeedants, bird repellents, chemosterilants, herbicide safeners, insect attractants, insect repellents, mammal repellents, mating disrupters, plant activators, plant growth regulators, and/or synergists.

In some embodiments, any one or more of polymorph Forms A and B of Compound 1 may also be used in combination (such as in a compositional mixture, or a simultaneous or sequential application) with one or more biopesticides.

In some embodiments, in a pesticidal composition combination of any one or more of polymorph Forms A and B of Compound 1 and an active ingredient may be used in a wide variety of weight ratios. For example, in a two-component mixture, the weight ratio of any one or more of polymorph Forms A and B of Compound 1 to an active ingredient, various ratios may be used. However, in general, weight ratios less than about 10:1 to about 1:10 are preferred. It is also preferred sometimes to use a three, four, five, six, seven, or more, component mixture comprising any one or more of polymorph Forms A and B of Compound 1 and an additional two or more active ingredients.

Weight ratios of any one or more of polymorph Forms A and B of Compound 1 to an active ingredient may also be depicted as X: Y; wherein X is the parts by weight of any one or more of polymorph Forms A and B of Compound 1 and Y is the parts by weight of active ingredient. The numerical range of the parts by weight for X is 0<X≤100 and the parts by weight for Y is 0<Y<100. By way of non-limiting example, the weight ratio of any one or more of polymorph Forms A and B of Compound 1 to an active ingredient may be 20:1.

### Formulations

A pesticide is many times not suitable for application in its pure form. It is usually necessary to add other substances so that the pesticide may be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water-soluble liquids, water-dispersible granules or dry flowables, wettable powders, and ultra-low volume solutions.

Pesticides are applied most often as aqueous suspensions or emulsions prepared from concentrated formulations of such pesticides. Such water-soluble, water-suspendable, or emulsifiable formulations are either solids, usually known as wettable powders, water-dispersible granules, liquids usually known as emulsifiable concentrates, or aqueous suspensions. Wettable powders, which may be compacted to form water-dispersible granules, comprise an intimate mixture of the pesticide, a carrier, and surfactants. The concentration of the pesticide is usually from about 10% to about 90% by weight. The carrier is usually selected from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates of pesticides comprise a convenient concentration of a pesticide, such as from about 50 to about 500 grams per liter (g/L) of liquid dissolved in a carrier that is either a water-miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums, may also be added to increase the density and viscosity of the aqueous carrier. It is often most effective to grind and mix the pesticide at the same time by preparing the aqueous mixture and homogenizing it in an implement such as a sand mill, ball mill, or piston-type homogenizer. The pesticide in suspension might be microencapsulated in plastic polymer.

Oil dispersions (OD) comprise suspensions of organic solvent-insoluble pesticides finely dispersed in a mixture of organic solvent and emulsifiers at a concentration in the range from about 2% to about 50% by weight. One or more pesticide might be dissolved in the organic solvent. Useful organic solvents include aromatics, especially xylenes and petroleum fractions, especially the high-boiling naphthalenic and olefinic portions of petroleum such as heavy aromatic naphtha. Other solvents may include vegetable oils, seed oils, and esters of vegetable and seed oils. Suitable emulsifiers for oil dispersions are selected from conventional anionic and non-ionic surfactants. Thickeners or gelling agents are added in the formulation of oil dispersions to modify the rheology or flow properties of the liquid and to prevent separation and settling of the dispersed particles or droplets.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier, which has been pre-formed to the appropriate particle size, in the range of from about 0.5 millimeters (mm) to about 3 mm. Such compositions may also be formulated by making a dough or paste of the carrier and molecule, and then crushing and drying to obtain the desired granular particle size. Another form of granules is a water-emulsifiable granule (EG). It is a formulation consisting of granules to be applied as a conventional oil-in-water emulsion of the active ingredient(s), either solubilized or diluted in an organic solvent, after disintegration and dissolution in water. Water-emulsifiable granules comprise one or several active ingredient(s), either solubilized or diluted in a suitable organic solvent that is (are) absorbed in a water soluble polymeric shell or some other type of soluble or insoluble matrix.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. Dusts may be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions, the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait, they also consume the pesticide. Baits may take the form of granules, gels, flowable powders, liquids, or solids. Baits may be used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings, or in special chambers.

Pesticides may be microencapsulated by suspending the pesticide particles or droplets in plastic polymers of various types. By altering, the chemistry of the polymer or by changing factors in the processing, microcapsules may be formed of various sizes, solubility, wall thicknesses, and degrees of penetrability. These factors govern the speed with which the active ingredient within is released, which in turn, affects the residual performance, speed of action, and odor of the product. The microcapsules might be formulated as suspension concentrates or water dispersible granules.

Oil solution concentrates are made by dissolving pesticide in a solvent that will hold the pesticide in solution, oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one molecule which is agriculturally active, and is individually coated with a monolamellar or oligolamellar layer comprising: (1) at least one non-ionic lipophilic surface-active agent, (2) at least one non-ionic hydrophiilc surface-active agent, and (3) at least one ionic surface-active agent, wherein the globules having a mean particle diameter of less than 800 nanometers.

### Other Formulation Components

Generally, when any one or more of polymorph Forms A and B of Compound 1 is used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate, sodium dioctyl sulfosuccinate, alkyl phenol ethoxylates, and aliphatic alcohol ethoxylates.

A dispersing agent is a substance that adsorbs onto the surface of particles, helps to preserve the state of dispersion of the particles, and prevents them from reaggregating. Dispersing agents are added to agrochemical formulations to facilitate dispersion and suspension during manufacture, and to ensure the particles redisperse into water in a spray tank. They are widely used in wettable powders, suspension concentrates, and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium-naphthalene-sulfonate-formaldehyde-condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces.

Examples of dispersing agents used in agrochemical formulations are: sodium lignosulfonates, sodium naphthalene sulfonate formaldehyde condensates, tristritylphenol-ethoxylate-phosphate-esters, aliphatic alcohol ethoxylates, alkyl ethoxylates, EO-PO block copolymers, and graft copolymers.

An emulsifying agent is a substance that stabilizes a suspension of droplets of one liquid phase in another liquid phase. Without the emulsifying agent, the two liquids would separate into two immiscible liquid phases. The most commonly used emulsifier blends contain an alkylphenol or an aliphatic alcohol with twelve or more ethylene oxide units and the oil-soluble calcium salt of dodecyl benzenesulfonic acid. A range of hydrophile-lipophite balance (“HLB”) values from about 8 to about 18 will normally provide good stable emulsions. Emulsion stability can sometimes be improved by the addition of a small amount of an EO-PO block copolymer surfactant.

A solubilizing agent is a surfactant that will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates, linear aliphatic alcohol ethoxylates, and aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, oil dispersions, and ultra-low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C₉ and C₁₀ aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, oil dispersions, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate and oil dispersion formulations using clays and silicas. Examples of these types of materials, include, but are not limited to, montmorillonite, bentonite, magnesium aluminum silicate, and attapulgite. Water-soluble polysaccharides in water-based suspension concentrates have been used as thickening-gelling agents for many years, the types of polysaccharides most commonly used are natural extracts of seeds and seaweeds or are synthetic derivatives of cellulose. Examples of these types of materials include, but are not limited to, guar gum, locust bean gum, carrageenan, alginates, methyl cellulose, sodium carboxymethyl cellulose (SCMC), and hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore, preservation agents are used to eliminate or reduce their effect. Examples of such agents include, but are not limited to: propionic acid and its sodium salt, sorbic acid and its sodium or potassium salts, benzoic acid and its sodium salt, p-hydroxybenzoic acid sodium salt, methyl p-hydroxybenzoate, and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g. plant and animal sources. Specific examples are: vegetable oils, seed oils, and esters thereof.

### Applications

Any one or more of polymorph Forms A and B of Compound 1 may be applied to any locus. Particular loci to apply such molecules include loci where alfalfa, almonds, apples, barley, beans, canola, corn, cotton, crucifers, flowers, fodder species (Rye Grass, Sudan Grass, Tall Fescue, Kentucky Blue Grass, and Clover), fruits, lettuce, oats, oil seed crops, oranges, peanuts, pears, peppers, potatoes, rice, sorghum, soybeans, strawberries, sugarcane, sugarbeets, sunflowers, tobacco, tomatoes, wheat (for example, Hard Red Winter Wheat, Soft Red Winter Wheat, White Winter Wheat, Hard Red Spring Wheat, and Durum Spring Wheat), and other valuable crops are growing or the seeds thereof are going to be planted.

Any one or more of polymorph Forms A and B of Compound 1 may also be applied where plants, such as crops, are growing and where there are low levels (even no actual presence) of pests that can commercially damage such plants. Applying such molecules in such locus is to benefit the plants being grown in such locus. Such benefits, may include, but are not limited to: helping the plant grow a better root system; helping the plant better withstand stressful growing conditions; improving the health of a plant; improving the yield of a plant (e.g. increased biomass and/or increased content of valuable ingredients); improving the vigor of a plant (e.g. improved plant growth and/or greener leaves); improving the quality of a plant (e.g. improved content or composition of certain ingredients); and improving the tolerance to abiotic and/or biotic stress of the plant.

Any one or more of polymorph Forms A and B of Compound 1 may be applied with ammonium sulfate when growing various plants as this may provide additional benefits.

Any one or more of polymorph Forms A and B of Compound 1 may be applied on, in, or around plants genetically modified to express specialized traits, such as Bacillus thuringiensis (for example, Cry1Ab, Cry1Ac, Cry1Fa, Cry1A.105, Cry2Ab, Vip3A, mCry3A, Cry3Ab, Cry3Bb, Cry34Ab1/Cry35Ab1), other insecticidal toxins, or those expressing herbicide tolerance, or those with “stacked” foreign genes expressing insecticidal toxins, herbicide tolerance, nutrition-enhancement, or any other beneficial traits.

Any one or more of polymorph Forms A and B of Compound 1 may be applied to the foliar and/or fruiting portions of plants to control pests. Either such polymorph of Compound 1 will come in direct contact with the pest, or the pest will consume such polymorph of Compound 1 when eating the plant or while extracting sap or other nutrients from the plant.

Any one or more of polymorph Forms A and B of Compound 1 may also be applied to the soil, and when applied in this manner, root and stem feeding pests may be controlled. The roots may absorb such molecules thereby taking it up into the foliar portions of the plant to control above ground chewing and sap feeding pests.

Systemic movement of pesticides in plants may be utilized to control pests on one portion of the plant by applying (for example by spraying a locus) a Any one or more of polymorph Forms A and B of Compound 1 to a different portion of the plant. For example, control of foliar-feeding insects may be achieved by drip irrigation or furrow application, by treating the soil with for example pre-or post-planting soil drench, or by treating the seeds of a plant before planting.

Any one or more of polymorph Forms A and B of Compound 1 may be used with baits. Generally, with baits, the baits are placed in the ground where, for example, termites can come into contact with, and/or be attracted to the bait. Baits can also be applied to a surface of a building, (horizontal, vertical, or slant surface) where, for example, ants, termites, cockroaches, and flies can come into contact with, and/or be attracted to, the bait.

Any one or more of polymorph Forms A and B of Compound 1 may be encapsulated inside, or placed on the surface of a capsule. The size of the capsules can range from nanometer size (about 100-900 nanometers in diameter) to micrometer size (about 10-900 microns in diameter).

Any one or more of polymorph Forms A and B of Compound 1 may be applied to eggs of pests. Because of the unique ability of the eggs of some pests to resist certain pesticides, repeated applications of such molecules may be desirable to control newly emerged larvae.

Any one or more of polymorph Forms A and B of Compound 1 may be applied as seed treatments. Seed treatments may be applied to all types of seeds, including those from which plants genetically modified to express specialized traits will germinate. Representative examples include those expressing proteins toxic to invertebrate pests, such as Bacillus thuringiensis or other insecticidal toxins, those expressing herbicide tolerance, such as “Roundup Ready” seed, or those with “stacked” foreign genes expressing insecticidal toxins, herbicide tolerance, nutrition-enhancement, drought tolerance, or any other beneficial traits, Furthermore, such seed treatments with any one or more of polymorph Forms A and B of Compound 1 may further enhance the ability of a plant to withstand stressful growing conditions better. This results in a healthier, more vigorous plant, which can lead to higher yields at harvest time. Generally, amounts of about 1 gram of such polymorph to about 500 grams per 100,000 seeds are expected to provide good benefits; amounts from about 10 grams to about 100 grams per 100,000 seeds are expected to provide better benefits; and amounts from about 25 grams to about 75 grams per 100,000 seeds are expected to provide even better benefits. Any one or more of polymorph Forms A and B of Compound 1 may be applied with one or more active ingredients in a soil amendment.

Any one or more of polymorph Forms A and B of Compound 1 may be used for controlling endoparasites and ectoparasites in the veterinary medicine sector or in the field of non-human-animal keeping. Such molecules may be applied by oral administration in the form of, for example, tablets, capsules, drinks, and granules; by dermal application in the form of, for example, dipping, spraying, pouring on, spotting on, and dusting; and by parenteral administration in the form of, for example, an injection.

Any one or more of polymorph Forms A and B of Compound 1 may also be employed advantageously in livestock keeping, for example, cattle, chickens, geese, goats, pigs, sheep, and turkeys. They may also be employed advantageously in pets such as, horses, dogs, and cats. Particular pests to control would be flies, fleas, and ticks that are bothersome to such animals. Suitable formulations are administered orally to the animals with the drinking water or feed. The dosages and formulations that are suitable depend on the species.

Any one or more of polymorph Forms A and B of Compound 1 may also be used for controlling parasitic worms, especially of the intestine, in the animals listed above. Any one or more of polymorph Forms A and B of Compound 1 may also be employed in therapeutic methods for non-human health care, such methods include, but are not limited to, oral administration in the form of, for example, tablets, capsules, drinks, and granules, and by dermal application.

Polymorph Forms A and B of Compound 1 may also be applied to invasive pests. Pests around the world have been migrating to new environments (for such pest) and thereafter becoming a new invasive species in such new environment. Such polymorphs may also be used on such new invasive species to control them in such new environments.

### EXAMPLES

The examples and preparations provided below further illustrate and exemplify particular aspects of embodiments of the disclosure. It is to be understood that the scope of the present disclosure is not limited in any way by the scope of the following examples.

### Example 1

### Synthesis of N-[4-chloro-2-(pyridin-3-yl)-1,3-thiazol-5-yl]-N-ethyl-3-(methylsulfonyl) propanamide (Compound 1), and Isolation as Form A

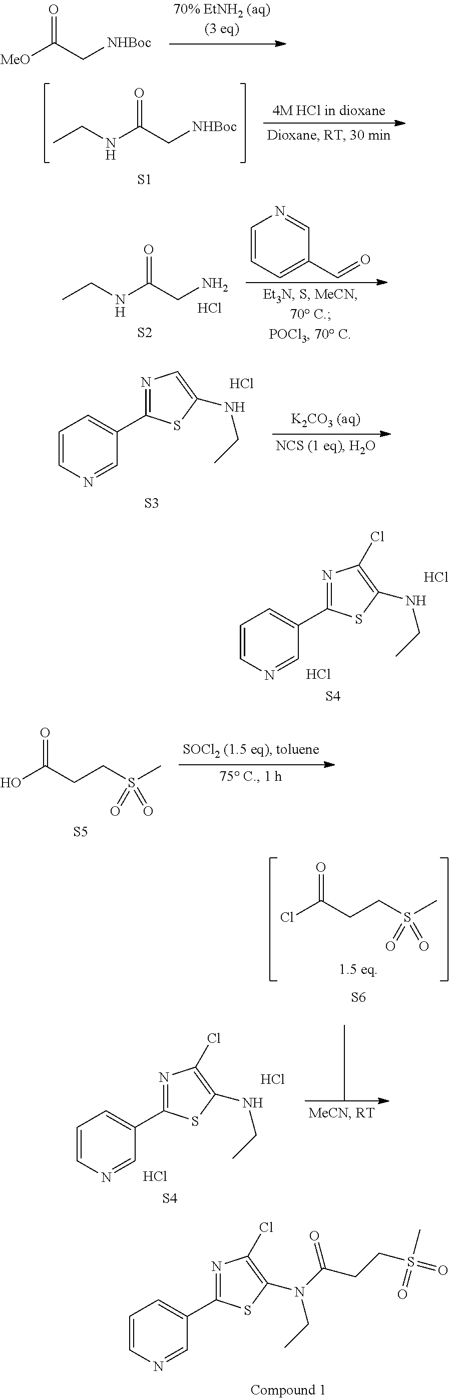

### Step 1: Preparation of tert-butyl (2-(ethylamino)-2-oxoethyl) carbamate (S1)

To a 1 L round bottomed was added 70% ethylamine in water (347 mL, 4360 mmol, 3 equiv) and methyl (tert-butoxycarbonyl) glycinate (255 ml, 1453 mmol) was added in portions to keep the temperature <35° C. The reaction was stirred at room temperature and monitored by NMR using CDCl₃ The excess ethylamine and water were removed by atmospheric distillation The mixture was cooled and cyclopentyl methyl ether (CPME) (400 mL) was added. Atmospheric distillation was continued to azeotrope out the remaining water (pot temperature: 115° C., overhead temperature: 95° C.). ˜260 mL of CPME was distilled overhead. The mixture was cooled to room temperature and CPME (140 mL) was added to the bottoms. This solution was used in the next step without further manipulation. ¹H NMR (400 MHz, Chloroform-d) δ 3.77 (d, J=5.9 Hz, 2H), 3.37-3.26 (m, 2H), 1.46 (s, 9H), 1.15 (t, J=7.3 Hz, 3H).

### Step 2: Step 2: Preparation of 2-amino-N-ethylacetamide hydrochloride (S2)

To a 5 L jacketed reactor was added 6N HCl in isopropanol (1055 mL, 6.3 mol, 4 equiv) and CPME (1055 mL) and the jacket was set at 30° C. tert-butyl (2-(ethylamino)-2-oxoethyl) carbamate (assumed 320 g, 1582 mmol) as a solution in CPME from the previous reaction was added via peristaltic pump over 30 minutes. The reaction was stirred at 30° C. for 6 h and cooled to 25° C. for stirring overnight. The reaction was monitored by NMR in DMSO. The reaction formed a very thick slurry. The slurry was diluted with CPME (650 mL) and the solids were isolated by filtration through a coarse glass frit, washing with CPME (300 mL). The wet cake was then washed with one cake volume of hexanes to help remove CPME for drying. The material was dried in a vacuum oven at 45° C. giving 2-amino-N-ethylacetamide hydrochloride (S2) (184.11 g, 1315 mmol, 83% yield) as a fluffy white solid. ¹H NMR (400 MHz, DMSO-d₆) δ 3.49 (s, 2H), 3.14 (qd, J=7.3, 5.4 Hz, 2H), 1.05 (t, J=7.3 Hz, 3H).

### Step 3: Preparation of N-ethyl-2-(pyridin-3-yl)-1,3-thiazol-5-amine dihydrochloride (S3)

To a 125 mL 3 neck straight wall flat bottom flask equipped with reflux condenser and thermocouple was added 2-amino-N-ethylacetamide hydrochloride (4.99 g, 36.0 mmol, 1.25 equiv.), anhydrous acetonitrile (48 mL) leading to a white slurry. The reactor was inerted with nitrogen. Anhydrous triethylamine (99%, 5.60 mL, 39.7 mol, 1.38 equiv.) was added and the mixture was stirred for 1 h leading to a white thick slurry. Nicotinaldehyde (98%, 2.70 mL, 28.8 mmol, 1.0 equiv.) was added leading to a thinner slurry. Sulfur solid powder (1.20 g, 37.4 mmol, 1.30 equiv.) was added. The mixture was stirred at 70° C. and gradually became a dark red orange solution. The reaction was monitored by HPLC for disappearance of nicotinaldehyde which took ˜5 h and then cooled to 50° C. Phosphorous oxychloride (POCl3, 99%, 6.70 mL, 77.8 mmol, 2.50 equiv.) was added dropwise to the reaction mixture while keeping the pot temperature bellow 60° C. The dark brown thin slurry/oil was stirred at 50° C. for 7 h over which time a yellow slurry formed (monitored by HPLC). The yellow orange slurry was cooled to 15° C. and toluene (20 mL) was added. The mixture was filtered and washed with toluene (3×10 mL). The yellow wet cake was dried under vacuum at 40° C. for 16 h to afford N-ethyl-2-(pyridin-3-yl)-1.3-thiazol-5-amine dihydrochloride (S3) as yellow solid (5.14 g) with a purity of 92 wt % as measured by 1H NMR assay indicating 57.8% yield over the two steps. ¹H NMR (400 MHz, DMSO-d₆) δ 9.07 (d, J=2.1 Hz, 1H), 8.71 (dd, J=5.6, 1.3 Hz, 1H), 8.66 (ddd, J=8.3, 2.2, 1.3 Hz, 1H), 7.97 (ddd, J=8.3, 5.5, 0.7 Hz, 1H), 7.54-7.19 (m, 1H), 7.02 (s, 1H), 3.15 (q, J=7.2 Hz, 2H), 1.21 (t, J=7.2 Hz, 3H). ¹³C NMR (101 MHz, DMSO) δ 155.15, 140.81, 139.39, 139.16, 136.62, 133.13, 127.52, 120.35, 41.56, 13.89. ESIMS m/z 206 [(M+H−2HCl)+].

### Step 4: Step 4: Preparation of 4-chloro-N-ethyl-2-(pyridin-3-yl) thiazol-5-amine dihydrochloride (S4)

To a 1 L round bottomed flask was added N-ethyl-2-(pyridin-3-yl)-1,3-thiazol-5-amine dihydrochloride (30 g, 108 mmol) and water (360 mL) and the resulting orange/red solution was cooled to ˜5° C. with an ice bath. N-chlorosuccinimide (14.4 g, 108 mmol, 1 equiv.) was added in portions keeping the temperature below 7° C. The resulting dark solution was stirred at 5° C. for 40 min monitoring by HPLC. The reaction was poured into 20% potassium carbonate solution causing a gummy red solid to form and the product was extracted with ethyl acetate. The organic layer was washed with 10% sodium thiosulfate followed by 20% potassium carbonate. The organic layer was dried over sodium sulfate, filtered, and concentrated giving 4-chloro-N-ethyl-2-(pyridin-3-yl)-1,3-thiazol-5-amine (S4) (27 g, 104%) as a red oil that was carried into Step 6 without further purification. ¹H NMR (300 MHz, CDCl₃) δ 8.96 (dd, J=2.4, 0.8 Hz, 1H), 8.54 (dd, J=4.8, 1.6 Hz, 1H), 8.07 (ddd, J=8.1, 2.4, 1.6 Hz, 1H), 7.31 (ddd, J=8.1, 4.8, 0.9 Hz, 1H), 4.02 (s, 1H), 3.27 (qd, J=7.2, 5.8 Hz, 2H), 1.34 (t, J=7.2 Hz, 3H).

### Step 5: Preparation of 3-(methylsulfonyl) propanoyl chloride (S6)

A 500 mL three neck round bottom flask equipped with a nitrogen inlet, reflux condenser, vent tube to 1 N NaOH base scrubber and stir bar was charged with 3-(methylsulfonyl) propanoic acid (50 g, 329 mmol) and toluene (299 ml) to give a heterogeneous solution. To this was added thionyl chloride (1.5 equiv) and the solution was heated to an internal temperature of 70-75° C. The reaction was stirred at this temperature with monitoring by NMR. The reaction was cooled to room temperature at which time significant solid formation was observed. To the slurry was added 250 mL heptane and the mixture was stirred for 10 minutes. The solids were isolated by filtration, washing with heptane (90% yield). ¹H NMR (500 MHz, CDCl₃) δ 3.51-3.46 (m, 2H), 3.43-3.38 (m, 2H), 3.00 (s, 1H); 13C NMR (126 MHz, CDCl3) δ 171.8, 49.5, 41.6, 39.3.

### Step 6: Preparation of N-[4-chloro-2-(pyridin-3-yl)-1,3-thiazol-5-yl]-N-ethyl-3-(methylsulfonyl) propanamide (Compound 1) as Form A

To a 1 L round bottomed flask containing crude 4-chloro-N-ethyl-2-(pyridin-3-yl)-1,3-thiazol-5-amine (27 g) was added acetonitrile (300 mL) making a red solution. 3-(Methylsulfonyl) propanoyl chloride (27.6 g, 162 mmol, 1.5 equiv.) was added in portions over 5 minutes and the reaction was monitored by HPLC until >98% conversion was achieved. During the course of the reaction the HCl salt of the product formed as a yellow/tan solid. The solid was isolated by filtration then neutralized with 20% potassium carbonate, extracting with ethyl acetate. The organic layer was dried over sodium sulfate, filtered, and concentrated giving 52.3 g of a crude oil. The crude material was crystallized from isopropanol-heptane to provide Compound 1 (35.2 g, 80%) as a crystalline solid. 1H NMR (300 MHz, Chloroform-d) δ 9.17-9.06 (m, 1H), 8.74 (dd, J=4.9, 1.6 Hz, 1H), 8.22 (ddd, J=8.0, 2.4, 1.6 Hz, 1H), 7.45 (ddd, J=8.0, 4.9, 0.9 Hz, 1H), 3.79 (q, J=7.2 Hz, 2H), 3.43 (s, 2H), 2.96 (s, 3H), 2.80 (t, J=7.1 Hz, 2H), 1.34-1.15 (m, 3H); ¹³C NMR (75 MHz, CDCl3) δ 169.43, 163.02, 152.15, 147.29, 138.62, 133.37, 131.86, 128.41, 123.92, 50.21, 45.26, 41.75, 27.29, 12.82; HRMS-ESI (m/z) [M+H]+ calcd for C₁₄H₁₆ClN₃O₃S₂, 373.0322; found, 374.0397. The crystalline solid was analyzed by PXRD according to Example 8, as shown in FIG. 1, and assigned the designation Form A.

### Example 2

### Synthesis of N-[4-chloro-2-(pyridin-3-yl)-1,3-thiazol-5-yl]-N-ethyl-3-(methylsulfonyl) propanamide (Compound 1), and Recrystallization as Form A

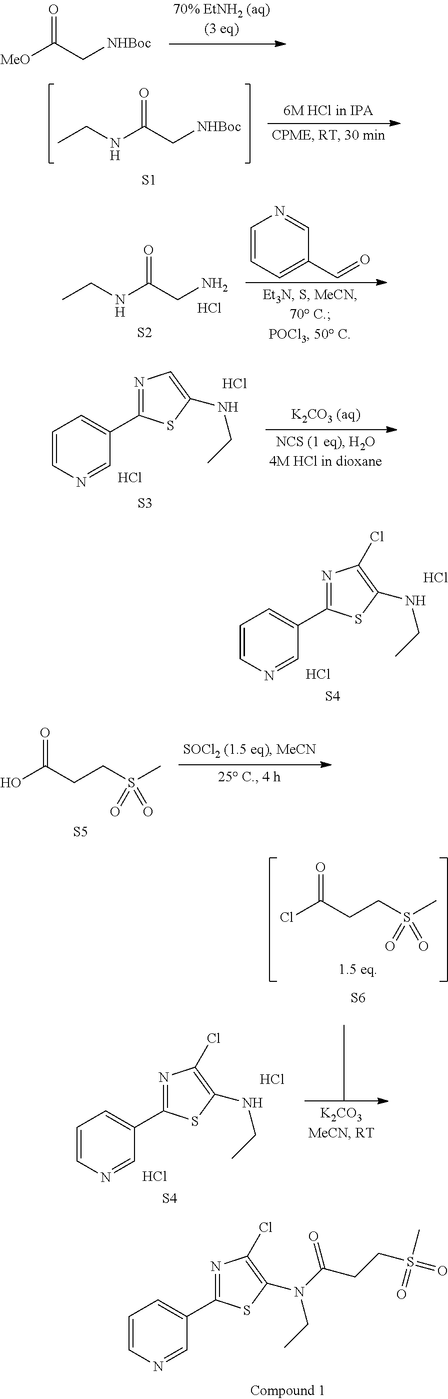

### Step 1: Preparation of tert-butyl (2-(ethylamino)-2-oxoethyl) carbamate (S1)

To a 3 L jacketed reactor fitted with a temperature logger, mechanical stirrer, low flow/high flow nitrogen setup was added ethylamine solution in water (70%) (599 mL, 7.53 mol). The reactor bath was set to 23° C. (internal temperature=22° C.). To this stirred solution was slowly added methyl (tert-butoxycarbonyl) glycinate (500 g, 2510 mmol) using a peristaltic pump keeping the internal temperature below 35° C. The reaction was stirred at 23° C. (internal temperature) overnight. In-process control (¹H NMR) showed complete conversion of the starting material to the product, tert-butyl (2-(ethylamino)-2-oxoethyl) carbamate (S1). The reactor was setup with a short path distillation head and the reactor bath was heated to 127° C. (internal temperature of ˜115° C., distillation head temperature fluctuated between 70-85° C.). The receiver flask was cooled in an ice bath and ˜240 mL of ethyl amine/water/MeOH was removed. The reaction was cooled to an internal temperature 60° C., and ˜350 mL of CPME was added, followed by a second distillation to remove the remaining ethylamine (bath temperature set to 117° C., internal temperature was ˜98° C., and the distillation head temperature was ˜80° C.). Upon distillation completion (determined by decrease in head temperature indicating a lack of distillate), the reactor bath was set to 23° C. (internal temperature=22° C.) and allowed to stir overnight. The product, tert-butyl (2-(ethylamino)-2-oxoethyl) carbamate, was used in the next step without further purification.

### Step 2: Preparation of 2-amino-N-ethylacetamide hydrochloride (S2)

To a 5 L jacketed reactor equipped with a nitrogen inlet, peristaltic pump, mechanical stirrer and an exhaust vent going through a base scrubber (alligator trap containing 10% NaOH solution) was added HCl (6 M in isopropyl alcohol) (1.67 L, 10.0 mol). The amide from Step 1 was added to the acid dropwise from the 3 L jacketed reactor to the 5 L jacketed reactor using the peristaltic pump. After the addition was complete, 400 mL of CPME was added into the 3 L reactor to wash out the reactor, and the CPME liquors were added to the 5 L reactor by peristaltic pump. The reaction was agitated overnight, during which time a white precipitate formed. The reactor contents were filtered to provide ˜100 g of 2-amino-N-ethylacetamide hydrochloride (S2), which was set aside for drying. The filtrate was concentrated to 1.3 L (˜50% volume), followed by addition of 600 mL of CPME and 100 mL of isopropyl alcohol to induce further precipitation of S2. The mixture was agitated for 60 minutes. The resultant slurry was filtered, and the solids were combined with the first crop of solids and dried in a vacuum oven overnight (50° C., <50 mmHg) to provide 2-amino-N-ethylacetamide hydrochloride as a white solid (300.5 g, 2168 mmol, 86% yield): ¹H NMR (400 MHz, DMSO-d₆) δ 3.49 (s, 2H), 3.14 (qd, J=7.2, 5.4 Hz, 2H), 1.05 (t, J=7.2 Hz, 3H). ESIMS m/z 103 [(M+H)⁺].

### Step 3: Preparation of N-ethyl-2-(pyridin-3-yl)-1,3-thiazol-5-amine dihydrochloride (S3)

To a 5 L jacketed reactor equipped with 2-reflux condensers (dry ice), mechanical stirrer, N₂ inlet, and thermocouple was added 2-amino-N-ethylacetamide hydrochloride (S2) (238 g, 1.72 mol) and anhydrous acetonitrile (2.00 L) leading to a white slurry. The reactor was inerted with nitrogen. Anhydrous triethylamine (264 ml, 1.89 mmol) was added, and the mixture was stirred for 1 h leading to a white thick slurry. Nicotinaldehyde (131 ml, 1.37 mol) was added leading to a thinner slurry. Then, sulfur (57.2 g, 1.78 mol) was added. The jacketed temperature was set to 72° C. (internal temperature=70° C.) and gradually (within 30 min) became a dark red orange solution and precipitates formed. The reaction was monitored by HPLC for disappearance of nicotinaldehyde which took ˜5 h of heating at 72° C. and then cooled to room temperature and stirred overnight. The heterogeneous reaction was reheated to an internal temperature 50° C. (where it once again became a dark red/brown clear solution). To the reaction at an internal temperature of 50° C. was added phosphoryl trichloride (321 ml, 3431 mmol) dropwise to not increase the internal temperature past 65.5° C. The reaction was monitored by HPLC until the thioamide intermediate was consumed (took 6 hours). The jacketed temperature was lowered to 20° C. (internal temperature=20° C.), and the solids were isolated by filtration and washed with acetonitrile. The reactor was washed with 100 mL acetonitrile. The filtrates were removed to be quenched. The solid was further washed with 300 mL DCM. The yellow/green wet cake was dried under vacuum at (40° C., <50 mmHg) for 16 h to afford N-ethyl-2-(pyridin-3-yl)-1,3-thiazol-5-amine dihydrochloride as green solid (200 g, 703 mmol, 51% yield): ¹H NMR (400 MHz, CDCl₃) δ 8.98 (dd, J=2.3, 0.9 Hz, 1H), 8.53 (dd, J=4.8, 1.6 Hz, 1H), 8.07 (ddd, J=8.0, 2.3, 1.6 Hz, 1H), 7.31 (ddd, J=8.0, 4.8, 0.9 Hz, 1H), 6.98 (s, 1H), 3.95 (s, 1H), 3.24 (q, J=7.2 Hz, 2H), 1.31 (t, J=7.2 Hz, 3H). ESIMS m/z 206 [(M+H)⁺].

### Step 4: Preparation of 4-chloro-N-ethyl-2-(pyridin-3-yl) thiazol-5-amine dihydrochloride (S4)

To a 5 L reactor equipped with a mechanical stirrer, N2 inlet and temperature probe was added N-ethyl-2-(pyridin-3-yl)-1,3-thiazol-5-amine dihydrochloride (210 g, 755 mmol) and water (630 mL) followed by ethyl acetate (2.10 L). To this stirred dark red solution was added a solution of potassium carbonate (209 g, 1.51 mol) in water (209 mL) dropwise via peristaltic pump over ten minutes. Upon completion, the jacket temperature was set to 45° C. (internal temperature=44° C.) and the reaction was stirred for 2 h. The reaction was then transferred to a separatory funnel and the bottom aqueous layer was removed. The aqueous layer was a light clear orange color. The aqueous layer was discarded (HPLC showed no desired product). The organic layer was poured into an Erlenmeyer flask equipped with a magnetic stirrer and MgSO₄(210 g). The flask was stirred for 3 h until the water level of the organic solution measured <0.5 wt % by Karl Fischer titration. The mixture was filtered, and the inorganic solids were washed with EtOAc (100 mL). The filtrate was poured back into the 5 L reactor and cooled to an internal temperature of 0° C., during which time the dark orange solution turned heterogeneous. To the heterogeneous slurry was added 1-chloropyrrolidine-2,5-dione (101 g, 755 mmol) as a solid, maintaining the internal temperature below 12° C. After the addition was complete, the reaction became homogeneous, and the reaction was monitored by HPLC. Ten minutes after the addition was complete, the reaction was determined to be complete by HPLC. 4 M HCl in dioxane (566 mL, 2.27 mol) was then dropwise via a peristaltic pump over 90 minutes. The jacketed temperature was set to 23° C. (internal temperature=22° C.) and the reaction was allowed to stir at room temperature overnight. After stirring overnight, the heterogeneous slurry was filtered to give a yellow/brown solid that was dried in a vacuum oven (30° C., <50 mmHg) to obtain 4-chloro-N-ethyl-2-(pyridin-3-yl) thiazol-5-amine dihydrochloride (S4) as a yellow/brown solid (195 g, 629 mmol, 83% yield): ¹H NMR (400 MHz, DMSO-d₆) δ 9.05 (d, J=2.2 Hz, 1H), 8.66 (dd, J=5.3, 1.4 Hz, 1H), 8.45 (dt, J=8.4, 1.8 Hz, 1H), 7.78 (dd, J=8.2, 5.2 Hz, 1H), 3.19 (q, J=7.2 Hz, 2H), 1.23 (t, J=7.1 Hz, 3H). ESIMS m/z 240 [(M+H)⁺].

### Step 5: Preparation of 3-(methylsulfonyl) propanoyl chloride (S6)

3-(methylsulfonyl) propanoic acid (S5) (101 g, 663 mmol, purchased from Orchev) was charged into a 1 L jacketed reactor followed by acetonitrile (304 g) and then agitated to dissolve the acid. Thionyl chloride (83.7 g, 697 mmol) was added dropwise over five minutes. The solution was held at 25° C. for 3 hours to allow the acid (S5) to convert to the acid chloride, 3-(methylsulfonyl) propanoyl chloride (S6). The acid chloride product was used in the next step without further purification.

### Step 6: Preparation of N-[4-chloro-2-(pyridin-3-yl)-1,3-thiazol-5-yl]-N-ethyl-3-(methylsulfonyl) propanamide (Compound 1)

A separate 5 L reactor was charged with 4-chloro-N-ethyl-2-(pyridin-3-yl) thiazol-5-amine dihydrochloride (164 g, 84 wt % pure, 442 mmol, 1.0 equiv.) followed by MeCN (415 g, 22.9 equiv.) and potassium carbonate (156 g, 1.11 mol). The orange slurry was stirred prior to the transfer of the acid chloride solution from the 1 L reactor. The solution of 3-(methylsulfonyl) propanoyl chloride prepared in Step 5 was transferred over 15 minutes via a peristaltic pump and an exotherm of +3° C. was observed. The reaction was stirred overnight at room temperature.

After stirring overnight the slurry was cooled to an internal temperature of 8° C. before water (554 g) was transferred to the 5 L reactor via a peristaltic pump over 3 hours. After the solids had dissolved, the agitation was stopped to allow the biphasic mixture to separate. The lower aqueous phase was discarded and the organic phase was then concentrated on a rotary evaporator at 50° C. to provide N-[4-chloro-2-(pyridin-3-yl)-1,3-thiazol-5-yl]-N-ethyl-3-(methylsulfonyl) propanamide (Compound 1) as a crude brown oil.

### Step 7: Isolation of N-[4-chloro-2-(pyridin-3-yl)-1,3-thiazol-5-yl]-N-ethyl-3-(methylsulfonyl) propanamide (Compound 1)

The oil from Step 6 was dissolved in 1-butanol (497 g) and the solution was reloaded into a 1 L reactor. The reactor was warmed to 30° C. and seeded with N-[4-chloro-2-(pyridin-3-yl)-1,3-thiazol-5-yl]-N-ethyl-3-(methylsulfonyl) propanamide (Compound 1, Form A) (8.18 g, 21.9 mmol). The slurry was held at 30° C. for 8 hours before cooling to 18° C. over 12 hours. The slurry was held at 18° C. for 4 hours and then subsequently cooled to 10° C. over 8 hours. The slurry was filtered and the wet cake was washed with heptane. The wet cake was dried in the oven at <50 mmHg at 50° C. In this instance an uncontrolled heating took place in the oven which resulted in some of the wet cake melting. The resultant solids (150 g, 90%) were used in the next step without further purification.

### Step 8: Recrystallization of N-[4-chloro-2-(pyridin-3-yl)-1,3-thiazol-5-yl]-N-ethyl-3-(methylsulfonyl) propanamide (Compound 1) as Form A

N-[4-chloro-2-(pyridin-3-yl)-1,3-thiazol-5-yl]-N-ethyl-3-(methylsulfonyl) propanamide (150 g, 401 mmol) from Step 7 was loaded into a 1 L reactor followed by MeOH (176 g). The reactor was padded with nitrogen and the agitation started. The contents were heated to 55° C. to dissolve the solid into solution. The dark brown solution was cooled to 25° C. and seeded with N-[4-chloro-2-(pyridin-3-yl)-1,3-thiazol-5-yl]-N-ethyl-3-(methylsulfonyl) propanamide (Compound 1, Form A) (1.50 g, 4.01 mmol). The slurry was held for 5 hours before water (253 g) was added to the reactor via a peristaltic pump over 3 hours. The slurry was held at 25° C. overnight. The next day the slurry was cooled to an internal temperature of 4° C. over 4 hours before it was filtered. The wet cake was washed with 10% MeOH in water (100 g) and then dried in a vacuum oven at 55° C. to give Compound 1 (92.15 g, 61%, 99.3 wt % purity) as a crystalline solid. The solid was analyzed by PXRD according to Example 8, as shown in FIG. 2, and assigned the designation Form A. ¹H NMR (400 MHz, CDCl₃) δ 9.12 (d, J=2.3 Hz, 1H), 8.77-8.71 (m, 1H), 8.22 (dt, J=8.1, 2.0 Hz, 1H), 7.45 (dd, J=8.1, 4.8 Hz, 1H), 3.79 (q, J=7.2 Hz, 2H), 3.43 (s, 2H), 2.96 (s, 3H), 2.80 (t, J=7.1 Hz, 2H), 1.23 (t, J=7.2 Hz, 3H). ESIMS m/z 374 [(M+H)⁺].

### Example 3

### Crystallization of N-[4-chloro-2-(pyridin-3-yl)-1,3-thiazol-5-yl]-N-ethyl-3-(methylsulfonyl) propanamide (Compound 1) as Form A from 1,4-dioxane

Dissolved 25.2 mg of Compound 1 Form A, as prepared in Example 2, in 500 μL of 1,4-dioxane in a 1 dram vial. The vial was covered with aluminum foil having pinhole in the foil, and placed vial in fume hood to evaporate solvent for 3 days to provide a crystalline solid. A sample was analyzed by PXRD according to Example 9, and assigned the designation Form A.

### Example 4

### Alternate crystallization of N-[4-chloro-2-(pyridin-3-yl)-1,3-thiazol-5-yl]-N-ethyl-3-(methylsulfonyl) propanamide (Compound 1) as Form A from MeOH

Dissolved 24.9 mg of Compound 1 Form A, as prepared in Example 2, in 1 mL MeOH in a 1 dram vial. The vial was covered with aluminum foil having pinhole in the foil, and placed in fume hood to evaporate the solvent for 3 days to provide a crystalline solid. A sample was analyzed by PXRD according to Example 9, and assigned the designation Form A.

### Example 5

### Crystallization of N-[4-chloro-2-(pyridin-3-yl)-1,3-thiazol-5-yl]-N-ethyl-3-(methylsulfonyl) propanamide (Compound 1) as Form B from acetonitrile

Dissolved 24.6 mg of Compound 1 Form A, as prepared in Example 2, in 500 μL acetonitrile in a 1 dram vial. The vial was covered with aluminum foil having pinhole in the foil, and placed in fume hood to evaporate the solvent for 3 days to provide a crystalline solid. A sample was analyzed by PXRD according to Example 9, as shown in FIG. 3, and assigned the designation Form B.

### Example 6

### Alternate crystallization of N-[4-chloro-2-(pyridin-3-yl)-1,3-thiazol-5-yl]-N-ethyl-3-(methylsulfonyl) propanamide (Compound 1) as Form B from EtOH

Dissolved 25.2 mg of Compound 1 Form A, as prepared in Example 2, in 2 mL EtOH in a 1 dram vial. The vial was covered with aluminum foil having pinhole in the foil, and placed in fume hood to evaporate the solvent for 3 days to provide a crystalline solid. A sample was analyzed by PXRD according to Example 9, and assigned the designation Form B.

### Example 7

### Evaporative crystallization solvent screen of N-[4-chloro-2-(pyridin-3-yl)-1,3-thiazol-5-yl]-N-ethyl-3-(methylsulfonyl) propanamide (Compound 1)

Following the same general procedure as described in Examples 3-6, Compound 1 Form A, as prepared in Example 2, was dissolved in various solvents and the evaporative crystallization procedure was carried out. The results are summarized in Table 4, and PXRD patterns were confirmed according to Example 9.

TABLE 4

| | | Polymorph |
|---|---|---|
| Solvent | Condition | Form |
| Acetone | Foil covered vial, 1 | A |
| | pinhole, room temp | |
| Dichloromethane | Foil covered vial, 1 | A |
| | pinhole, room temp | |
| Ethyl acetate | Foil covered vial, 1 | B |
| | pinhole, room temp | |
| iso-Propyl alcohol | Foil covered vial, 1 | B |
| | pinhole, room temp | |
| iso-Propyl acetate | Foil covered vial, 1 | B |
| | pinhole, room temp | |
| 2-Methyl-tetrahydrofuran | Foil covered vial, 1 | B |
| | pinhole, room temp | |
| Tetrahydrofuran | Foil covered vial, 1 | B |
| | pinhole, room temp | |

### Example 8

### Powder X-ray Diffraction (PXRD) of crystalline polymorph Forms A and B of Compound 1

Samples were analyzed using a Rigaku Miniflex II Benchtop X-ray diffractomer. The X-ray source is a Cu Normal Focus tube operated at 30 kV and 15 mA. Additional operating parameters are provided in table below.

| | | Scan speed/ | | |
|---|---|---|---|---|
| X-Ray | 30 kV, 15 mA | Duration time | 0.2000 deg./min. | |
| Goniometer | | Step width | 0.0100 | deg. |
| Attachment | — | Scan axis | 2theta/theta | |
| Filter | | Scan range | 3.0000-40.0000 | deg. |
| CBO selection slit | — | Incident slit | 1.25 | deg. |
| Diffracted beam mono. | Fixed Monochromator | Length limiting slit | — | |
| Detector | MiniFlex2 counter | Receiving slit #1 | 1.25 | deg. |
| Scan mode | CONTINUOUS | Receiving slit #2 | 0.3 | mm |

Powder samples were prepared by adding at least 20 mg to a glass sample holder and using light manual pressure to keep the sample surfaces flat and level with the reference surface of the sample holder. The glass holder was placed on top of an aluminum support. Each sample was analyzed from 3 to 40° 2θ using a continuous scan of 5° 2θ per minute with an effective step size of 0.02° 2θ. High resolution samples were analyzed using a continuous scan of 0.2° 2θ per minute and an effective step size of 0.01° 2θ.

### Example 9

### Alternative Powder X-ray Diffraction (PXRD) of crystalline polymorph Forms A and B of Compound 1

Samples were analyzed using a Rigaku Smart-Lab X-ray diffraction system configured for reflection BraggBrentano geometry using a line source X-ray beam. The x-ray source was a Cu Long Fine Focus tube that was operated at 40 kV and 44 ma. That source provided an incident beam profile at the sample that changed from a narrow line at high angles to a broad rectangle at low angles. Beam conditioning slits were used on the line X-ray source to ensure that the maximum beam size was less than 10 mm both along the line and normal to the line. The Bragg-Brentano geometry was a para-focusing geometry controlled by passive divergence and receiving slits with the sample itself acted as the focusing component for the optics. The inherent resolution of Bragg-Brentano geometry was governed in part by the diffractometer radius and the width of the receiving slit used. Typically, the Rigaku Smart-Lab was operated to give peak widths of 0.1° 2θ or less. The axial divergence of the X-ray beam was controlled by 5.0-degree Soller slits in both the incident and diffracted beam paths. Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

### Example 10

### Differential Scanning Calorimetry (DSC) of Polymorph Forms A and B of Compound 1

DSC analyses were carried out using a TA Instruments Q2500 DSC. Indium calibration and verification were completed prior to running the samples. A ˜3-5 mg sample of polymorph form A or B was loaded into a hermetically sealed aluminum pan under air. The sample was heated at a rate of 10° C./minute from 0° C. to 120° C. in the TA Q2500 DSC. The resulting thermograms were analyzed using the TRIOS software to calculate melting point and heat of fusion. Results are shown in FIG. 4 (Polymorph Form A) and FIG. 5 (Polymorph Form B).

### Example 11

### Low Frequency-Raman Spectrum of Polymorph Form A and B of Compound 1

Raman spectra were acquired using a Kaiser RXN2 spectrometer equipped with a 785 nm Invictus laser (15 mW). The spectra were collected using a collimated-beam probe with Kaiser's cosmic ray suppression and acquisition times of 4 minutes per spectrum. Results are shown in FIG. 6 (Polymorph Form A) and FIG. 7 (Polymorph Form B).

### Example 12

### Air Milling of Polymorph A and Polymorph B Materials

Compound 1, Polymorph A and Compound 1, Polymorph B samples were air milled separately with Jet Mill until particle size of d (0.5)<6 μm and d (0.9)<15 μm. Powder X-Ray Diffraction (PXRD) was conducted on the samples before and after the air milling process. The PXRD data demonstrates that the polymorphic form of the sample was unchanged by the milling process. After milling, the PXRD data showed that the milled Compound 1, Polymorph A was consistent with the PXRD data for Compound 1, Polymorph A described herein. After milling, the PXRD data showed that the milled Compound 1, Polymorph B was consistent with the PXRD data for Compound 1, Polymorph B described herein.

### Example 13

Comparison of Crystalline Compound 1, Polymorph A and Compound 1, Polymorph B to Non-Crystalline Compound 1 (an Amorphous Oil) in Head-to-Head Adult Sweet Potato Whitefly, B. tabaci, Knock-Down Studies

In this study, the effect of Compound 1 on the behavior of adult sweet potato whitefly, B. tabaci was evaluated. In general, different forms of Compound 1 (either inventive Polymorph form A, inventive Polymorph form B, or non-crystalline Compound 1, amorphous oil) were applied to the inside of glass vials and adult B. tabaci were transferred into treated vials, exposing them to Compound 1 via contact with the treated glass surface. To quantify the knock-down effect reported previously (Lee et. al. 2013), a line was scribed 2 cm above the bottom of the treated vials and the number of B. tabaci above the 2 cm line were recorded the first three hours following initial exposure. While the whiteflies that were knocked-down were not immediately killed, in field-simulated tests and field trials on treated plants, this effect has been shown to lead to mortality through starvation and desiccation.

This study characterized the effect of Compound 1, Polymorph A and Compound 1, Polymorph B on adult B. tabaci whitefly behavior using a common treated glass vial bioassay to expose the test insects through direct contact (Busvine 1971), and compared those effects in direct head-to-head comparison with non-cystalline Compound 1. Test compounds were either dissolved in acetone (Example 11A) or suspended in a volatile anti-solvent (hexane) (Example 11B), and then evenly coated on the inside of a glass vial using a laboratory-roller.

### Example 13A

### Acetone Experiment

In this experiment, two forms of Compound 1 (polymorph form B and an amorphous oil) were evaluated for their effect on B. tabaci behavior. Three application rates were evaluated for each form of Compound 1 (25 g/ha, 2.5 g/ha,and 0.25 g/ha), in addition to a solvent blank control. Each treatment and the control were replicated three times. Compound 1 samples were dissolved in acetone and were coated on the inside of a 11-dram screw-top glass vial (Fisherbrand 03-339-21N) (Fisher Scientific, Hampton, NH). Each vial had an internal dimension of 2.5×9 cm resulting in a treatment area of 75.6 cm². To treat the vials, a stock solution of each sample was first prepared by dissolving 1 mg of Compound 1 sample (either polymorph form B or an amorphous oil) in 2 ml of acetone. The stock solution was agitated using a laboratory vortex mixer to ensure complete mixture of the solution. 151 μL of each stock solution was added to 11.85 mL of acetone to create the treatment solution for the 25 g/ha rate (resulting in 0.25 μg/cm²). The 25 g/ha treatment solution was serially diluted ten-fold, two times to generate treatment solutions for the 2.5 and 0.25 g/ha rates. 3 mL of each treatment solution was transferred into the 11-dram vials and the vials were placed on a laboratory tube roller and rotated at room temperature until the acetone had fully evaporated, leaving a coating of Compound 1 on the inside of the vials. The solvent blank vials were treated with 3 mL of acetone. After treatment, the vials were left open in a fume hood overnight at room temperature in preparation for testing the following day. Mixed-sex adult B. tabaci whiteflies (Middle East Asia Minor1 (MEAM1) biotype) were collected from a susceptible colony maintained at Corteva Agriscience (Indianapolis, IN). The adult B. tabaci were anesthetized with CO₂ and approximately 66.5, 32-102 (Mean, min-max) were introduced into each of the treated vials and their behavior recorded. To evaluate B. tabaci behavior, a line was scribed 2 cm above the bottom of the vial and the number of individuals above the line were recorded at 15, 30, 45, 60, 90, 120, 150, and 180 min after introduction. Following completion of the experiment, the insects were devitalized by freezing at −30° C. for 72 h after which the total number of insects in each vial was recorded. The percentage of B. tabaci above the 2 cm line was calculated for each rate and was used to graph the effect of Compound 1 on whiteflies over time.

### Statistical Analysis

For each trial and rate, the proportion of insects above the 2 cm line was analyzed with a generalized linear mixed model (GLMM) for repeated measures with binomial response and logit link function (Stroup, 2012). The use of the generalized linear model with binomial distribution for the response, instead of the linear model with normal distribution (ANOVA), allows the correct use of the distributional assumptions and actual sample size (number of insects) used in the experiments.

The model included Treatment, Time point, and the interaction Treatment x Time point as fixed effects and the replicate (experimental unit) as random effect. The correlation between repeated measures was modeled with the compound symmetry covariance matrix.

The generalized linear mixed models were estimated with the residual pseudo-likelihood method, and the means of the treatment proportions were compared with Tukey's tests (α=0.05) (Stroup 2012). Statistical analyses were performed with SAS Proc GLIMMIX (SAS Software, Version 9.4, SAS Institute Inc., Cary, NC).

### Results

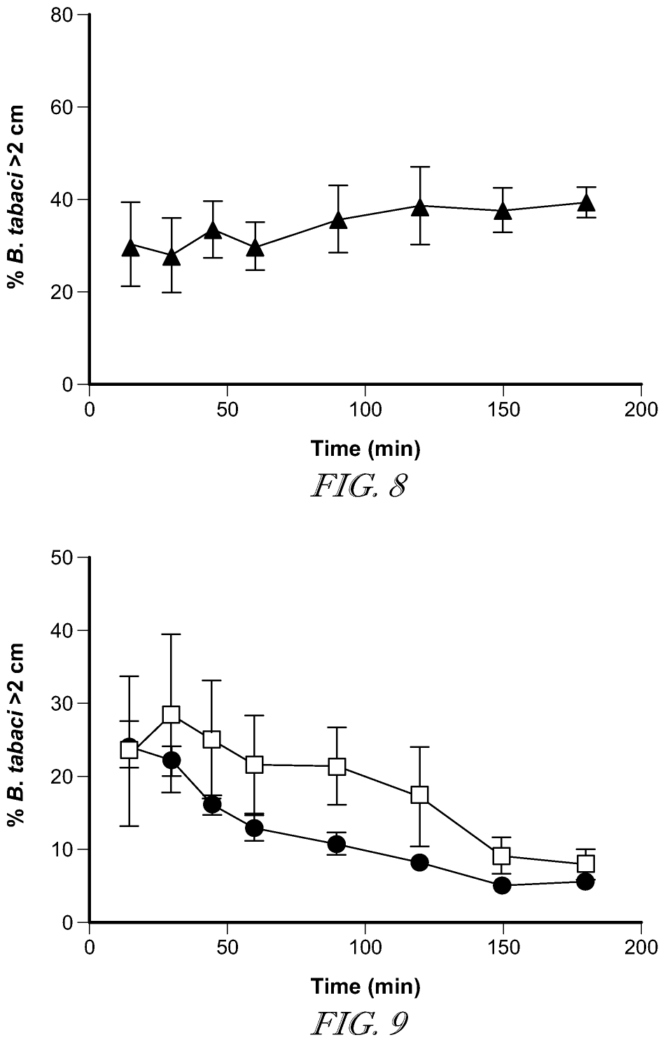

In the solvent blank controls, approximately 20-30% of insects were above the 2 cm line at any given time (34.1%±2.18, Mean±SEM) (FIG. 8). At the highest rate of 25 g/ha, there was a significant effect of Compound 1 exposure over time, evidenced by the reduction in the number of insects above the 2 cm line over time as exposure to Compound 1 began to negatively affect their behavior (FIG. 9, Table 5). However, there was no significant effect of treatment and no interaction between treatment and time point, indicating that there was no difference in the effect of polymorph B and the amorphous oil at the 25 g/ha rate (Table 5). At the middle rate of 2.5 g/ha, there was a significant difference between the effect of polymorph B and the amorphous oil, where fewer insects exposed to polymorph B were above the 2 cm line because their behavior was more affected than insects exposed to the amorphous oil at this rate (FIG. 10, Table 6). At the 2.5 g/ha rate, there was no effect of time or interaction between treatment and time (Table 6). Finally, at the lowest rate of 0.25 g/ha, there was no significant effect of any of the model effects indicating that there was not a difference in the effect of polymorph B and the amorphous oil at this rate (Table 7).

TABLE 5
Type III Tests of Fixed Effects

| | Num | Den | F | |
|---|---|---|---|---|
| Effect | DF | DF | Value | Pr > F |
| Trt | 1 | 4.127 | 1.06 | 0.359 |
| Time point | 7 | 30.11 | 18.57 | <.0001 |
| Trt × Time point | 7 | 30.11 | 1.55 | 0.1883 |

Trt Least Squares Means (proportion scale)

| | | | Tukey-Kramer |
|---|---|---|---|
| | | | Grouping for Trt |
| | | Standard | Least Squares |
| | | Error | Means |
| Trt | Mean | mean | (Alpha = 0.05) |
| amorphous oil | 0.167 | 0.042 | a |
| Polymorph B | 0.115 | 0.030 | a |

TABLE 6
Type III Tests of Fixed Effects

| | Num | Den | F | |
|---|---|---|---|---|
| Effect | DF | DF | Value | Pr > F |
| Trt | 1 | 4.278 | 17.31 | 0.0123 |
| Time point | 7 | 26.5 | 0.65 | 0.7085 |
| Trt × Time point | 7 | 26.5 | 0.52 | 0.81 |

Trt Least Squares Means (proportion scale)

| | | | Tukey-Kramer |
|---|---|---|---|
| | | | Grouping for |
| | | Standard | Trt Least |
| | | Error | Squares Means |
| Trt | Mean | mean | (Alpha = 0.05) |
| amorphous oil | 0.326 | 0.035 | a |
| Polymorph B | 0.156 | 0.022 | b |

TABLE 7
Type III Tests of Fixed Effects

| | Num | Den | F | |
|---|---|---|---|---|
| Effect | DF | DF | Value | Pr > F |
| Trt | 1 | 3.976 | 4.36 | 0.1054 |
| Time point | 7 | 25.51 | 0.53 | 0.8014 |
| Trt × Time point | 7 | 25.51 | 0.28 | 0.9554 |

Trt Least Squares Means (proportion scale)

| | | | Tukey-Kramer |
|---|---|---|---|
| | | | Grouping for Trt |
| | | Standard | Least Squares |
| | | Error | Means |
| Trt | Mean | mean | (Alpha = 0.05) |
| amorphous oil | 0.295 | 0.062 | a |
| Polymorph B | 0.502 | 0.074 | a |

### Example 13B

### Hexane Experiment

In this experiment, three forms of Compound 1 (Polymorph form A, Polymorph form B, and non-crystalline, amorphous oil) were evaluated for their effect on B. tabaci behavior, polymorph A, polymorph B and an amorphous oil. The samples of Polymorph form A and Polymorph form B were finely milled as described in Example 12. The experimental design of the hexane experiment, including rates (25, 2.5, and 0.25 g/ha), replication (three replications per treatment), and preparation of the treatment suspension was similar to the process described for Example 13A. However, hexane was used instead of acetone. Because Compound 1 does not readily dissolve in hexane, it was used as an anti-solvent to suspend particles of each polymorph form of Compound 1 and the non-crystalline, amorphous oil Compound 1 for application to the inner surface of the glass vials, while retaining the polymorph structure. Because Compound 1 does not readily dissolve in hexane, to distribute the particles in each vial as consistently as possible, the stock suspensions were mixed using a laboratory vortex mixer for 30 seconds, followed by agitation for 5 minutes in an ultrasonic bath (Branson 2800, Branson Ultrasonics Corp., Danbury, CT). Following sonication, samples were again mixed using a vortex mixer immediately before transferring 151 μl of each stock suspension to 11.85 mL of hexane to create the treatment suspension for the 25 g/ha rate (resulting in 0.25 μg/cm²). The 25 g/ha treatment suspension was serially diluted ten-fold, two times to generate treatment suspensions for the 2.5 and 0.25 g/ha rates. 3 mL of each treatment suspension was transferred into the 11-dram vials and the vials were placed on a laboratory tube roller and rotated at room temperature until the hexane had fully evaporated, leaving an even coating of Compound 1 particles on the inside of the vials. The solvent blank vials were treated with 3 mL of hexane. Following treatment, the vials were stored, infested with approximately 117.4, 47-219 (Mean, min-max) adult B. tabaci, and evaluated as described in the acetone experiment.

### Statistical Analysis

For each trial and rate, the proportion of insects above the 2 cm line was analyzed with a generalized linear mixed model (GLMM) for repeated measures with binomial response and logit link function (Stroup, 2012). The use of the generalized linear model with binomial distribution for the response, instead of the linear model with normal distribution (ANOVA), allows the correct use of the distributional assumptions and actual sample size (number of insects) used in the experiments.

The model included Treatment, Time point, and the interaction Treatment x Time point as fixed effects and the replicate (experimental unit) as random effect. The correlation between repeated measures was modeled with the compound symmetry covariance matrix.

The generalized linear mixed models were estimated with the residual pseudo-likelihood method, and the means of the treatment proportions were compared with Tukey's tests (α=0.05) (Stroup 2012). Statistical analyses were performed with SAS Proc GLIMMIX (SAS Software, Version 9.4, SAS Institute Inc., Cary, NC).

### Results

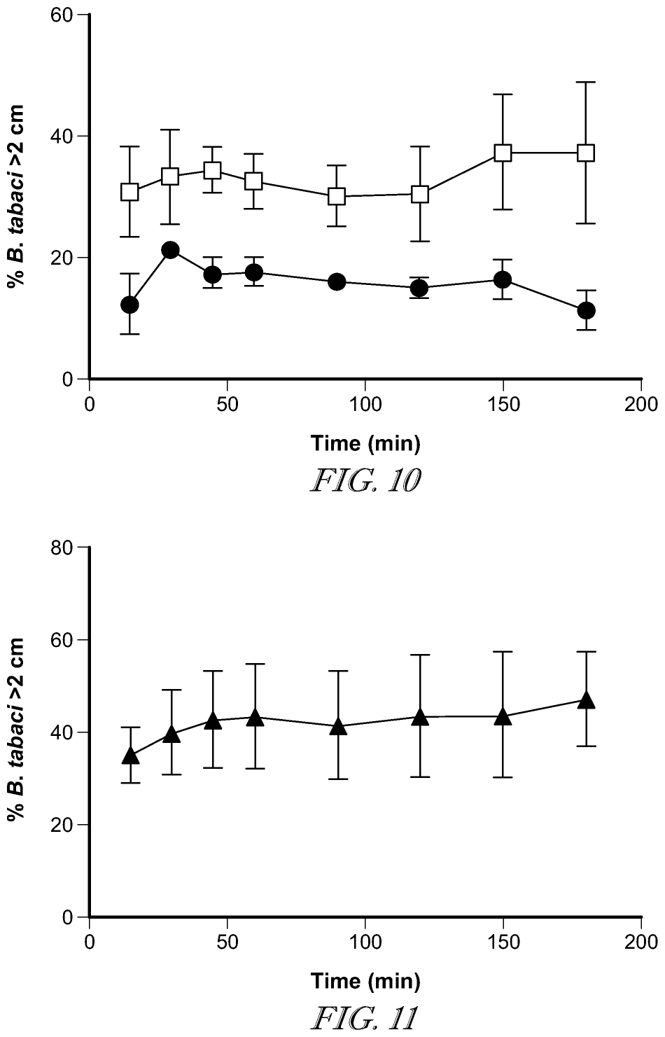

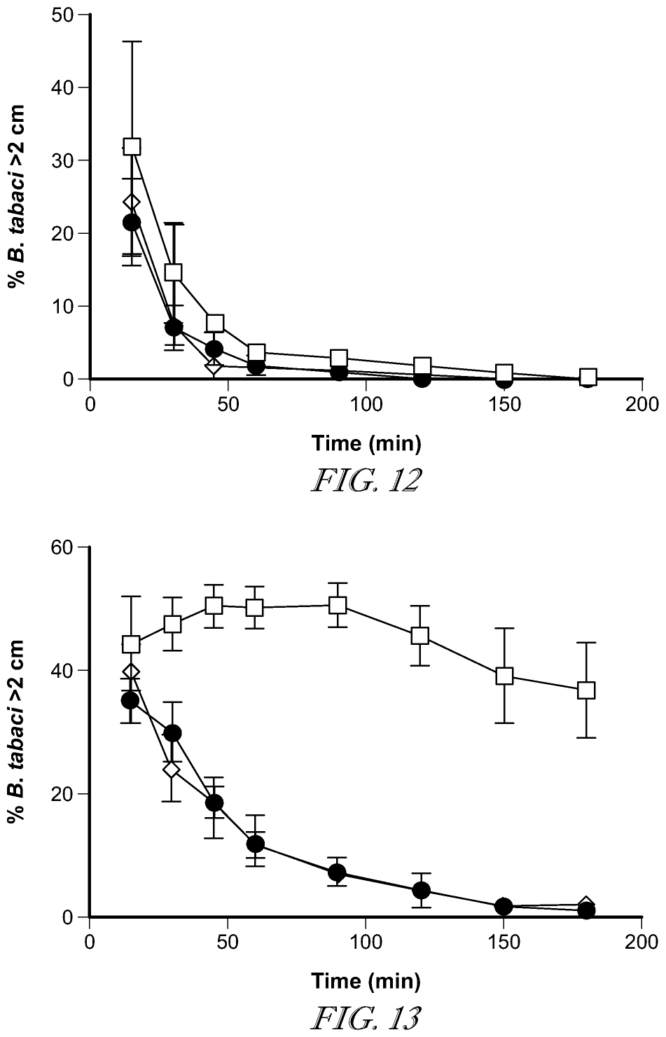

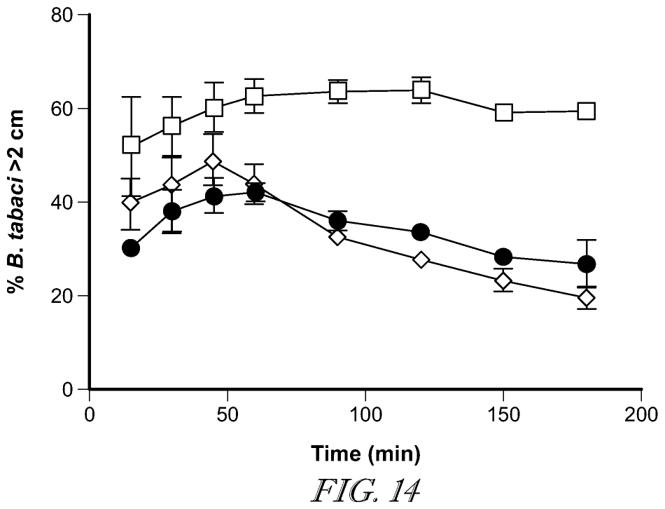

In the solvent blank controls, approximately 30-50% of insects were above the 2 cm line at any given time (42.1%±3.31, Mean±SEM) (FIG. 11). Similar to the acetone experiment, at the highest rate (25 g/ha) there was a significant effect of Compound 1 exposure over time for both the polymorphic forms and the amorphous oil, evidenced by the number of insects above the 2 cm line decreased over time as the insecticides began to negatively affect their behavior (FIG. 12, Table 8). However, there was no significant effect of treatment indicating that there was not a difference in the effect of polymorphs A, B and the amorphous oil at this rate (Table 8). At the middle rate of 2.5 g/ha, there was a significant effect of treatment, where whiteflies exposed to either polymorph form A or polymorph form B were significantly affected when compared directly to the amorphous oil (FIG. 13, Table 9). In addition, at the 2.5 g/ha rate there was a significant effect of Compound 1 exposure over time, which was very evident in both polymorph form A and polymorph form B treatments (FIG. 13, Table 9). Finally, there was a significant interaction of treatment and time at the 2.5 g/ha rate, indicating that there are differences in how insects in each treatment responds over time, as evidenced in the different shapes of the response curves between the polymorph form A and polymorph form B treatments compared to the amorphous oil over time (FIG. 13, Table 9). At the lowest rate (0.25 g/ha), results similar to those observed at the 2.5 g/ha rate were obtained. At 0.25 g/ha, there were significant effects of treatment, time, and an interaction of treatment and time (FIG. 14, Table 10). Like the 2.5 g/ha rate, insects exposed at the 0.25 g/ba rate with polymorph form A and polymorph form B treatments were more negatively affected than those exposed to the amorphous oil (FIG. 14, Table 10) at the 0.25 g/ha rate.

TABLE 8
Type III Tests of Fixed Effects

| | Num | Den | F | |
|---|---|---|---|---|
| Effect* | DF | DF | Value | Pr > F |
| Trt | 2 | 5.737 | 1.32 | 0.3366 |
| Time point | 7 | 62 | 40.81 | <.0001 |

Trt Least Squares Means (proportion scale)

| | | | Tukey-Kramer |
|---|---|---|---|
| | | | Grouping for Trt |
| | | Standard | Least Squares |
| | | Error | Means |
| Trt | Mean | mean | (Alpha = 0.05) |
| amorphous oil | 0.029 | 0.011 | a |
| Polymorph B | 0.013 | 0.005 | a |
| Polymorph A | 0.015 | 0.006 | a |

*Interaction Trt × Time point, non-significant effect, was removed from the model to reach convergence.

TABLE 9
Type III Tests of Fixed Effects

| | Num | Den | F | |
|---|---|---|---|---|
| Effect | DF | DF | Value | Pr > F |
| Trt | 2 | 6.859 | 35.45 | 0.0002 |
| Time point | 7 | 48 | 50.19 | <.0001 |
| Trt × Time point | 14 | 48 | 13.84 | <.0001 |

Trt Least Squares Means (proportion scale)

| | | | Tukey-Kramer |
|---|---|---|---|
| | | | Grouping for Trt |
| | | Standard | Least Squares |
| | | Error | Means |
| Trt | Mean | mean | (Alpha = 0.05) |
| amorphous oil | 0.453 | 0.053 | a |
| Polymorph B | 0.079 | 0.017 | b |
| Polymorph A | 0.080 | 0.017 | b |

TABLE 10
Type III Tests of Fixed Effects

| | Num | Den | F | |
|---|---|---|---|---|
| Effect | DF | DF | Value | Pr > F |
| Trt | 2 | 5.764 | 23.38 | 0.0017 |
| Time point | 7 | 40.95 | 10.47 | <.0001 |
| Trt × Time point | 14 | 40.42 | 4.1 | 0.0002 |

Trt Least Squares Means (proportion scale)

| | | | Tukey-Kramer |
|---|---|---|---|
| | | | Grouping for Trt |
| | | Standard | Least Squares |
| | | Error | Means |
| Trt | Mean | mean | (Alpha = 0.05) |
| amorphous oil | 0.601 | 0.030 | a |
| Polymorph B | 0.345 | 0.028 | b |
| Polymorph A | 0.342 | 0.028 | b |

1. A crystalline form of N-[4-chloro-2-(pyridin-3-yl)-1,3-thiazol-5-yl]-N-ethyl-3-(methylsulfonyl) propanamide.

2. The crystalline form of claim 1, wherein the crystalline form is a crystalline polymorph form of N-[4-chloro-2-(pyridin-3-yl)-1,3-thiazol-5-yl]-N-ethyl-3-(methylsulfonyl) propanamide.

3. The crystalline polymorph of claim 1, wherein the crystalline form is anhydrous or solvent-free.

4. A crystalline polymorph Form A of N-[4-chloro-2-(pyridin-3-yl)-1,3-thiazol-5-yl]-N-ethyl-3-(methylsulfonyl) propanamide having a powder X-ray diffraction pattern comprising a peak at diffraction angle (2θ) of 20.3±0.2.

5. The crystalline polymorph form of claim 4, wherein the crystalline polymorph form has a powder X-ray diffraction pattern comprising a peak at diffraction angle (2θ) of 17.4±0.2 and 20.3±0.2.

6. (canceled)

7. The crystalline polymorph form of claim 5, wherein the crystalline polymorph form has a powder X-ray diffraction pattern comprising peaks at diffraction angles (2θ) of 10.6±0.2, 17.4±0.2, 19.9±0.2, and 20.3±0.2.

8. (canceled)

9. The crystalline polymorph form of claim 7, wherein the crystalline polymorph form has a powder X-ray diffraction pattern comprising peaks at diffraction angles (2θ) of 10.6±0.2, 17.4±0.2, 18.2±0.2, 18.7±0.7, 19.9±0.2, and 20.3±0.2.

10. (canceled)

11. The crystalline polymorph form of claim 9, wherein the crystalline polymorph form has a powder X-ray diffraction pattern comprising peaks at diffraction angles (2θ) of 10.1±0.2, 10.6±0.2, 16.0±0.2, 17.4±0.2, 18.2±0.2, 18.7±0.7, 19.9±0.2, and 20.3±0.2.

12. (canceled)

13. The crystalline polymorph form of claim 11, wherein the crystalline polymorph form has a powder X-ray diffraction pattern comprising peaks at diffraction angles (2θ) of 10.0±0.2, 10.1±0.2, 10.6±0.2, 16.0±0.2, 17.4±0.2, 18.2±0.2, 18.7±0.7, 18.9±0.2, 19.9±0.2, and 20.3±0.2.

14. (canceled)

15. The crystalline polymorph form of claim 4, wherein the crystalline polymorph form has a DSC thermogram comprising an endothermal peak having a peak temperature at about 101.09° C.

16. (canceled)

17. The crystalline polymorph form of claim 4, having a Raman spectrum comprising one or more peaks at wavenumbers of about 255 cm⁻¹, about 441 cm⁻¹, about 539 cm⁻¹, about 778 cm⁻¹, about 921 cm⁻¹, about 991 cm⁻¹, about 1048 cm⁻¹, about 1123 cm⁻¹, about 1191 cm⁻¹, about 1526 cm⁻¹, about 1569 cm⁻¹ about 1588 cm⁻¹ about 1701 cm⁻¹, about 2949 cm⁻¹ and about 3053 cm⁻¹.

18. (canceled)

19. A crystalline polymorph Form B of N-[4-chloro-2-(pyridin-3-yl)-1,3-thiazol -5-yl]-N-ethyl-3-(methylsulfonyl) propanamide having a powder X-ray diffraction pattern comprising a peak at diffraction angle (2θ) of 15.4±0.2.

20. The crystalline polymorph form of claim 19, wherein the crystalline polymorph form has a powder X-ray diffraction pattern comprising peaks at diffraction angles (2θ) of 7.7±0.2 and 15.4±0.2.

21. (canceled)

22. The crystalline polymorph form of claim 20, wherein the crystalline polymorph form has a powder X-ray diffraction pattern comprising peaks at diffraction angles (2θ) of 7.7±0.2, 15.4±0.2, 16.7±0.2, and 20.2±0.2.

23.-25. (canceled)

26. The crystalline polymorph form of claim 24, wherein the crystalline polymorph form has a powder X-ray diffraction pattern comprising peaks at diffraction angles (2θ) of 7.7±0.2, 15.0±0.2, 15.4±0.2, 16.6±0.2, 16.7±0.2, 17.5±0.2, 18.4±0.2, and 20.2±0.2.

27. (canceled)

28. The crystalline polymorph form of claim 26, wherein the crystalline polymorph form has a powder X-ray diffraction pattern comprising peaks at diffraction angles (2θ) of 7.7±0.2, 15.0±0.2, 15.4±0.2, 16.6±0.2, 16.7±0.2, 17.3±0.2, 17.5±0.2, 18.4±0.2, 19.8±0.2, and 20.2±0.2.

29. (canceled)

30. The crystalline polymorph form of claim 19, wherein the crystalline polymorph form has a DSC thermogram comprising an endothermal peak having a peak temperature at about 105.24° C.

31. (canceled)

32. The crystalline polymorph form of claim 19, having a Raman spectrum comprising one or more peaks at wavenumbers of about 266 cm⁻¹, about 446 cm⁻¹, about 546 cm⁻¹, about 763 cm⁻¹, about 987 cm⁻¹, about 1044 cm⁻¹, about 1137 cm⁻¹, about 1187 cm⁻¹ and about 1308 cm⁻¹, about 1518 cm⁻¹, about 1573 cm⁻¹, about 1592 cm⁻¹, about 1673 cm⁻¹, about 2919 cm⁻¹, and about 2937 cm⁻¹.

33. (canceled)

34. A composition comprising the crystalline form according to claim 1.

35. A process to control a pest, said process comprising applying to a locus, a pesticidally effective amount of a crystalline form according to claim 1.
